SEQUENCE LISTING (91 sequences)

SEQ ID NO: 1 - protein, 8 aa, Mus musculus
DQRLYFDV

SEQ ID NO: 2 - protein, 16 aa, Mus musculus
VIWTDGGTNYNSPFMS

SEQ ID NO: 3 - protein, 5 aa, Mus musculus
TYDIS

SEQ ID NO: 4 - protein, 8 aa, Mus musculus
GQSFSYPT

SEQ ID NO: 5 - protein, 7 aa, Mus musculus
GASNRYT

SEQ ID NO: 6 - protein, 11 aa, Mus musculus
KASEDVNTYVS

SEQ ID NO: 7 - protein, 116 aa, Mus musculus
QVQLKESGPGLVAPSQSLSITCTVSGFSLTTYDISWIRQSPGKGLEWLGVIWTDGGTNYNSPFMSRLSIRKDNSKSQVFLKMNRLQTDDTAIYYCVRDQRLYFDVWGAGTTVTVSS

SEQ ID NO: 8 - protein, 106 aa, Mus musculus
NIVMTQSPKSMSMSVGERVTLNCKASEDVNTYVSWYQQQPEQSPKLLIYGASNRYTGVPDRFTGGGSTTDFTLTISSVQAEDLADYFCGQSFSYPTFGTGTKLEIK

SEQ ID NO: 9 - protein, 7 aa, Mus musculus
DPRLYFD

SEQ ID NO: 10 - protein, 16 aa, Mus musculus
VIWTGGGTNYNSGFMS

SEQ ID NO: 11 - protein, 5 aa, Mus musculus
SFDIS

SEQ ID NO: 12 - protein, 8 aa, Mus musculus
GQTFSYPT

SEQ ID NO: 13 - protein, 7 aa, Mus musculus
GASNRYT

SEQ ID NO: 14 - protein, 11 aa, Mus musculus
KASEDVVTYVS

SEQ ID NO: 15 - protein, 116 aa, Mus musculus
QVQLKESGPGLVAPSKSLSITCTVSGSSLDSFDISWIRQSPGKGLEWLGVIWTGGGTNYNSGFMSRLRITKDNSKSQVLLKMNSLQSDDTAIYYCVRDPRLYFDVWGAGTTVTVSS

SEQ ID NO: 16 - protein, 106 aa, Mus musculus
NIVMTQSPKSMSMSVGERVTLSCKASEDVVTYVSWYQQKPDQSPKLLIYGASNRYTGVPDRFTGSGSATDFTLTISSVQAEDLADYYCGQTFSYPTFGTGTKLEIK

SEQ ID NO: 17 - protein, 8 aa, Artificial
  heavy chain CDR3, hMab 2F11-c11
DQRLYFDV

SEQ ID NO: 18 - protein, 16 aa, Artificial
  heavy chain CDR2, hMab 2F11-c11
VIWTDGGTNYNSPFMS

SEQ ID NO: 19 - protein, 5 aa, Artificial
  heavy chain CDR1, hMab 2F11-c11
TYDIS

SEQ ID NO: 20 - protein, 8 aa, Artificial
  light chain CDR3, hMab 2F11-c11
GQSFSYPT

SEQ ID NO: 21 - protein, 7 aa, Artificial
  light chain CDR2, hMab 2F11-c11
GASNRYT

SEQ ID NO: 22 - protein, 11 aa, Artificial
  light chain CDR1, hMab 2F11-c11
RASEDVNTYVS

SEQ ID NO: 23 - protein, 116 aa, Artificial
  heavy chain variable domain, hMab 2F11-c11
QVQLVQSGAEVKKPGSSVKVSCKASGFSLTTYDISWIRQAPGQGLEWMGVIWTDGGTNYNSPFMSRVTITKDESTSTAYMELSSLRSEDTAVYYCVRDQRLYFDVWGQGTTVTVSS

SEQ ID NO: 24 - protein, 106 aa, Artificial
  light chain variable domain, hMab 2F11-c11
DIQMTQSPSSLSASVGDRVTITCRASEDVNTYVSWYQQKPGKAPKLLIYGASNRYTGVPSRFSGSGSGTDFTLTISSLQPEDFATYYCGQSFSYPTFGQGTKLEIK

SEQ ID NO: 25 - protein, 8 aa, Artificial
  heavy chain CDR3, hMab 2F11-d8
DQRLYFDV

SEQ ID NO: 26 - protein, 16 aa, Artificial
  heavy chain CDR2, hMab 2F11-d8
VIWTDGGANYAQKFQG

SEQ ID NO: 27 - protein, 5 aa, Artificial
  heavy chain CDR1, hMab 2F11-d8
TYDIS

SEQ ID NO: 28 - protein, 8 aa, Artificial
  light chain CDR3, hMab 2F11-d8
GQSFSYPT

SEQ ID NO: 29 - protein, 7 aa, Artificial
  light chain CDR2, hMab 2F11-d8
GASNRYT

SEQ ID NO: 30 - protein, 11 aa, Artificial
  light chain CDR1, hMab 2F11-d8
KASEDVNTYVS

SEQ ID NO: 31 - protein, 116 aa, Artificial
  heavy chain variable domain, hMab 2F11-d8
QVQLVQSGAEVKKPGSSVKVSCKASGFSLTTYDISWVRQAPGQGLEWMGVIWTDGGANYAQKFQGRVTITADESTSTAYMELSSLRSEDTAVYYCARDQRLYFDVWGQGTTVTVSS

SEQ ID NO: 32 - protein, 106 aa, Artificial
  light chain variable domain, hMab 2F11-d8
DIQMTQSPSSLSASVGDRVTITCKASEDVNTYVSWYQQKPGKAPKLLIYGASNRYTGVPSRFSGSGSGTDFTLTISSLQPEDFATYYCGQSFSYPTFGQGTKLEIK

SEQ ID NO: 33 - protein, 8 aa, Artificial
  heavy chain CDR3, hMab 2F11-e7
DQRLYFDV

SEQ ID NO: 34 - protein, 16 aa, Artificial
  heavy chain CDR2, hMab 2F11-e7
VIWTDGGTNYAQKLQG

SEQ ID NO: 35 - protein, 5 aa, Artificial
  heavy chain CDR1, hMab 2F11-e7
SYDIS

SEQ ID NO: 36 - protein, 8 aa, Artificial
  light chain CDR3, hMab 2F11-e7
QQSFSYPT

SEQ ID NO: 37 - protein, 7 aa, Artificial
  light chain CDR2, hMab 2F11-e7
AASNRYT

SEQ ID NO: 38 - protein, 11 aa, Artificial
  light chain CDR1, hMab 2F11-e7
RASEDVNTYVS

SEQ ID NO: 39 - protein, 116 aa, Artificial
  heavy chain variable domain, hMab 2F11-e7
QVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSS

SEQ ID NO: 40 - protein, 106 aa, Artificial
  light chain variable domain, hMab 2F11-e7
DIQMTQSPSSLSASVGDRVTITCRASEDVNTYVSWYQQKPGKAPKLLIYAASNRYTGVPSRFSGSGSGTDFTLTISSLQPEDFATYYCQQSFSYPTFGQGTKLEIK

SEQ ID NO: 41 - protein, 8 aa, Artificial
  heavy chain CDR3, hMab 2F11-f12
DQRLYFDV

SEQ ID NO: 42 - protein, 16 aa, Artificial
  heavy chain CDR2, hMab 2F11-f12
VIWTDGGTNYNSPFMS

SEQ ID NO: 43 - protein, 5 aa, Artificial
  heavy chain CDR1, hMab 2F11-f12
TYDIS

SEQ ID NO: 44 - protein, 8 aa, Artificial
  light chain CDR3, hMab 2F11-f12
GQSFSYPT

SEQ ID NO: 45 - protein, 7 aa, Artificial
  light chain CDR2, hMab 2F11-f12
GASSLQS

SEQ ID NO: 46 - protein, 11 aa, Artificial
  light chain CDR1, hMab 2F11-f12
RASEDVNTYVS

SEQ ID NO: 47 - protein, 116 aa, Artificial
  heavy chain variable domain, hMab 2F11-f12
QVQLVQSGAEVKKPGSSVKVSCKASGFSLTTYDISWVRQAPGQGLEWMGVIWTDGGTNYNSPFMSRVTITKDESTSTAYMELSSLRSEDTAVYYCVRDQRLYFDVWGQGTTVTVSS

SEQ ID NO: 48 - protein, 106 aa, Artificial
  light chain variable domain, hMab 2F11-f12
DIQMTQSPSSLSASVGDRVTITCRASEDVNTYVSWYQQKPGKAPKLLIYGASSLQSGVPSRFSGSGSGTDFTLTISSLQPEDFATYYCGQSFSYPTFGQGTKLEIK

SEQ ID NO: 49 - protein, 8 aa, Artificial
  heavy chain CDR3, hMab 2F11-g1
DQRLYFDV

SEQ ID NO: 50 - protein, 16 aa, Artificial
  heavy chain CDR2, hMab 2F11-g1
VIWTDGGTNYNSPLKS

SEQ ID NO: 51 - protein, 5 aa, Artificial
  heavy chain CDR1, hMab 2F11-g1
TYDIS

SEQ ID NO: 52 - protein, 8 aa, Artificial
  light chain CDR3, hMab 2F11-g1
GQSFSYPT

SEQ ID NO: 53 - protein, 7 aa, Artificial
  light chain CDR2, hMab 2F11-g1
GASSRAT

SEQ ID NO: 54 - protein, 11 aa, Artificial
  light chain CDR1, hMab 2F11-g1
RASEDVNTYLA

SEQ ID NO: 55 - protein, 116 aa, Artificial
  heavy chain variable domain, hMab 2F11-g1
QVQLQESGPGLVKPSETLSLTCTVSGFSLTTYDISWIRQPPGKGLEWIGVIWTDGGTNYNSPLKSRVTISVDTSKNQFSLKLSSVTAADTAVYYCARDQRLYFDVWGQGTTVTVSS

SEQ ID NO: 56 - protein, 106 aa, Artificial
  light chain variable domain, hMab 2F11-g1
EIVLTQSPGTLSLSPGERATLSCRASEDVNTYLAWYQQKPGQAPRLLIYGASSRATGIPDRFSGSGSGTDFTLTISRLEPEDFAVYYCGQSFSYPTFGQGTKLEIK

SEQ ID NO: 57 - protein, 107 aa, Homo sapiens [sequence omitted]

SEQ ID NO: 58 - protein, 330 aa, Homo sapiens [sequence omitted]

SEQ ID NO: 59 - protein, 330 aa, Artificial
  human heavy chain constant region derived from IgG1 mutated on L234A and L235A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 61 - protein, 327 aa, Artificial
  human heavy chain constant region derived from IgG4 mutated onS228P
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 62 - protein, 972 aa, Homo sapiens
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 63 - protein, 972 aa, Artificial
  mutant CSF-1R L301S Y969F
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYSNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNFQFC

SEQ ID NO: 64 - protein, 493 aa, Artificial
  human CSF-1R Extracellular Domain
IPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDE

SEQ ID NO: 65 - protein, 388 aa, Artificial
  human CSF-1R fragment delD4
IPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDE

SEQ ID NO: 66 - protein, 292 aa, Artificial
  human CSF-1R fragment D1-D3
IPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQ

SEQ ID NO: 67 - protein, 21 aa, Artificial
  signal peptide
MGSGPGVLLLLLVATAWHGQG

SEQ ID NO: 68 - DNA, 36 bp, Artificial
  Primer
cacctccatgttcttccggtaccccccagaggtaag

SEQ ID NO: 69 - protein, 8 aa, Mus musculus
DLRLYFDV

SEQ ID NO: 70 - protein, 16 aa, Mus musculus
VIWSGGGTNYNSPFMS

SEQ ID NO: 71 - protein, 10 aa, Mus musculus
GFSLTSYDIS

SEQ ID NO: 72 - protein, 8 aa, Mus musculus
GQSFTYPT

SEQ ID NO: 73 - protein, 7 aa, Mus musculus
GSSNRYT

SEQ ID NO: 74 - protein, 11 aa, Mus musculus
KASEDVGTYVS

SEQ ID NO: 75 - protein, 116 aa, Mus musculus
RVQLKESGPGLVAPSQSLSITCTVSGFSLTSYDISWIRQSPGKGLEWLGVIWSGGGTNYNSPFMSRLRISKDDSRSQVFLKVNRLQTDDTAIYYCVRDLRLYFDVWGAGTTVTVSS

SEQ ID NO: 76 - protein, 106 aa, Mus musculus
KIVMTQSPKSMSVSVGERVSLSCKASEDVGTYVSWYQQKPEQSPKLLIYGSSNRYTGVPDRFTGSGSATDFTLTISSVQAEDLADYSCGQSFTYPTFGTGTKLEIK

SEQ ID NO: 77 - protein, 8 aa, Mus musculus
DPRLYFDV

SEQ ID NO: 78 - protein, 16 aa, Mus musculus
VIWTGGGTNYNSGFMS

SEQ ID NO: 79 - protein, 10 aa, Mus musculus
GSSLDSFDIS

SEQ ID NO: 80 - protein, 8 aa, Mus musculus
GQTFSYPT

SEQ ID NO: 81 - protein, 7 aa, Mus musculus
GASNRYT

SEQ ID NO: 82 - protein, 11 aa, Mus musculus
KASEDVVTYVS

SEQ ID NO: 83 - protein, 116 aa, Mus musculus
QVQLKESGPGLVAPSKSLSITCTVSGSSLDSFDISWIRQPPGKGLEWLGVIWTGGGTNYNSGFMSRLRISKDNSKSQVFLKMSSLQSDDTAIYYCVRDPRLYFDVWGAGTTVTVSS

SEQ ID NO: 84 - protein, 106 aa, Mus musculus
NIVMTQSPKSMSMSVGERVTLSCKASEDVVTYVSWYQQKPEQSPKLLIYGASNRYTGVPDRFTGSGSATDFTLTISSIQAEDLADYYCGQTFSYPTFGTGTKLEIK

SEQ ID NO: 85 - protein, 218 aa, Artificial
  human CSF-1R fragment domains D4-D5
VVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDE

SEQ ID NO: 86 - protein, 554 aa, homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSFSPQLQESVFHLLVPSVILVLLAVGGLLFYRWRRRSHQEPQRADSPLEQPEGSPLTQDDRQVELPV

SEQ ID NO: 87 - protein, 242 aa, homo sapiens
MPRGFTWLRYLGIFLGVALGNEPLEMWPLTQNEECTVTGFLRDKLQYRSRLQYMKHYFPINYKISVPYEGVFRIANVTRLQRAQVSERELRYLWVLVSLSATESVQDVLLEGHPSWKYLQEVETLLLNVQQGLTDVEVSPKVESVLSLLNAPGPNLKLVRPKALLDNCFRVMELLYCSCCKQSSVLNWQDCEVPSPQSCSPEPSLQYAATQLYPPPPWSPSSPPHSTGSVRPVRAQGEGLLP

SEQ ID NO: 88 - protein, 126 aa, Homo sapiens
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS

SEQ ID NO: 89 - protein, 107 aa, Homo sapiens
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK

SEQ ID NO: 90 - protein, 113 aa, Artificial
  humanized S2C6 heavy chain variabel domain variant
EVQLVESGGGLVQPGGSLRLSCAASGYSFTGYYIHWVRQAPGKGLEWVARVIPNAGGTSYNQKFKGRFTLSVDNSKNTAYLQMNSLRAEDTAVYYCAREGIYWWGQGTLVTVS

SEQ ID NO: 91 - protein, 113 aa, Artificial
  humanized S2C6 light chain variabel domain variant
DIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGQGTKVEIKR

(19) United States
(12) Patent Application Publication
(10) Pub. No.: US 2017/0247459 A1
(43) Pub. Date: Aug. 31, 2017

(54) COMBINATION THERAPY OF ANTIBODIES AGAINST HUMAN CSF-1R AND USES THEREOF

(71) Applicant: HOFFMANN-LA ROCHE INC., Little Falls, NJ (US)

(72) Inventors: Michael CANNARILE, Muenchen (DE); Carola RIES, Penzberg (DE); Dominik RUETTINGER, Seehausen (DE); Katharina WARTHA, Muenchen (DE)

(73) Assignee: HOFFMANN-LA ROCHE INC., Little Falls, NJ (US)

(21) Appl. No.: 15/288,431

(22) Filed: Oct. 7, 2016

Related U.S. Application Data
(63) Continuation of application No. 13/789,373, filed on Mar. 7, 2013, now abandoned.

(30) Foreign Application Priority Data
Mar. 8, 2012 (EP) 12158519.4

(51) Int. Cl. C07K 16/28; A61K 39/395; A61K 45/06; G01N 33/574; A61K 38/20; A61K 31/513; A61K 31/519; A61K 31/4745; C07K 16/22; A61K 31/337

(52) U.S. Cl. CPC C07K 16/2866; C07K 16/2878; C07K 16/22; A61K 39/39558; A61K 45/06; A61K 31/337; A61K 38/2013; A61K 31/513; A61K 31/519; A61K 31/4745; G01N 33/57492; G01N 33/57488; C07K 2317/31; C07K 2317/56; C07K 2317/24; C07K 2317/75; C07K 2317/76; C07K 2317/73; C07K 2317/92; A61K 2039/505

(57) ABSTRACT

The present invention relates to the combination therapy of antibodies binding to human CSF-1R, characterized in binding to the (dimerization) domains D4 to D5 (SEQ ID No: 85) of the extracellular domain of human CSF-1R in combination with a chemotherapeutic agent, radiation, and/or cancer immunotherapy.

### RELATED APPLICATIONS

This application is a continuation of U.S. application Ser. No. 13/789,373, filed Mar. 7, 2013, which claims the benefit of European Patent Application No. 12 158 519.4, filed on Mar. 8, 2012, the disclosures of which are hereby incorporated by reference in their entireties for all purposes.

### SEQUENCE LISTING

The content of the following submission on ASCII text file is incorporated herein by reference in its entirety: a computer readable form (CRF) of the Sequence Listing (file name: 146392029901SeqList.txt, date recorded: Oct. 6, 2016, size: 80 KB).

### FIELD OF THE INVENTION

The present invention relates to anti-CSF-1R which bind to human CSF-1R. The antibodies of the invention specifically bind to the (dimerization) domains D4 to D5 and can be administered with therapy comprising a chemotherapeutic agent, radiation, cancer immunotherapy, and combinations thereof.

### BACKGROUND OF THE INVENTION

The human CSF-1 receptor (CSF-1R; colony stimulating factor 1 receptor; synonyms: M-CSF receptor; Macrophage colony-stimulating factor 1 receptor, Fms proto-oncogene, c-fms, SEQ ID NO: 62) is known since 1986 (Coussens, L., et al., Nature 320 (1986) 277-280). CSF-1R is a growth factor and encoded by the c-fms proto-oncogene (reviewed e.g. in Roth, P., and Stanley, E. R., Curr. Top. Microbiol. Immunol. 181 (1992) 141-167).

CSF-1R is the receptor for CSF-1 (colony stimulating factor 1, also called M-CSF, macrophage colony-stimulating factor) and mediates the biological effects of this cytokine (Sherr, C. J., et al., Cell 41 (1985) 665-676). The cloning of the colony stimulating factor-1 receptor (CSF-1R) (also called c-fms) was described for the first time in Roussel, M. F., et al., Nature 325 (1987) 549-552. In that publication, it was shown that CSF-1R had transforming potential dependent on changes in the C-terminal tail of the protein including the loss of the inhibitory tyrosine 969 phosphorylation which binds Cbl and thereby regulates receptor down regulation (Lee, P. S., et al., Embo J. 18 (1999) 3616-3628). Recently a second ligand for CSF-1R termed interleukin-34 (IL-34) was identified (Lin, H., et al, Science 320 (2008) 807-811).

Currently two CSF-1R ligands that bind to the extracellular domain of CSF-1R are known. The first one is CSF-1 (colony stimulating factor 1, also called M-CSF, macrophage; SEQ ID NO: 86) and is found extracellularly as a disulfide-linked homodimer (Stanley, E. R. et al., Journal of Cellular Biochemistry 21 (1983) 151-159; Stanley, E. R. et al., Stem Cells 12 Suppl. 1 (1995) 15-24). The second one is IL-34 (Human IL-34; SEQ ID NO: 87) (Hume, D. A., et al, Blood 119 (2012) 1810-1820). The main biological effects of CSF-1R signaling are the differentiation, proliferation, migration, and survival of hematopoietic precursor cells to the macrophage lineage (including osteoclast). Activation of CSF-1R is mediated by its CSF-1R ligands, CSF-1 (M-CSF) and IL-34. Binding of CSF-1 (M-CSF) to CSF-1R induces the formation of homodimers and activation of the kinase by tyrosine phosphorylation (Li, W. et al, EMBO Journal. 10 (1991) 277-288; Stanley, E. R., et al., Mol. Reprod. Dev. 46 (1997) 4-10). The biologically active homodimer CSF-1 binds to the CSF-1R within the subdomains D1 to D3 of the extracellular domain of the CSF-1 receptor (CSF-1R-ECD). The CSF-1R-ECD comprises five immunoglobulin-like subdomains (designated D1 to D5). The subdomains D4 to D5 of the extracellular domain (CSF-1R-ECD) are not involved in the CSF-1 binding (Wang, Z., et al Molecular and Cellular Biology 13 (1993) 5348-5359). The subdomain D4 is involved in dimerization (Yeung, Y-G., et al Molecular & Cellular Proteomics 2 (2003) 1143-1155; Pixley, F. J., et al., Trends Cell Biol 14 (2004) 628-638).

Further signaling is mediated by the p85 subunit of PI3K and Grb2 connecting to the PI3K/AKT and Ras/MAPK pathways, respectively. These two important signaling pathways can regulate proliferation, survival and apoptosis. Other signaling molecules that bind the phosphorylated intracellular domain of CSF-1R include STAT1, STAT3, PLCy, and Cbl (Bourette, R. P. and Rohrschneider, L. R., Growth Factors 17 (2000) 155-166).

CSF-1R signaling has a physiological role in immune responses, in bone remodeling and in the reproductive system. The knockout animals for either CSF-1 (Pollard, J. W., Mol. Reprod. Dev. 46 (1997) 54-61) or CSF-1R (Dai, X. M., et al., Blood 99 (2002) 111-120) have been shown to have osteopetrotic, hematopoietic, tissue macrophage, and reproductive phenotypes consistent with a role for CSF-1R in the respective cell types.

Sherr, C. J., et al., Blood 73 (1989) 1786-1793 relates to some antibodies against CSF-1R that inhibit the CSF-1 activity. Ashmun, R. A., et al., Blood 73 (1989) 827-837 relates to CSF-1R antibodies. Lenda, D., et al., Journal of Immunology 170 (2003) 3254-3262 relates to reduced macrophage recruitment, proliferation, and activation in CSF-1-deficient mice results in decreased tubular apoptosis during renal inflammation. Kitaura, H., et al., Journal of Dental Research 87 (2008) 396-400 refers to an anti-CSF-1 antibody which inhibits orthodontic tooth movement. WO 2001/030381 mentions CSF-1 activity inhibitors including antisense nucleotides and antibodies while disclosing only CSF-1 antisense nucleotides. WO 2004/045532 relates to metastases and bone loss prevention and treatment of metastatic cancer by a CSF-1 antagonist disclosing as antagonist anti-CSF-1-antibodies only. WO 2005/046657 relates to the treatment of inflammatory bowel disease by anti-CSF-1-antibodies. US 2002/0141994 relates to inhibitors of colony stimulating factors. WO 2006/096489 relates to the treatment of rheumatoid arthritis by anti-CSF-1-antibodies. WO 2009/026303 and WO 2009/112245 relate to certain anti-CSF-1R antibodies binding to CSF-1R within the first three subdomains (D1 to D3) of the Extracellular Domain (CSF-1R-ECD). WO2011/123381(A1) relates to antibodies against CSF-1R.

### SUMMARY OF THE INVENTION

The inventions provides anti-CSF-1R antibodies and methods of treatment using such antibodies.

One embodiment of the invention provides an anti-CSF-1R antibody comprising

a) a heavy chain variable domain comprising SEQ ID NO:7 and the light chain variable domain comprising SEQ ID NO:8,
b) a heavy chain variable domain comprising SEQ ID NO:15 and the light chain variable domain comprising SEQ ID NO:16;
c) a heavy chain variable domain comprising SEQ ID NO:75 and the light chain variable domain comprising SEQ ID NO:76;
d) a heavy chain variable domain comprising SEQ ID NO:83 and the light chain variable domain comprising SEQ ID NO:84. In some embodiments, the antibodies are humanized. In some embodiments, the antibody is an IgG1. In some embodiments, the antibody is an IgG4.

Another embodiment of the invention provides anti-CSF-1R antibodies comprising

a) a heavy chain variable domain comprising a CDR3 region of SEQ ID NO: 1, a CDR2 region of SEQ ID NO: 2, and a CDR1 region of SEQ ID NO:3, and a light chain variable domain comprising a CDR3 region of SEQ ID NO: 4, a CDR2 region of SEQ ID NO:5, and a CDR1 region of SEQ ID NO:6, or
b) a heavy chain variable domain comprising a CDR3 region of SEQ ID NO: 9, a CDR2 region of SEQ ID NO: 10, and a CDR1 region of SEQ ID NO: 11, and a light chain variable domain comprising a CDR3 region of SEQ ID NO:12, a CDR2 region of SEQ ID NO: 13, and a CDR1 region of SEQ ID NO: 14, or
c) a heavy chain variable domain comprising a CDR3 region of SEQ ID NO: 17, a CDR2 region of SEQ ID NO: 18, and a CDR1 region of SEQ ID NO:19, and a light chain variable domain comprising a CDR3 region of SEQ ID NO: 20, a CDR2 region of SEQ ID NO:21, and a CDR1 region of SEQ ID NO:22, or
d) a heavy chain variable domain comprising a CDR3 region of SEQ ID NO: 25, a CDR2 region of SEQ ID NO: 26, and a CDR1 region of SEQ ID NO: 27, and a light chain variable domain comprising a CDR3 region of SEQ ID NO:28, a CDR2 region of SEQ ID NO: 29, and a CDR1 region of SEQ ID NO: 30, or
e) a heavy chain variable domain comprising a CDR3 region of SEQ ID NO: 33, a CDR2 region of SEQ ID NO: 34, and a CDR1 region of SEQ ID NO: 35, and a light chain variable domain comprising a CDR3 region of SEQ ID NO:36, a CDR2 region of SEQ ID NO: 37, and a CDR1 region of SEQ ID NO: 38, or
f) a heavy chain variable domain comprising a CDR3 region of SEQ ID NO:41, a CDR2 region of SEQ ID NO: 42, and a CDR1 region of SEQ ID NO:43, and a light chain variable domain comprising a CDR3 region of SEQ ID NO: 44, a CDR2 region of SEQ ID NO:45, and a CDR1 region of SEQ ID NO:46, or
g) a heavy chain variable domain comprising a CDR3 region of SEQ ID NO: 49, a CDR2 region of SEQ ID NO: 50, and a CDR1 region of SEQ ID NO: 51, and a light chain variable domain comprising a CDR3 region of SEQ ID NO:52, a CDR2 region of SEQ ID NO: 53, and a CDR1 region of SEQ ID NO: 54; or
h) a heavy chain variable domain comprising a CDR3 region of SEQ ID NO:69, a CDR2 region of SEQ ID NO: 70, and a CDR1 region of SEQ ID NO:71, and a light chain variable domain comprising a CDR3 region of SEQ ID NO: 72, a CDR2 region of SEQ ID NO:73, and a CDR1 region of SEQ ID NO:74, or
i) a heavy chain variable domain comprising a CDR3 region of SEQ ID NO: 77, a CDR2 region of SEQ ID NO: 78, and a CDR1 region of SEQ ID NO: 79, and a light chain variable domain comprising a CDR3 region of SEQ ID NO:80, a CDR2 region of SEQ ID NO: 81, and a CDR1 region of SEQ ID NO: 82. In some embodiments, the antibodies are humanized. In some embodiments, the antibody is an IgG1. In some embodiments, the antibody is an IgG4.

One embodiment of the invention provide methods of inhibiting

a) proliferation of CSF-1R ligand-dependent and/or CSF-1R ligand-independent CSF-1R expressing tumor cells;
b) proliferation of tumors with CSF-1 ligand-dependent and/or CSF-1 ligand-dependent independent CSF-1R expressing macrophage infiltrate;
c) cell survival (in CSF-1R ligand-dependent and/or CSF-1R ligand-independent) CSF-1R expressing monocytes and macrophages;
d) cell differentiation (in CSF-1R ligand-dependent and/or CSF-1R ligand-independent) CSF-1R expressing monocytes into macrophages; or
e) a combination thereof. The methods comprise administering to a patient an anti-CSF-1R antibody that specifically binds to the (dimerization) domains D4 to D5 (SEQ ID No: 85) of the extracellular domain of human CSF-1R in combination with a chemotherapeutic agent, radiation, cancerimmunotherapy, and combinations thereof.

Another embodiment of the invention provides methods of treating a patient having a CSF-1R expressing tumor or having a tumor with CSF-1R expressing. macrophage infiltrate, wherein the tumor has an increase of CSF-1R ligand. The methods comprise administering a therapy comprising an effective amount of an anti-CSF-1R antibody that specifically binds to the domains D4 to D5 (SEQ ID No: 85) of the extracellular domain of human CSF-1R, and a chemotherapeutic agent, radiation, cancer immunotherapy, and combinations thereof. In some embodiments, the chemotherapeutic agent is selected from taxanes (paclitaxel (Taxol), docetaxel (Taxotere), modified paclitaxel (Abraxane and Opaxio)), doxorubicin, modified doxorubicin (Caelyx or Doxil)), sunitinib (Sutent), sorafenib (Nexavar), and other multikinase inhibitors, oxaliplatin, cisplatin, carboplatin, etoposide, gemcitabine, and vinblastine. In some embodiments, the cancer immunotherapy is selected from:

a) T cell engaging agents selected from agonistic antibodies which bind to human OX40, TO GITR, TO CD27, OR TO 4-1BB, and T-cell bispecific antibodies (e.g. T cell-engaging BiTE™ antibodies CD3-CD19, CD3-EpCam, CD3-EGFR), IL-2 (Proleukin), Interferon (IFN) alpha, antagonizing antibodies which bind to human CTLA-4, to PD-1, to PD-L1, to TIM-3, to BTLA, to VISTA, to LAG-3, or to CD25,
b) targeting immunosuppression: antibodies or small molecules targeting STATS or NFkB signaling, blocking IL-6, IL-17, IL-23, TNFa function,
c) cancer vaccines/enhance dendritic cell function: oncolytic virus secreting GM-CSF (OncoVex), an agonistic CD40 antibody, Toll-like receptor (TLR) ligands, TLR agonists, recombinant fusion protein encoding MAGE-A3, PROSTVAC; or
d) adoptive cell transfer: GVAX (prostate cancer cell line expressing GM-CSF), dendritic cell vaccine, adoptive T cell therapy, adoptive CAR T cell therapy.

In some embodiments, the cancer immunotherapy is an agonistic CD40 antibody.

In some embodiments, the chemotherapeutic agent is selected from taxanes (docetaxel or paclitaxel or a modified paclitaxel (Abraxane or Opaxio)), doxorubicin, capecitabine. bevacizumab, and combinations thereof and the patient has been diagnosed with breast cancer. In some embodiments, the chemotherapeutic agent is selected from carboplatin, oxaliplatin, cisplatin, paclitaxel, doxorubicin (or modified doxorubicin (Caelyx or Doxil)), topotecan (Hycamtin), and combinations thereof and further wherein the patient has been diagnosed with ovarian cancer. In some embodiments, the chemotherapeutic agent is selected from multi-kinase inhibitor (sunitinib (Sutent), sorafenib (Nexavar) or motesanib diphosphate (AMG 706), doxorubicin, and combinations thereof and further wherein the patient has been diagnosed with renal cancer. In some embodiments, the chemotherapeutic agent is selected from oxaliplatin, cisplatin, radiation, and combinations thereof and the patient has been diagnosed with squamous cell carcinoma. In some embodiments, the chemotherapeutic agent is selected from taxol, carboplatin, and combinations thereof and the patient has been diagnosed with lung cancer. In some embodiments, the antibody does not bind to human CSF-1R fragment delD4 (SEQ ID NO: 65). In some embodiments, the antibody binds to human CSF-1R fragment delD4 (SEQ ID NO: 65) and to human CSF-1R Extracellular Domain (SEQ ID NO: 64) with a ratio of 1:50 or lower. In some embodiments, the antibody comprises

a) a heavy chain variable domain comprising SEQ ID NO:7 and the light chain variable domain comprising SEQ ID NO:8,
b) a heavy chain variable domain comprising SEQ ID NO:15 and the light chain variable domain comprising SEQ ID NO:16;
c) a heavy chain variable domain comprising SEQ ID NO:75 and the light chain variable domain comprising SEQ ID NO:76;
d) a heavy chain variable domain comprising SEQ ID NO:83 and the light chain variable domain comprising SEQ ID NO:84;
or a humanized version thereof. In some embodiments, the antibody comprises
a) a heavy chain variable domain comprising SEQ ID NO:23 and the light chain variable domain comprising SEQ ID NO:24, or
b) a heavy chain variable domain comprising SEQ ID NO:31 and the light chain variable domain comprising SEQ ID NO:32, or
c) a heavy chain variable domain comprising SEQ ID NO:39 and the light chain variable domain comprising SEQ ID NO:40, or
d) a heavy chain variable domain comprising SEQ ID NO:47 and the light chain variable domain comprising SEQ ID NO:48, or
e) a heavy chain variable domain comprising SEQ ID NO:55 and the light chain variable domain comprising SEQ ID NO:56.

In some embodiments, the antibody comprises

a) a heavy chain variable domain comprising a CDR3 region of SEQ ID NO: 1, a CDR2 region of SEQ ID NO: 2, and a CDR1 region of SEQ ID NO:3, and a light chain variable domain comprising a CDR3 region of SEQ ID NO: 4, a CDR2 region of SEQ ID NO:5, and a CDR1 region of SEQ ID NO:6, or
b) a heavy chain variable domain comprising a CDR3 region of SEQ ID NO: 9, a CDR2 region of SEQ ID NO: 10, and a CDR1 region of SEQ ID NO: 11, and a light chain variable domain comprising a CDR3 region of SEQ ID NO:12, a CDR2 region of SEQ ID NO: 13, and a CDR1 region of SEQ ID NO: 14, or
c) a heavy chain variable domain comprising a CDR3 region of SEQ ID NO: 17, a CDR2 region of SEQ ID NO: 18, and a CDR1 region of SEQ ID NO:19, and a light chain variable domain comprising a CDR3 region of SEQ ID NO: 20, a CDR2 region of SEQ ID NO:21, and a CDR1 region of SEQ ID NO:22, or
d) a heavy chain variable domain comprising a CDR3 region of SEQ ID NO: 25, a CDR2 region of SEQ ID NO: 26, and a CDR1 region of SEQ ID NO: 27, and a light chain variable domain comprising a CDR3 region of SEQ ID NO:28, a CDR2 region of SEQ ID NO: 29, and a CDR1 region of SEQ ID NO: 30, or
e) a heavy chain variable domain comprising a CDR3 region of SEQ ID NO: 33, a CDR2 region of SEQ ID NO: 34, and a CDR1 region of SEQ ID NO: 35, and a light chain variable domain comprising a CDR3 region of SEQ ID NO:36, a CDR2 region of SEQ ID NO: 37, and a CDR1 region of SEQ ID NO: 38, or
f) a heavy chain variable domain comprising a CDR3 region of SEQ ID NO:41, a CDR2 region of SEQ ID NO: 42, and a CDR1 region of SEQ ID NO:43, and a light chain variable domain comprising a CDR3 region of SEQ ID NO: 44, a CDR2 region of SEQ ID NO:45, and a CDR1 region of SEQ ID NO:46, or
g) a heavy chain variable domain comprising a CDR3 region of SEQ ID NO: 49, a CDR2 region of SEQ ID NO: 50, and a CDR1 region of SEQ ID NO: 51, and a light chain variable domain comprising a CDR3 region of SEQ ID NO:52, a CDR2 region of SEQ ID NO: 53, and a CDR1 region of SEQ ID NO: 54; or
h) a heavy chain variable domain comprising a CDR3 region of SEQ ID NO:69, a CDR2 region of SEQ ID NO: 70, and a CDR1 region of SEQ ID NO:71, and a light chain variable domain comprising a CDR3 region of SEQ ID NO: 72, a CDR2 region of SEQ ID NO:73, and a CDR1 region of SEQ ID NO:74, or
i) a heavy chain variable domain comprising a CDR3 region of SEQ ID NO: 77, a CDR2 region of SEQ ID NO: 78, and a CDR1 region of SEQ ID NO: 79, and a light chain variable domain comprising a CDR3 region of SEQ ID NO:80, a CDR2 region of SEQ ID NO: 81, and a CDR1 region of SEQ ID NO: 82. In some embodiments, the antibody is a human IgG1 or a human IgG4.

A further embodiment of the invention provides methods for treating a patient having a CSF-1R expressing tumor or having a tumor with CSF-1R expressing macrophage infiltrate, wherein the tumor is characterized by an increase of CSF-1R ligand, the method comprising administering an antibody that specifically binds to human CSF-1R and a cancer immunotherapy. In some embodiments, the cancer immunotherapy is selected from:

a) T cell engaging agents selected from agonistic antibodies which bind to human OX40, to GITR, to CD27, or to 4-1BB, and T-cell bispecific antibodies (e.g. T cell-engaging BiTE™ antibodies CD3-CD19, CD3-EpCam, CD3-EGFR), IL-2 (Proleukin), Interferon (IFN) alpha, antagonizing antibodies which bind to human CTLA-4 (e.g. ipilimumab), to PD-1, to PD-L1, to TIM-3, to BTLA, to VISTA, to LAG-3, or to CD25,
b) targeting immunosuppression: antibodies or small molecules targeting STATS or NFkB signaling, blocking IL-6, IL-17, IL-23, TNFa function,
c) cancer vaccines/enhance dendritic cell function: OncoVex (oncolytic virus secreting GM-CSF), an agonistic CD40 antibody, Toll-like receptor (TLR) ligands, TLR agonists, recombinant fusion protein encoding MAGE-A3, PROSTVAC; or
d) adoptive cell transfer: GVAX (prostate cancer cell line expressing GM-CSF), dendritic cell vaccine, adoptive T cell therapy, adoptive CAR T cell therapy. In some embodiments, the cancer immunotherapy is selected from: cancer vaccines/enhance dendritic cell function: OncoVex (oncolytic virus secreting GM-CSF), an agonistic CD40 antibody, Toll-like receptor (TLR) ligands, TLR agonists, recombinant fusion protein encoding MAGE-A3, PROSTVAC. In some embodiments, the cancer immunotherapy is an agonistic CD40 antibody. Yet another embodiment of the invention provides methods for determining whether a subject having a cancer is a candidate for an anti-CSF-1R antibody-based cancer treatment regimen. The methods comprise—ex vivo or in vitro determining in vitro the level of one or more of the following markers: CSF-1R, CD68/CD163, CD68/MHC class II, CD31 (microvessel density), and Ki67 and other markers like e g immuninfiltrates; in a sample of the subject, wherein the sample is selected from tissue, blood, serum, plasma, tumor cells and circulating tumor cells; and wherein a change in the level of one or more of CSF-1R, CD68/CD163, CD68/MHC class II, CD31 (microvessel density) and Ki67 and other markers like e.g. immuninfiltrates (e.g. T cells (e.g. CD4- and/or CD8-T cells), as compared with to the corresponding level in an individual not suffering from cancer, is indicative that the subject is a candidate for the anti-CSF-1 R antibody-based cancer treatment regimen. The anti-CSF-1R antibody may be any anti-CSF-1R antibody described herein. In some embodiments, the change in the level of CSF-1R, CD68/CD163, CD68/MHC class II, CD31 (microvessel density) and Ki67 and other markers like e.g. immuninfiltrates (e.g. T cells (e.g. CD4- and/or CD8-T cells), as compared to the level in an individual not suffering from cancer is an increase in the level of one or more of these markers.

Even another embodiment of the invention provides methods for determining whether a subject having a cancer is a candidate for a therapy comprising an anti-CSF-1R antibody. The methods comprise ex vivo or in vitro determining in vitro the level of one or more of the following markers: CSF-1, Trap5b, sCD163, IL-34; in a sample of the subject, wherein the sample is selected from tissue, blood, serum, plasma, tumor cells and circulating tumor cells; and wherein a change in the level of one or more of CSF-1, Trap5b, sCD163, IL-34, as compared with to the corresponding level in an individual not suffering from cancer, is indicative that the subject is a candidate for the therapy. The anti-CSF-1R antibody may be any anti-CSF-1R antibody described herein. In some embodiments, the change in the level of CSF-1, Trap5b, sCD163, IL-34, as compared to the level in an individual not suffering from cancer is an increase in the level of one or more of these markers. In some embodiments, the ex vivo or in vitro the level and change of the level of sCD163 is determined.

A further embodiment of the invention provides methods for determining whether a subject having a cancer is a candidate for a therapy comprising an anti-CSF-1R antibody. The methods comprise ex vivo or in vitro determining in vitro the level of one or more of the following markers: IFNγ, TNFα, IL-1β, IL-4, IL-6, IL-8, IL-10, IL-13, GM-CSF, VEGF, MCP-1, CCL18, CCL22, MIP-1, Galectin 3, IL1Ra, TGF alpha; in a sample of the subject, wherein the sample is selected from tissue, blood, serum, plasma, tumor cells and circulating tumor cells; and wherein a change in the level of one or more of IFNγ, TNFα, IL-1β, IL-4, IL-6, IL-8, IL-10, IL-13, GM-CSF, VEGF, MCP-1, CCL18, CCL22, MIP-1, Galectin 3, IL1Ra, TGF alpha, as compared with to the corresponding level in an individual not suffering from cancer, is indicative that the subject is a candidate for the therapy. The anti-CSF-1R antibody may be any anti-CSF-1R antibody described herein. In some embodiments, the change in the level of IFNγ, TNFα, IL-1β, IL-4, IL-6, IL-8, IL-10, IL-13, GM-CSF, VEGF, MCP-1, CCL18, CCL22, MIP-1, Galectin 3, IL1Ra, TGF alpha, as compared to the level in an individual not suffering from cancer is an increase in the level of one or more of these markers. In some embodiments, the invention provides methods of treating cancer, comprising administering therapy comprising an anti-CSF-1R antibody and a bispecific ANG-2-VEGF antibody. In other embodiments, the invention provides methods of treating cancer, comprising administering therapy comprising an anti-CSF-1R antibody is and an agonistic CD40 antibody. In some embodiments, i) the anti-CSF-1R antibody comprises (a) a heavy chain variable domain amino acid sequence of SEQ ID NO:39 and (b) a light chain variable domain amino acid sequence of SEQ ID NO:40; and ii) the agonistic CD40 antibody comprises (a) a heavy chain variable domain amino acid sequence of SEQ ID NO: 88 and (b) a light chain variable domain amino acid sequence of SEQ ID NO: 89. In some embodiments, the anti-CSF-1R antibody comprises (a) a heavy chain variable domain amino acid sequence of SEQ ID NO:39 and (b) a light chain variable domain amino acid sequence of SEQ ID NO:40; and the agonistic CD40 antibody is dacetuzumab. In some embodiments, i) the anti-CSF-1R antibody comprises (a) a heavy chain variable domain amino acid sequence of SEQ ID NO:39 and (b) a light chain variable domain amino acid sequence of SEQ ID NO:40; and ii) the agonistic CD40 antibody comprises (a) a heavy chain variable domain amino acid sequence of SEQ ID NO: 90 and (b) a light chain variable domain amino acid sequence of SEQ ID NO: 91.)

One embodiment of the invention provides an antibody binding to human CSF-1R, characterized in binding to the (dimerization) domains D4 to D5 (SEQ ID No: 85) of the extracellular domain of human CSF-1R for use in
a) the inhibition of cell proliferation in CSF-1R ligand-dependent and/or CSF-1 ligand-independent CSF-1R expressing tumor cells;
b) the inhibition of cell proliferation of tumors with CSF-1R ligand-dependent and/or CSF-1R ligand-independent CSF-1R expressing macrophage infiltrate;
c) the inhibition of cell survival (in CSF-1R ligand-dependent and/or CSF-1R ligand-independent) CSF-1R expressing monocytes and macrophages; and/or
d) the inhibition of cell differentiation (in CSF-1R ligand-dependent and/or CSF-1R ligand-independent) CSF-1R expressing monocytes into macrophages,
   wherein the anti-CSF-1R antibody is administered in combination with a chemotherapeutic agent, radiation, and/or cancer immunotherapy.

This combination therapy with antibodies binding to human CSF-1R, characterized in binding to the (dimerization) domains D4 to D5, has valuable properties like less activation potential to CSF-1R activation and in consequence reduced toxicity and no stimulation of CSF-1R receptor (e.g. compared to a combination therapy with antibodies binding to human CSF-1R, characterized in binding to the domains D1 to D3).

The term “ligand dependent” as used herein refers to a ligand-independent signaling through the extracellular ECD (and does not include the ligand independent signaling mediated by activating point mutations in the intracellular kinase domain) In one embodiment CSF-1R ligand in this context refers a CSF-1R ligand selected from human CSF-1 (SEQ ID No: 86) and human IL-34 (SEQ ID No: 87); in one embodiment the CSF-1R ligand is human CSF-1 (SEQ ID No: 86); in one embodiment the CSF-1R ligand is human IL-34 (SEQ ID No: 87)). The invention comprises an antibody binding to human CSF-1R, antibody binding to human CSF-1R, characterized in binding to the (dimerization) domains D4 to D5 (SEQ ID No: 85) of the extracellular domain of human CSF-1R for use in the treatment of a patient having a CSF-1R expressing tumor or having a tumor with CSF-1R expressing macrophage infiltrate, wherein the tumor is characterized by an increase of CSF-1R ligand (in one embodiment the CSF-1R ligand is selected from human CSF-1 (SEQ ID No: 86) and human IL-34 (SEQ ID No: 87); in one embodiment the CSF-1R ligand is human CSF-1 (SEQ ID No: 86); in one embodiment the CSF-1R ligand is human IL-34 (SEQ ID No: 87)) (detectable in serum, urine or tumor biopsies),

wherein the anti-CSF-1R antibody is administered in combination with a chemotherapeutic agent, radiation and/or cancer immunotherapy. The term “increase of CSF-1R ligand” refers to the overexpression of human CSF-1R ligand (in one embodiment the CSF-1R ligand is selected from human CSF-1 (SEQ ID No: 86) and human IL-34 (SEQ ID No: 87); in one embodiment the CSF-1R ligand is human CSF-1 (SEQ ID No: 86); in one embodiment the CSF-1R ligand is human IL-34 (SEQ ID No: 87)) (compared to normal tissue) before treatment or overexpression of human CSF-1R ligand induced by treatment with anti-CSF-1R antibody (and compared to the expression levels before treatment).

In certain embodiments, the term “increase” or “above” refers to a level above the reference level or to an overall increase of 5%, 10%, 20%, 25%, 30%, 40%, 50%, 60%, 70%, 80%, 85%, 90%, 95%, 100% or greater, in CSF-1R ligand level detected by the methods described herein, as compared to the CSF-1R ligand level from a reference sample (e.g., normal tissue). In certain embodiments, the term increase refers to the increase in CSF-1R ligand level wherein, the increase is at least about 1.5-, 1.75-, 2-, 3-, 4-, 5-, 6-, 7-, 8-, 9-, 10-, 15-, 20-, 25-, 30-, 40-, 50-, 60-, 70-, 75-, 80-, 90-, or 100-fold higher as compared to the CSF-1R ligand level e.g. predetermined from a reference sample. In one preferred embodiment the term increased level relates to a value at or above a reference level (e.g., a level in normal tissue).

In one embodiment of the invention the anti-CSF-1R antibody is characterized in that the antibody binds to human CSF-1R Extracellular Domain (SEQ ID NO: 64) (comprising domains D1 to D5) and does not bind to domains D1 to D3 (SEQ ID NO: 66) of the extracellular domain of human CSF-1R.

In one embodiment chemotherapeutic agents, which may be administered with anti-CSF-1R antibody, include, but are not limited to, anti-neoplastic agents including alkylating agents including: nitrogen mustards, such as mechlorethamine, cyclophosphamide, ifosfamide, melphalan and chlorambucil; nitrosoureas, such as carmustine (BCNU), lomustine (CCNU), and semustine (methyl-CCNU); Temodal™ (temozolamide), ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil (5FU), fluorodeoxyuridine, gemcitabine, cytosine arabinoside (AraC, cytarabine), 5-azacytidine, 2,2′-difluorodeoxycytidine, purine analogs such as 6-merca.rho.topurine, 6-thioguamne, azathioprine, T-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; pipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycinC, and actinomycin; enzymes such as L-asparaginase; biological response modifiers such as interferon-alpha, IL-2, G-CSF and GM-CSF; miscellaneous agents including platinum coordination complexes such as oxaliplatin, cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o, p-DDD) and aminoglutethimide; hormones and antagonists including adrenocorticosteroid antagonists such as prednisone and equivalents, dexamethasone and aminoglutethimide; Gemzar™ (gemcitabine), progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide. Therapies targeting epigenetic mechanism including, but not limited to, histone deacetylase inhibitors, demethylating agents (e.g., Vidaza) and release of transcriptional repression (ATRA) therapies can also be combined with the antigen binding proteins.

In one embodiment the chemotherapeutic agent is selected from the group consisting of taxanes (e.g. paclitaxel (Taxol), docetaxel (Taxotere), modified paclitaxel (e.g., Abraxane and Opaxio), doxorubicin, sunitinib (Sutent), sorafenib (Nexavar), and other multikinase inhibitors, oxaliplatin, cisplatin and carboplatin, etoposide, gemcitabine, and vinblastine. In one embodiment the chemotherapeutic agent is selected from the group consisting of taxanes (like e.g. taxol (paclitaxel), docetaxel (Taxotere), modified paclitaxel (e.g. Abraxane and Opaxio).

In one embodiment the chemotherapeutic agent is selected from 5-fluorouracil(5-FU), leucovorin, irinotecan, or oxaliplatin. In one embodiment the chemotherapeutic agent is 5-fluorouracil, leucovorin and irinotecan (FOLFIRI). In one embodiment the chemotherapeutic agent is 5-fluorouracil, and oxaliplatin (FOLFOX).

Specific examples of combination therapies with chemotherapeutic agents include, for instance, an CSF-1R antibody with taxanes (e.g., docetaxel or paclitaxel) or a modified paclitaxel (e.g., Abraxane or Opaxio), doxorubicin), capecitabine and/or bevacizumab (Avastin) for the treatment of breast cancer; the human CSF-1R antibody with carboplatin, oxaliplatin, cisplatin, paclitaxel, doxorubicin (or modified doxorubicin (Caelyx or Doxil)), or topotecan (Hycamtin) for ovarian cancer, the human CSF-1R antibody with a multi-kinase inhibitor, MM, (Sutent, Nexavar, or 706) and/or doxorubicin for treatment of kidney (i.e., renal) cancer; the CSF-1R antibody with oxaliplatin, cisplatin and/or radiation for the treatment of squamous cell carcinoma; the CSF-1R antibody with taxol and/or carboplatin for the treatment of lung cancer.

Therefore, in one embodiment the chemotherapeutic agent is selected from the group of taxanes (docetaxel or paclitaxel or a modified paclitaxel (Abraxane or Opaxio), doxorubicin, capecitabine and/or bevacizumab for the treatment of breast cancer.

In one embodiment the chemotherapeutic agent is selected from the group of carboplatin, oxaliplatin, cisplatin, paclitaxel, doxorubicin (or modified doxorubicin (Caelyx or Doxil)), or topotecan (Hycamtin) for the treatment of ovarian cancer. In one embodiment the chemotherapeutic agent is selected from the group of a multi-kinase inhibitor (sunitinib (Sutent), sorafenib (Nexavar) or motesanib diphosphate (AMG 706) and/or doxorubicin for treatment of kidney (i.e., renal) cancer.

In one embodiment the chemotherapeutic agent is selected from the group of oxaliplatin, cisplatin and/or radiation for the treatment of squamous cell carcinoma.

In one embodiment the chemotherapeutic agent is selected from the group of taxol and/or carboplatin for the treatment of lung cancer.

In one embodiment cancer immunotherapy, which may be administered with anti-CSF-1R antibody, includes, but is not limited to, activating T cells or inhibiting Treg cells, activating antigen presenting cells, inhibiting immunosuppressive cells in the tumor microenvironment, cancer vaccines and adoptive cell transfer, T cell engaging agent.

In one embodiment the cancer immunotherapy is selected from the group of:
a) T cell engaging agents selected from agonistic antibodies which bind to human OX40, TO GITR, TO CD27, OR TO 4-1BB, and T-cell bispecific antibodies (e.g. T cell-engaging BiTE™ antibodies CD3-CD19, CD3-EpCam, CD3-EGFR), IL-2 (Proleukin), Interferon (IFN) alpha, antagonizing antibodies which bind to human CTLA-4 (e.g. ipilimumab), to PD-1, to PD-L1, to TIM-3, to BTLA, to VISTA, to LAG-3, or to CD25,
b) targeting immunosuppression: antibodies or small molecules targeting STATS or NFkB signaling, blocking IL-6, IL-17, IL-23, TNFa function,
c) cancer vaccines/enhance dendritic cell function: OncoVex (oncolytic virus secreting GM-CSF), an agonistic CD40 antibody, Toll-like receptor (TLR) ligands, TLR agonists, recombinant fusion protein encoding MAGE-A3, PROSTVAC; or
d) adoptive cell transfer: GVAX (prostate cancer cell line expressing GM-CSF), dendritic cell vaccine, adoptive T cell therapy, adoptive CAR T cell therapy.

In one embodiment the cancer immunotherapy is selected from T cell engaging agents selected from IL-2 (Proleukin), and antagonizing antibodies which bind to human CTLA-4 (e.g. ipilimumab), to PD-1, or to PD-L1.

In one embodiment the cancer immunotherapy is IL-2 (Proleukin). In one embodiment the cancer immunotherapy is an antagonizing antibody which binds to human CTLA-4 (e.g. ipilimumab).

One further aspect of the invention is the combination therapy of an antibody binding to human CSF-1R (including antibodies binding to domains D1-D3 and antibodies binding to domains D4-D5) with a cancer immunotherapy, wherein the cancer immunotherapy is selected from the group of:
a) T cell engaging agents selected from agonistic antibodies which bind to human OX40, to GITR, to CD27, or to 4-1BB, and T-cell bispecific antibodies (e.g. T cell-engaging BiTE™ antibodies CD3-CD19, CD3-EpCam, CD3-EGFR), IL-2 (Proleukin), Interferon (IFN) alpha, antagonizing antibodies which bind to human CTLA-4 (e.g. ipilimumab), to PD-1, to PD-L1, to TIM-3, to BTLA, to VISTA, to LAG-3, or to CD25,
b) targeting immunosuppression: antibodies or small molecules targeting STATS or NFkB signaling, blocking IL-6, IL-17, IL-23, TNFa function,
c) cancer vaccines/enhance dendritic cell function: OncoVex (oncolytic virus secreting GM-CSF), an agonistic CD40 antibody, Toll-like receptor (TLR) ligands, TLR agonists, recombinant fusion protein encoding MAGE-A3, PROSTVAC; or
d) adoptive cell transfer: GVAX (prostate cancer cell line expressing GM-CSF), dendritic cell vaccine, adoptive T cell therapy, adoptive CAR T cell therapy.
One further aspect of the invention is the combination therapy of an antibody binding to human CSF-1R for use in the treatment of cancer (including antibodies binding to domains D1-D3 and antibodies binding to domains D4-D5) wherein the CSF-1R antibody is administered in combination with a bispecific ANG-2-VEGF antibody (e.g. an ANG2-VEGF antibody as described in WO2010/040508 or WO2011/117329, in one preferred embodiment with the bispecific ANG-2-VEGF antibody XMab1 as described in WO2011/117329). In one embodiment the antibody binding to human CSF-1R for use in the treatment of cancer is characterized in binding to domains D4-D5. In one embodiment such combination therapy comprises an antibody binding to human CSF-1R, is characterized in that the heavy chain variable domain is SEQ ID NO:39 and the light chain variable domain is SEQ ID NO:40 and the bispecific ANG-2-VEGF antibody XMab1 as described in WO2011/117329.
One further aspect of the invention is the combination therapy of an antibody binding to human CSF-1R (including antibodies binding to domains D1-D3 and antibodies binding to domains D4-D5) with a cancer immunotherapy, wherein the cancer immunotherapy is selected from the group of: cancer vaccines/enhance dendritic cell function: OncoVex (oncolytic virus secreting GM-CSF), an agonistic CD40 antibody, Toll-like receptor (TLR) ligands, TLR agonists, recombinant fusion protein encoding MAGE-A3, PROSTVAC.

One preferred embodiment of the invention is the combination therapy of an antibody binding to human CSF-1R (including antibodies binding to domains D1-D3 and antibodies binding to domains D4-D5, preferably antibodies binding to domains D4-D5 as described herein) with a cancer immunotherapy, wherein the cancer immunotherapy is an agonistic CD40 antibody. CSF-1R antibodies binding to domains D1-D3 of human CSF-1R are described e.g. in WO 2009/026303 and WO 2009/112245 relate to certain anti-CSF-1R antibodies binding to CSF-1R within the first three subdomains (D1 to D3) of the Extracellular Domain (CSF-1R-ECD). WO2011/123381(A1) relates to antibodies against CSF-1R. and Sherr, C. J., et al., Blood 73 (1989) 1786-1793 (typically these antibodies are characterized by inhibiting CSF-1R ligand-dependent but not CSF-1R ligand-independent CSF-1R proliferation and/or signaling).

CSF-1R antibodies binding to domains D4-D5 of human CSF-1R are described e.g. within the present invention, in PCT/EP2012/075241 and Sherr, C. J., et al., Blood 73 (1989) 1786-1793 (typically these antibodies are characterized by inhibiting CSF-1R ligand-dependent and CSF-1R ligand-independent CSF-1R proliferation and/or signaling).

Thus in one aspect of the invention also comprises an antibody binding to human CSF-1R, for use in the treatment of cancer wherein the anti-CSF-1R antibody is administered in combination with a chemotherapeutic agent, radiation, and/or cancer immunotherapy. In one embodiment the cancer immunotherapy is selected the cancer immunotherapy is selected from the group of: a) T cell engaging agents selected from agonistic antibodies, to GITR, to CD27, or to 4-1BB, and T-cell bispecific antibodies (e.g. T cell-engaging BiTE™ antibodies CD3-CD19, CD3-EpCam, CD3-EGFR), IL-2 (Proleukin), Interferon (IFN) alpha, antagonizing antibodies which bind to human CTLA-4 (e.g. ipilimumab), to PD-1, to PD-L1, to TIM-3, to BTLA, to VISTA, to LAG-3, or to CD25, b) targeting immunosuppression: antibodies or small molecules targeting STAT3 or NFkB signaling, blocking IL-1, IL-6, IL-17, IL-23, TNFa function, (e.g antibodies against IL-1, IL-6, IL-17, IL-23, TNFa or against the respective receptor e.g. IL-1R, IL-6R, IL-17R, IL-23R) c) cancer vaccines/enhance dendritic cell function: OncoVex (oncolytic virus secreting GM-CSF), an agonistic CD40 antibody (as described e.g. Beatty et al., Science 331 (2011) 1612-1616, R. H. Vonderheide et al., J Clin Oncol 25, 876 (2007); Khalil, M, et al., Update Cancer Ther. 2007 June 1; 2(2): 61-65, examples in clinical trials are e.g CP-870,893 and dacetuzumab (an agonist CD40 antibody, CAS number 880486-59-9, SGN-40; humanized S2C6 antibody) (Khalil, M, et al, Update Cancer Ther. 2007 June 1; 2(2): 61-65; an agonist CD40 rat anti-mouse IgG2a mAb FGK45 as model antibody is described in S. P. Schoenberger, et al, Nature 393, 480 (1998)) CP-870,893 is a fully human IgG2 CD40 agonist antibody developed by Pfizer. It binds CD40 with a KD of 3.48×10-10 M, but does not block binding of CD40L (see e.g., U.S. Pat. No. 7,338,660 or EP1476185 wherein CP-870,893 is described as antibody 21.4.1). CP-870,893 (antibody 21.4.1 of U.S. Pat. No. 7,338,660) is characterized by comprising (a) a heavy chain variable domain amino acid sequence of QVQLVQSGAEVKKPGASVKVSCKAS GYTFTGYYMHWVRQAPGQGLEWMGWINPDSGGTNYAQKFQGRVTMTR DTSISTAYMELNRLRSDDTAVYYCARDQPLGYCTNGVCSYFDYWGQGTL VTVSS (SEQ ID NO: 88) (which corresponds to SEQ ID NO: 42 of U.S. Pat. No. 7,338,660) (b) a light chain variable domain amino acid sequence of DIQMTQSPSSVSASVGDRVTITCRASQGIYSWLAWYQQKPGKAPNLLIYTA STLQSGVPSRFSGSGSGTDFTLTISSLQPEDFATYYCQQANIFPLTFGGGTKV EIK (SEQ ID NO: 89) ((which corresponds to SEQ ID NO: 44 of U.S. Pat. No. 7,338,660; and/or having the heavy chain variable domain and light chain variable domain amino acid sequences of the antibody produced by hybridoma 21.4.1 having American Type Culture Collection (ATCC) accession number PTA-3605. Dacetuzumab and other humanized S2C6 antibodies are described in U.S. Pat. No. 6,946,129 and U.S. Pat. No. 8,303,955. Humanized S2C6 antibodies are e.g. based on the CDR1, 2 and 3 of the heavy and light chain variable domain of murine mAB S2C6 (deposited with the ATCC as PTA-110). The CDR1, 2 and 3 of the heavy and light chain variable domain of murine mAB S2C6 is described and disclosed U.S. Pat. No. 6,946,129. In one embodiment the agonist CD40 antibody is dacetuzumab. In one embodiment the agonist CD40 antibody is characterized by comprising (a) a heavy chain variable domain amino acid sequence of EVQLVESGGGLVQPGGSLRLSCAASGYSFTGYYIHWVRQAPGKGLEWVA RVIPNAGGTSYNQKFKGRFTLSVDNSKNTAYLQMNSLRAEDTAVYYCARE GIYWWGQGTLVTVS (SEQ ID NO: 90) (b) a light chain variable domain amino acid sequence of DIQMTQSPSSLSASVGDRVTITCRSSQSLVHSNGNTFLHW YQQKPGKAPKLLIYTVSNRFSGVPSRFSGSGSGTDFTLTISSLQPEDFAT YFCSQTTHVPWTFGQGTKVEIKR (SEQ ID NO: 91) Toll-like receptor (TLR) ligands, TLR agonists, recombinant fusion protein encoding MAGE-A3, PROSTVAC; or d) adoptive cell transfer: GVAX (prostate cancer cell line expressing GM-CSF), dendritic cell vaccine, adoptive T cell therapy, adoptive CAR T cell therapy. In one embodiment the cancer immunotherapy is selected from T cell engaging agents selected from IL-2 (Proleukin), and antagonizing antibodies which bind to human CTLA-4 (e.g. ipilimumab). In one embodiment the cancer immunotherapy is IL-2 (Proleukin). In one embodiment the cancer immunotherapy is an antagonizing antibody which bind to human CTLA-4 (e.g. ipilimumab).

In one embodiment cancer immunotherapy, which may be administered with anti-CSF-1R antibody, includes, but is not limited to, targeted therapies. Examples of targeted therapies include, but are not limited to, use of therapeutic antibodies. Exemplary therapeutic antibodies, include, but are not limited to, mouse, mouse-human chimeric, CDR-grafted, humanized and fully human antibodies, and synthetic antibodies, including, but not limited to, those selected by screening antibody libraries. Exemplary antibodies include, but are not limited to, those which bind to cell surface proteins Her2, CDC20, CDC33, mucin-like glycoprotein, and epidermal growth factor receptor (EGFR) present on tumor cells, and optionally induce a cytostatic and/or cytotoxic effect on tumor cells displaying these proteins. Exemplary antibodies also include HERCEPTIN (trastuzumab), which may be used to treat breast cancer and other forms of cancer, and RITUXAN (rituximab), ZEVAL1N (ibritumomab tiuxetan), GLEEVEC (imatinib mesylate), and LYMPHOCIDE (epratuzumab), which may be used to treat non-Hodgkin's lymphoma and other forms of cancer. Certain exemplary antibodies also include ERBITUX (cetuximab) (EMC-C225); ertinolib (Iressa); BEXXAR™ (iodine 131 tositumomab); KDR (kinase domain receptor) inhibitors; anti VEGF antibodies and antagonists (e.g., Avastin (bevacizumab) and VEGAF-TRAP); anti VEGF receptor antibodies and antigen binding regions; anti-Ang-1 and Ang-2 antibodies and antigen binding regions; Ang-2-VEGF bispecific antibodies (as described e.g. in WO2010/040508 or WO2011/117329), antibodies to Tie-2 and other Ang-1 and Ang-2 receptors; Tie-2 ligands; antibodies against Tie-2 kinase inhibitors; inhibitors of Hif-1a, and Campath™ (Alemtuzumab). In certain embodiments, cancer therapy agents are polypeptides which selectively induce apoptosis in tumor cells, including, but not limited to, the TNF-related polypeptide TRAIL. Specific inhibitors of other kinases can also be used in combination with the CSF-1R antibody, including but not limited to, MAPK pathway inhibitors (e.g., inhibitors of ERK, JNK and p38), PBkinase/AKT inhibitors and Pim inhibitors. Other inhibitors include Hsp90 inhibitors, proteasome inhibitors (e.g., Velcade) and multiple mechanism of action inhibitors such as Trisenox.

In one embodiment cancer immunotherapy includes one or more anti-angiogenic agents that decrease angiogenesis. Certain such agents include, but are not limited to, IL-8 antagonists; Campath, B-FGF; FGF antagonists; Tek antagonists (Cerretti et al., U. S. Publication No. 2003/0162712; Cerretti et al., U.S. Pat. No. 6,413,932, and Cerretti et al., U.S. Pat. No. 6,521,424, each of which is incorporated herein by reference for all purposes); anti-TWEAK agents (which include, but are not limited to, antibodies and antigen binding regions); soluble TWEAK receptor antagonists (Wiley, U.S. Pat. No. 6,727,225); an ADAM distintegrin domain to antagonize the binding of integrin to its ligands (Fanslow et al., U. S. Publication No. 2002/0042368); anti-eph receptor and anti-ephrin antibodies; antigen binding regions, or antagonists (U.S. Pat. Nos. 5,981,245; 5,728,813; 5,969,110; 6,596,852; 6,232,447; 6,057,124 and patent family members thereof); anti-VEGF agents (e.g., antibodies or antigen binding regions that specifically bind VEGF, or soluble VEGF receptors or a ligand binding regions thereof) such as Avastin (bevacizumab) or VEGF-TRAP and anti-VEGF receptor agents (e.g., antibodies or antigen binding regions that specifically bind thereto), EGFR inhibitory agents (e.g., antibodies or antigen binding regions that specifically bind thereto) such as panitumumab, IRESSA (gefitinib), TARCEVA (erlotinib), anti-Ang-1 and anti-Ang-2 agents (e.g., antibodies or antigen binding regions specifically binding thereto or to their receptors, e.g., Tie-2/TEK), and anti-Tie-2 kinase inhibitory agents (e.g., antibodies or antigen binding regions that specifically bind and inhibit the activity of growth factors, such as antagonists of hepatocyte growth factor (HGF, also known as Scatter Factor), and antibodies or antigen binding regions that specifically bind its receptor “c-met”; anti-PDGF-BB antagonists; antibodies and antigen binding regions to PDGF-BB ligands; and PDGFR kinase inhibitors. Other anti-angiogenic agents that can be used in combination with an antigen binding protein include agents such as MMP-2 (matrix-metalloproteinase 2) inhibitors, MMP-9 (matrix-metalloproteinase 9) inhibitors, and COX-II (cyclooxygenase II) inhibitors. Examples of useful COX-II inhibitors include CELEBREX (celecoxib), valdecoxib, and rofecoxib. In certain embodiments, cancer therapy agents are angiogenesis inhibitors. Certain such inhibitors include, but are not limited to, SD-7784 (Pfizer, USA); cilengitide. (Merck KGaA, Germany, EP 0 770 622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); semaxanib, (Pfizer, USA, U.S. Pat. No. 5,792,783); vatalanib, (Novartis, Switzerland); 2-methoxyestradiol, (EntreMed, USA); TLC ELL-12, (Elan, Ireland); anecortave acetate, (Alcon, USA); alpha-D148 Mab, (Amgen, USA); CEP-7055, (Cephalon, USA); anti-Vn Mab, (Crucell, Netherlands) DACrantiangiogenic, (ConjuChem, Canada); Angiocidin, (InKine Pharmaceutical, USA); KM-2550, (Kyowa Hakko, Japan); SU-0879, (Pfizer, USA); CGP-79787, (Novartis, Switzerland, EP 0 970 070); ARGENT technology, (Ariad, USA); YIGSR-Stealth, (Johnson & Johnson, USA); fibrinogen-E fragment, (BioActa, UK); angiogenesis inhibitor, (Trigen, UK); TBC-1635, (Encysive Pharmaceuticals, USA); SC-236, (Pfizer, USA); ABT-567, (Abbott, USA); Metastatin, (EntreMed, USA); angiogenesis inhibitor, (Tripep, Sweden); maspin, (Sosei, Japan); 2-methoxyestradiol, (Oncology Sciences Corporation, USA); ER-68203-00, (IVAX, USA); Benefin, (Lane Labs, USA); Tz-93, (Tsumura, Japan); TAN-1120, (Takeda, Japan); FR-111142, (Fujisawa, Japan, JP 02233610); platelet factor 4, (RepliGen, USA, EP 407122); vascular endothelial growth factor antagonist, (Borean, Denmark); cancer therapy, (University of South Carolina, USA); bevacizumab (pINN), (Genentech, USA); angiogenesis inhibitors, (SUGEN, USA); XL 784, (Exelixis, USA); XL 647, (Exelixis, USA); MAb, alpha5beta3 integrin, second generation, (Applied Molecular Evolution, USA and Medlmmune, USA); gene therapy, retinopathy, (Oxford BioMedica, UK); enzastaurin hydrochloride (USAN), (Lilly, USA); CEP 7055, (Cephalon, USA and Sanofi-Synthelabo, France); BC 1, (Genoa Institute of Cancer Research, Italy); angiogenesis inhibitor, (Alchemia, Australia); VEGF antagonist, (Regeneron, USA); rBPI 21 and BPI-derived antiangiogenic, (XOMA, USA); PI 88, (Progen, Australia); cilengitide (pINN), (Merck KGaA, German; Munich Technical University, Germany, Scripps Clinic and Research Foundation, USA); cetuximab (INN), (Aventis, France); AVE 8062, (Ajinomoto, Japan); AS 1404, (Cancer Research Laboratory, New Zealand); SG 292, (Telios, USA); Endostatin, (Boston Childrens Hospital, USA); ATN 161, (Attenuon, USA); ANGIOSTATIN, (Boston Childrens Hospital, USA); 2-methoxyestradiol, (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProIX, USA); METASTATIN, (EntreMed, USA); troponin 1, (Harvard University, USA); SU 6668, (SUGEN, USA); OXI 4503 (OXiGENE, USA); o-guanidines, (Dimensional Pharmaceuticals, USA); motuporamine C, (British Columbia University, Canada); CDP 791, (Celltech Group, UK); atiprimod (PINN), (GlaxoSmithKline, UK); E 7820, (Eisai, Japan); CYC 381, (Harvard University, USA); AE 941, (Aeterna, Canada); vaccine, angiogenesis, (EntreMed, USA); urokinase plasminogen activator inhibitor, (Dendreon, USA); oglufanide (pINN), (Melmotte, USA); HIF-I alfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol, (EntreMed, USA); anginex, (Maastricht University, Netherlands, and Minnesota University, USA); ABT 510, (Abbott, USA); ML 993, (Novartis, Switzerland); VEGI, (Proteom Tech, USA); tumor necrosis factor-alpha inhibitors, (National Institute on Aging, USA); SU 11248, (Pfizer, USA and SUGEN USA); ABT 518, (Abbott, USA); YH1 6, (Yantai Rongchang, China); S-3APG, (Boston Childrens Hospital, USA and EntreMed, USA); MAb, KDR, (ImClone Systems, USA); MAb, alpha5 betal, (Protein Design, USA); KDR kinase inhibitor, (Celltech Group, UK, and Johnson & Johnson, USA); GFB 116, (South Florida University, USA and Yale University, USA); CS 706, (Sankyo, Japan); combretastatin A4 prodrug, (Arizona State University, USA); chondroitinase AC, (IBEX, Canada); BAY RES 2690, (Bayer, Germany); AGM 1470, (Harvard University, USA, Takeda, Japan, and TAP, USA); AG 13925, (Agouron, USA); Tetrathiomolybdate, (University of Michigan, USA); GCS 100, (Wayne State University, USA) CV 247, (Ivy Medical, UK); CKD 732, (Chong Kun Dang, South Korea); MAb, vascular endothelium growth factor, (Xenova, UK); irsogladine (INN), (Nippon Shinyaku, Japan); RG 13577, (Aventis, France); WX 360, (Wilex, Germany); squalamine (pINN), (Genaera, USA); RPI 4610, (Sima, USA); cancer therapy, (Marinova, Australia); heparanase inhibitors, (InSight, Israel); KL 3106, (Kolon, South Korea); Honokiol, (Emory University, USA); ZK CDK, (Schering AG, Germany); ZK Angio, (Schering AG, Germany); ZK 229561, (Novartis, Switzerland, and Schering AG, Germany); XMP 300, (XOMA, USA); VGA 1102, (Taisho, Japan); VEGF receptor modulators, (Pharmacopeia, USA); VE-cadherin-2 antagonists, (ImClone Systems, USA); Vasostatin, (National Institutes of Health, USA); vaccine, FIk-I, (ImClone Systems, USA); TZ 93, (Tsumura, Japan); TumStatin, (Beth Israel Hospital, USA); truncated soluble FLT 1 (vascular endothelial growth factor receptor 1), (Merck & Co, USA); Tie-2 ligands, (Regeneron, USA); thrombospondin 1 inhibitor, (Allegheny Health, Education and Research Foundation, USA); 2-Benzenesulfonamide, 4-(5-(4-chlorophenyl)-3-(trifluoromethyl)-1H-pyrazol-1-yl)-; Arriva; and C-MeL AVE 8062 ((2S)-2-amino-3-hydroxy-N-[2-methoxy-5-[(1Z)-2-(3,4,5-tri-methoxyphenyl)ethenyl]phenyl]propanamide monohydrochloride); metelimumab (pINN)(immunoglobulin G4, anti-(human transforming growth factor.beta.1 (human monoclonal CAT 192.gamma.4-chain)), disulfide with human monoclonal CAT 192.kappa.-chain dimer); Flt3 ligand; CD40 ligand; interleukin-2; interleukin-12; 4-1BB ligand; anti-4-IBB antibodies; TNF antagonists and TNF receptor antagonists including TNFR/Fc, TWEAK antagonists and TWEAK-R antagonists including TWEAK-R/Fc; TRAIL; VEGF antagonists including anti-VEGF antibodies; VEGF receptor (including VEGF-R1 and VEGF-R2, also known as Fltl and Flkl or KDR) antagonists; CD1 48 (also referred to as DEP-I, ECRTP, and PTPRJ, see Takahashi et al., J. Am. Soc. Nephrol. 10 (1999) 2135-1245, hereby incorporated by reference for any purpose) agonists; thrombospondin 1 inhibitor, and inhibitors of one or both of Tie-2 or Tie-2 ligands (such as Ang-2). A number of inhibitors of Ang-2 are known in the art, including anti-Ang-2 antibodies described in published U. S. Patent Application No. 2003/0124129 (corresponding to PCT Application No. WO 2003/030833), and U.S. Pat. No. 6,166,185, the contents of which are hereby incorporated by reference in their entirety. Additionally, Ang-2 peptibodies are also known in the art, and can be found in, for example, published U. S. Patent Application No. 2003/0229023 (corresponding to PCT Application No. WO 2003/057134), and published U. S. Patent Application No. 2003/0236193, the contents of which are hereby incorporated by reference in their entirety for all purposes. Certain chemotherapeutic therapy agents include, but are not limited to: thalidomide and thalidomide analogues (N-(2,6-dioxo-3-piperidyl)phthalimide); tecogalan sodium (sulfated polysaccharide peptidoglycan); TAN 1120 (S-acetyl-V-1-O-tetrahydro-11-trihydroxy-1-methoxy-10-[[octahydro-5-hydroxy-2-(2-hydroxypropyl)-4,10-dimethyl.rho.yrano[3,4-d]-1,3,6-dioxazocin-8-yl]oxy]-5,12-naphthacenedione); suradista (7,7′-[carbonylbis[imino(1-met-hyl-1H-pyrrole-4,2-diyl)carbonylimino(1-methyl-1H-pyrrole-4,2-diyl)carbony-limino]]bis-1,3-naphthalenedisulfonic acid tetrasodium salt); SU 302; SU 301; SU 1498 ((E)-2-cyano-3-[4-hydroxy-3,5-bis(1-methylethyl)phenyl]-N-(3-phenylpropyl)-2-propenamide); SU 1433 (4-(6,7-dimethyl-2-quinoxalinyl)-1-,2-benzenediol); ST 1514; SR 25989; soluble Tie-2; SERM derivatives, Pharmos; semaxanib (pINN)(3-[(3,5-dimethyl-1H-pyrrol-2-yl)methylene]-1,3″ dihydro-2H-indol-2-one); S 836; RG 8803; RESTIN; R 440 (3-(1-methyl-1H-indol-3-yl)-4-(1-methyl-6-nitro-1H-indol-3-yl)-1H-pyrrole-2,5-dione); R 123942 (1-[6-(1,2,4-thiadiazol-5-yl)-3-pyridazinyl]-N-[3-(t-rifluoromethyl)phenyl]-4-rsho.iperidinamine); prolyl hydroxylase inhibitor; progression elevated genes; prinomastat (INN) ((S)-2,2-dimethyl-4-[[p-(4-pyridyloxy)phenyl]sulphonyl]-3-thiomorpholinecarbohydroxamic acid); NV 1030; NM 3 (8-hydroxy-6-methoxy-alpha-methyl-1-oxo-1H-2-benzopyran-3-acet-ic acid); NF 681; NF 050; MIG; METH 2; METH 1; manassantin B (alpha-[1-[4-[5-[4-[2-(3,4-dimethoxyphenyl)-2-hydroxy-1-methylethoxy]-3-methoxyphenyl]tetrahydro-3,4-dimethyl-2-furanyl]-2-methoxyphenoxy]ethyl]-1,-3-benzodioxole-5-methanol); KDR monoclonal antibody; alpha5beta3 integrin monoclonal antibody; LY 290293 (2-amino-4-(3-pyridinyl)-4H-naphtho[1,2-b]-pyran-3-carbon itrile); KP 0201448; KM 2550; integrin-specific peptides; INGN 401; GYKI 66475; GYM 66462; greenstatin (101-354-plasminogen (human)); gene therapy for rheumatoid arthritis, prostate cancer, ovarian cancer, glioma, endostatin, colorectal cancer, ATF BTPI, antiangiogenesis genes, angiogenesis inhibitor, or angiogenesis; gelatinase inhibitor, FR 111142 (4,5-dihydroxy-2-hexenoic acid 5-methoxy-4-[2-methyl-3-(3-methyl-2-butenyl)oxiranyl]-1-oxaspiro[2.5]oct-6-yl ester); forfenimex (PINN) (S)-alpha-amino-3-hydroxy-4-(hydroxymethyl)benzeneacetic acid); fibronectin antagonist (1-acetyl-L-prolyl-L-histidyl-L-seryl-L-cysteinyl-L-aspartamide); fibroblast growth factor receptor inhibitor; fibroblast growth factor antagonist; FCE 27164 (7,7′-[carbonylbis[imino(1-methyl-1H-pyrrole-4,2-diyl)carbonylimino(1-methyl-1H-pyrrole-4,2-diyl)carbonylimino]-]bis-1,3,5-naphthalenetrisulfonic acid hexasodium salt); FCE26752 (8,8′-[carbonylbis[imino(1-methyl-1H-pyrrole-4,2-diyl)carbonylimino(1-met-hyl-1H-pyrrole-4,2-diyl)carbonylimino]]bis-1,3,6-naphthalenetrisulfonic acid); endothelial monocyte activating polypeptide II; VEGFR antisense oligonucleotide; anti-angiogenic and trophic factors; ANCHOR angiostatic agent; endostatin; DeI-I angiogenic protein; CT 3577; contortrostatin; CM 101; chondroitinase AC; CDP 845; CanStatin; BST 2002; BST 2001; BLS 0597; BIBF 1000; ARRESTIN; apomigren (1304-1388-type XV collagen (human gene COL15A1 alpha1-chain precursor)); angioinhibin; aaATIII; A 36; 9alpha-fluoromedroxyprogesterone acetate ((6-alpha)-17-(acetyloxy)-9-fluo-ro-6-methyl-pregn-4-ene-3,20-dione); 2-methyl-2-phthalimidino-glutaric acid (2-(1,3-dihydro-1-oxo-2H-isoindol-2-yl)-2-methylpentanedioic acid); Yttrium 90 labelled monoclonal antibody BC-I; Semaxanib (3-(4,5-Dimethylpyrrol-2-ylmethylene)indolin-2-one)(C15 H14 N2 O); PI 88 (phosphomannopentaose sulfate); Alvocidib (4H-1-Benzopyran-4-one, 2-(2-chlorophenyl)-5,7-dihydroxy-8-(3-hydroxy-1-methyl-4-piperidinyl)-cis-(−)-) (C21-H20 Cl N 05); E 7820; SU 11248 (5-[3-Fluoro-2-oxo-1,2-dihydroi-ndol-(3Z)-ylidenemethyl]-2,4-dimethyl-1H-pyrrole-3-carboxylic acid (2-diethylaminoethyl)amide) (C22 H27 F N4 O2); Squalamine (Cholestane-7,24-diol, 3-[[3-[(4-aminobutyl)aminopropyl]amino]-, 24-(hydrogen sulfate), (3.beta.,5.alpha.,7.alpha.)-) (C34 H65 N3 O.sub.5 S); Eriochrome Black T; AGM 1470 (Carbamic acid, (chloroacetyl)-, 5-methoxy-4-[2-methyl-3-(3-methyl-2-butenyl)oxiranyl]-1-oxaspiro[2,5]oct-6-yl ester, [3R-[3alpha, 4alpha(2R, 3R), 5beta, 6beta]]) (C 19 H28 Cl N 06); AZD 9935; BIBF 1000; AZD 2171; ABT 828; KS-interleukin-2; Uteroglobin; A 6; NSC 639366 (1-[3-(Diethylamino)-2-hydroxypropylamino]-4-(oxyran-2-ylmethylamino)anthra-quinone fumerate) (C24 H29 N3 O4. C4 H4 04); ISV 616; anti-ED-B fusion proteins; HUI 77; Troponin I; BC-I monoclonal antibody; SPV 5.2; ER 68203; CKD 731 (3-(3,4,5-Trimethoxypheny-1)-2(E)-sho.ropenoic acid (3R,4S,5S,6R)-4-[2(R)-methyl-3(R)-3(R)-(3-methyl-2-butenyl)oxiran-2-yl]-5-methoxy-1-oxaspiro-[2.5]oct-6-yl ester) (C28 H38 O8); IMC-ICl 1; aaATIII; SC 7; CM 101; Angiocol; Kringle 5; CKD 732 (3-[4-[2-(Dimethylamino)ethoxy]phenyl]-2(E)-propenoic acid)(C29 H41 N 06); U 995; Canstatin; SQ 885; CT 2584 (1-[11-(Dodecylamino)-10-hydroxyun-decyl]-3,7-dimethylxanthine)(C30H55N5 O3); Salmosin; EMAP II; TX 1920 (1-(4-Methylpiperazino)-2-(2-nitro-1H-1-imidazoyl)-1-ethanone) (ClO Hl 5 N5 03); Alpha-v Beta-x inhibitor; CHER. 11509 (N-(I-Propynyl)glycyl-[N-(2-naphthyl)]glycyl-[N-(carbamoylmethyl)]glycinebis(4-methoxyphenyl) methyl-amide)(C36 H37 N5 O6); BST 2002; BST 2001; B 0829; FR 111142; 4,5-Dihydroxy-2(E)-hexenoic acid (3R,4S,5S,6R)-4-[1(R),2(R)-epoxy-1,5-dim-ethyl-4-hexenyl]-5-methoxy-1-oxaspiro[2.5]octan-6-yl ester (C22 H34 O7); and kinase inhibitors including, but not limited to, N-(4-chlorophenyl)-4-(4-pyridinylmethyl)-1-phthalazinamine; 4-[4-[[[[4-chloro-3-(trifluoromethyl)phenyl]amino]carbonyl]amino]phenoxy]-N-methyl-2-pyridinecarboxamide; N-[2-(diethylamino)ethyl]-5-[(5-fluoro-1,-2-dihydro-2-oxo-3H-indol-3-ylidene)methyl]-2,4-dimethyl-1H-pyrrole-3-carbo-xamide; 3-[(4-bromo-2,6-difluorophenyl)methoxy]-5-[[[[4-(1-pyrrolidinyl)bu-tyl]amino]carbonyl]amino]-4-isothiazolecarboxamide; N-(4-bromo-2-fluorophenyl)-6-methoxy-7-[(1-methyl-4-piperidinyl)methoxy]-4-quinazolinamine; 3-[5,6,7, 13-tetrahydro-9-[(1-methylethoxy)methyl]-5-oxo-12H-indeno[2, 1-a]pyrrolo[3,4-c]carbazol-1 2-yl]propyl ester N,N-dimethyl-glycine; N-[5-[[[5-(1, 1-dimethylethyl)-2-oxazolyl]methyl]thi-o]-2-thiazolyl]-4-piperidinecarboxamide; N-[3-chloro-4-[(3-fluorophenyl)me-thoxy]phenyl]-6-[5-[[[2-(methylsulfonyl)ethyl]amino]methyl]-2-furanyl]4-quinazolinamine; 4-[(4-Methyl-1-piperazinyl)methyl]-N-[4-methyl-3-[[4-(3-pyridinyl)-2-pyrimidinyl]amino]-phenyl]benzamide; N43-chloro-4-fluorophenyl)-7-methoxy-6-[3-(4-morpholinyl)propoxy]-4-quinazolinamine; N-(3-ethynylphenyl)-6,7-bis(2-methoxyethoxy)-4-quinazolinamine; N-3-((((2R)-1-methyl-2-pyrrolidinyl)methyl)-oxy)-5-(trifluoromethyl)pheny-1)-2-((3-(1,3-oxazol-5-yl)phenyl)amino)-3-pyridinecarboxamide; 2-(((4-fluorophenyl)methyl)amino)-N-(3-((((2R)-1-methyl-2-pyrrolidinyl)me-thyl)oxy)-5-(trifluoromethyl)phenyl)-3-pyridinecarboxamide; N-[3-(Azetidin-3-ylmethoxy)-5-trifluoromethyl-phenyl]-2-(4-fluoro-benzyla-mino)-nicotinamide; 6-fluoro-N-(4-(1-methylethyl)phenyl)-2-((4-pyridinylme-thyl)amino)-3-pyridinecarboxamide; 2-((4-pyridinylmethyl)amino)-N-(3-(((2S-)-2-pyrrolidinylmethyl)oxy)-5-(trifluoromethyl)phenyl)-3 pyridinecarboxami-de; N-(3-(1,1-dimethylethyl)-1H-pyrazol-5-yl)-2-((4-pyridinylmethyl)amino)-3-pyridinecarboxamide; N-(3,3-dimethyl-2,3-dihydro-1-benzofuran-6-yl)-2-(-(4-pyridinylmethyl)amino)-3-pyridinecarboxamide; N-(3-((((2S)-1-methyl-2-p-yrrolidinyemethyl)oxy)-5-(trifluoromethyl)phenyl)-2-((4-pyridinylmethyl)amino)-3-pyridinecarboxamide; 2-((4-pyridinylmethyl)amino)-N-(3-((2-(1-pyrr-olidinyl)ethyl)oxy)-4-(trifluoromethyl)phenyl)-3-pyridinecarboxamide; N-(3,3-dimethyl-2,3-dihydro-1H-indol-6-yl)-2-((4-pyridinylmethyl)amino)-3-pyridinecarboxamide; N-(4-(pentafluoroethyl)-3-(((2S)-2-pyrrolidinylmethyl)oxy)phenyl)-2-((4-pyridinylmethyl)amino)-3-pyridinecarboxamide; N-(3-((3-azetidinylmethyl)oxy)-5-(trifluoromethyl)phenyl)-2-((4-pyridinyl-methyl)amino)-3-pyridinecarboxamide; N-(3-(4-piperidinyloxy)-5-(trifluorom-ethyl)phenyl)-2-((2-(3-pyridinyl)ethyl)amino)-3-pyridinecarboxamide; N-(4,4-dimethyl-1,2,3,4-tetrahydroisoquinolin-7-yl)-2-(1H-indazol-6-ylami-no)-nicotinamide; 2-(1H-indazol-6-ylamino)-N-[3-(1-methylpyrrolidin-2-ylme-thoxy)-5-trifluoromethyl-phenyl]-nicotinamide; N-[1-(2-dimethylamino-acety-1)-3,3-dimethyl-2,3-dihydro-1H-indol-6-yl]-2-(1H-indazol-6-ylamino)-nicoti-namide; 2-(1H-indazol-6-ylamino)-N-[3-(pyrrolidin-2-ylmethoxy)-5-trifluoro-methyl-phenyl]-nicotinamide; N-(I-acetyl-S-dimethylS-dihydro-1H-indol-6-yl)-2-(1H-indazol-6-ylamino)-nicotinamide; N-(4,4-dimethyl-1-oxo-1,2,3,-4-tetrahydro-isoquinolin-7-yl)-2-(1H-indazol-6-ylamino)-nicotinamide; N-[4-(tert-butyl)-3-(3-piperidylpropyl)phenyl][2-(1H-indazol-6-ylamino)(3-pyridyl)]carboxamide; N-[5-(tert-butyl)isoxazol-3-yl][2-(1H-indazol-6-yla-mino)(3-pyridyl)]carboxamide; and N-[4-(tert-butyl)phenyl][2-(1H-indazol-6-ylamino)(3-pyridyl)]carboxamide, and kinase inhibitors disclosed in U.S. Pat. Nos. 6,258,812; 6,235,764; 6,630,500; 6,515,004; 6,713,485; 5,521,184; 5,770,599; 5,747,498; 5,990,141; U. S. Publication No. U.S. 2003/0105091; and Patent Cooperation Treaty publication nos. WO 01/37820; WO 01/32651; WO 02/68406; WO 02/66470; WO 02/55501; WO 04/05279; WO 04/07481; WO 04/07458; WO 04/09784; WO 02/59110; WO 99/45009; WO 98/35958; WO 00/59509; WO 99/61422; WO 00/12089; and WO 00/02871, each of which publications are hereby incorporated by reference for all purposes.

In one embodiment cancer immunotherapy, which may be administered with anti-CSF-1R antibody, includes, but is not limited to, a growth factor inhibitor. Examples of such agents, include, but are not limited to, agents that can inhibit EGF-R (epidermal growth factor receptor) responses, such as EGF-R antibodies, EGF antibodies, and molecules that are EGF-R inhibitors; VEGF (vascular endothelial growth factor) inhibitors, such as VEGF receptors and molecules that can inhibit VEGF; and erbB2 receptor inhibitors, such as organic molecules or antibodies that bind to the erbB2 receptor, for example, HERCEPTIN (trastuzumab) (Genentech, Inc.). EGF-R inhibitors are described in, for example in U.S. Pat. No. 5,747,498, WO 98/14451, WO 95/19970, and WO 98/02434. In one embodiment of the invention radiation may be carried out and/or a radiopharmaceutical may be used in addition to the anti-CSF-1R antibody. The source of radiation can be either external or internal to the patient being treated. When the source is external to the patient, the therapy is known as external beam radiation therapy (EBRT). When the source of radiation is internal to the patient, the treatment is called brachytherapy (BT). Radioactive atoms for use in the context of this invention can be selected from, e.g., radium, cesium-137, iridium-192, americium-241, gold-198, cobalt-57, copper-67, technetium-99, iodine-123, iodine-131, and indium-111. Is also possible to label the antibody with such radioactive isotopes.

Radiation therapy is a standard treatment for controlling unresectable or inoperable tumors and/or tumor metastases. Improved results have been seen when radiation therapy has been combined with chemotherapy. Radiation therapy is based on the principle that high-dose radiation delivered to a target area will result in the death of reproductive cells in both tumor and normal tissues. The radiation dosage regimen is generally defined in terms of radiation absorbed dose (Gy), time and fractionation, and must be carefully defined by the oncologist. The amount of radiation a patient receives will depend on various considerations, but the two most important are the location of the tumor in relation to other critical structures or organs of the body, and the extent to which the tumor has spread. A typical course of treatment for a patient undergoing radiation therapy will be a treatment schedule over a 1 to 6 week period, with a total dose of between 10 and 80 Gy administered to the patient in a single daily fraction of about 1.8 to 2.0 Gy, 5 days a week. In a preferred embodiment of this invention there is synergy when tumors in human patients are treated with the combination treatment of the invention and radiation. In other words, the inhibition of tumor growth by means of the agents comprising the combination of the invention is enhanced when combined with radiation, optionally with additional chemotherapeutic or anticancer agents. Parameters of adjuvant radiation therapies are, for example, contained in WO 99/60023. In one embodiment of the invention the anti-CSF-1R antibody is characterized in that the antibody binds to human CSF-1R fragment delD4 (SEQ ID NO: 65) and to human CSF-1R Extracellular Domain (SEQ ID NO: 64) with a ratio of 1:50 or lower.

In one embodiment of the invention the antibody is characterized in that the antibody does not bind to human CSF-1R fragment delD4 (SEQ ID NO: 65).

In one embodiment of the invention the antibody is characterized in that
a) the heavy chain variable domain is SEQ ID NO:7 and the light chain variable domain is SEQ ID NO:8,
b) the heavy chain variable domain is SEQ ID NO:15 and the light chain variable domain is SEQ ID NO:16;
c) the heavy chain variable domain is SEQ ID NO:75 and the light chain variable domain is SEQ ID NO:76;
d) the heavy chain variable domain is SEQ ID NO:83 and the light chain variable domain is SEQ ID NO:84;
   or a humanized version thereof.

In one embodiment of the invention the antibody is characterized in that
a) the heavy chain variable domain is SEQ ID NO:7 and the light chain variable domain is SEQ ID NO:8,
b) the heavy chain variable domain is SEQ ID NO:15 and the light chain variable domain is SEQ ID NO:16;
   or a humanized version thereof.

In one embodiment of the invention the antibody is characterized in that
a) the heavy chain variable domain is SEQ ID NO:23 and the light chain variable domain is SEQ ID NO:24, or
b) the heavy chain variable domain is SEQ ID NO:31 and the light chain variable domain is SEQ ID NO:32, or
c) the heavy chain variable domain is SEQ ID NO:39 and the light chain variable domain is SEQ ID NO:40, or
d) the heavy chain variable domain is SEQ ID NO:47 and the light chain variable domain is SEQ ID NO:48, or
e) the heavy chain variable domain is SEQ ID NO:55 and the light chain variable domain is SEQ ID NO:56.

In one embodiment of the invention the antibody is characterized in that
a) the heavy chain variable domain comprises a CDR3 region of SEQ ID NO: 1, a CDR2 region of SEQ ID NO: 2, and a CDR1 region of SEQ ID NO:3, and the light chain variable domain comprises a CDR3 region of SEQ ID NO: 4, a CDR2 region of SEQ ID NO:5, and a CDR1 region of SEQ ID NO:6, or
b) the heavy chain variable domain comprises a CDR3 region of SEQ ID NO: 9, a CDR2 region of SEQ ID NO: 10, and a CDR1 region of SEQ ID NO: 11, and the light chain variable domain comprises a CDR3 region of SEQ ID NO:12, a CDR2 region of SEQ ID NO: 13, and a CDR1 region of SEQ ID NO: 14, or
c) the heavy chain variable domain comprises a CDR3 region of SEQ ID NO: 17, a CDR2 region of SEQ ID NO: 18, and a CDR1 region of SEQ ID NO:19, and the light chain variable domain comprises a CDR3 region of SEQ ID NO: 20, a CDR2 region of SEQ ID NO:21, and a CDR1 region of SEQ ID NO:22, or
d) the heavy chain variable domain comprises a CDR3 region of SEQ ID NO: 25, a CDR2 region of SEQ ID NO: 26, and a CDR1 region of SEQ ID NO: 27, and the light chain variable domain comprises a CDR3 region of SEQ ID NO:28, a CDR2 region of SEQ ID NO: 29, and a CDR1 region of SEQ ID NO: 30, or
e) the heavy chain variable domain comprises a CDR3 region of SEQ ID NO: 33, a CDR2 region of SEQ ID NO: 34, and a CDR1 region of SEQ ID NO: 35, and the light chain variable domain comprises a CDR3 region of SEQ ID NO:36, a CDR2 region of SEQ ID NO: 37, and a CDR1 region of SEQ ID NO: 38, or
f) the heavy chain variable domain comprises a CDR3 region of SEQ ID NO:41, a CDR2 region of SEQ ID NO: 42, and a CDR1 region of SEQ ID NO:43, and the light chain variable domain comprises a CDR3 region of SEQ ID NO: 44, a CDR2 region of SEQ ID NO:45, and a CDR1 region of SEQ ID NO:46, or
g) the heavy chain variable domain comprises a CDR3 region of SEQ ID NO: 49, a CDR2 region of SEQ ID NO: 50, and a CDR1 region of SEQ ID NO: 51, and the light chain variable domain comprises a CDR3 region of SEQ ID NO:52, a CDR2 region of SEQ ID NO: 53, and a CDR1 region of SEQ ID NO: 54; or
h) the heavy chain variable domain comprises a CDR3 region of SEQ ID NO:69, a CDR2 region of SEQ ID NO: 70, and a CDR1 region of SEQ ID NO:71, and the light chain variable domain comprises a CDR3 region of SEQ ID NO: 72, a CDR2 region of SEQ ID NO:73, and a CDR1 region of SEQ ID NO:74, or
i) the heavy chain variable domain comprises a CDR3 region of SEQ ID NO: 77, a CDR2 region of SEQ ID NO: 78, and a CDR1 region of SEQ ID NO: 79, and the light chain variable domain comprises a CDR3 region of SEQ ID NO:80, a CDR2 region of SEQ ID NO: 81, and a CDR1 region of SEQ ID NO: 82.

In one embodiment of the invention the antibody is of human IgG1 subclass or of human IgG4 subclass.

A further embodiment of the invention is a pharmaceutical composition comprising an antibody according to the invention.

The invention further comprises the use an of an antibody according to the invention for the manufacture of a medicament for treatment of a CSF-1R mediated disease.

The invention further comprises the use an of an antibody according to the invention for the manufacture of a medicament for treatment of cancer.

The invention further comprises the use an of an antibody according to the invention for the manufacture of a medicament for treatment of bone loss.

The invention further comprises the use an of an antibody according to the invention for the manufacture of a medicament for treatment of metastasis.

The invention further comprises the use an of an antibody according to the invention for the manufacture of a medicament for treatment of inflammatory diseases.

The invention further comprises an antibody according to the invention for treatment of a CSF-1R mediated disease.

The invention further comprises an antibody according to the invention for treatment of cancer.

The invention further comprises an antibody according to the invention for treatment of bone loss.

The invention further comprises an antibody according to the invention for treatment of metastasis.

The invention further comprises an antibody according to the invention for treatment of inflammatory diseases.

The combination therapies of the antibodies described herein show benefits for patients in need of a CSF-1R targeting therapy. The antibodies according to the invention show efficient antiproliferative activity against ligand-independent and ligand-dependent proliferation and are therefore especially useful in the treatment of cancer and metastasis in combination with a chemotherapeutic agent, radiation and/or cancer immunotherapy.

The invention further provides a method for treating a patient suffering from cancer, comprising administering to a patient diagnosed as having such a disease (and therefore being in need of such a therapy) an effective amount of an antibody according to the invention in combination with a chemotherapeutic agent, radiation and/or cancer immunotherapy. The antibody is administered preferably in a pharmaceutical composition.

Surprisingly it has been found that, using a human CSF-1R fragment delD4 in which the D4 subdomain of human CSF-1R-ECD was deleted (SEQ ID NO:65), the anti-CSF-1R antibodies could be selected. These antibodies show valuable properties like excellent ligand-dependent cell growth inhibition and at the same time ligand independent cell growth inhibition of NIH 3T3 cell, retrovirally infected with either an expression vector for full-length wildtype CSF-1R (SEQ ID NO:62) or mutant CSF-1R L301S Y969F (SEQ ID NO:63) whereby mutant CSF-1R recombinant cells are able to form spheroids independent of the CSF-1 ligand. Furthermore these antibodies inhibit (both) human and cynomolgous macrophage differentiation, as they inhibit survival of human and cynomolgous monocytes. Further antibodies binding to the binding to the (dimerization) domains D4 to D5 can be selected by screening for antibodies that bind to the complete extracellular domain of human CSF-1R (SEQ ID NO: 64) (including domains D1 to D5), and not binding to the domains D1 to D3 (SEQ ID NO: 66) of the extracellular domain of human CSF-1R.

### DESCRIPTION OF THE DRAWINGS

FIGS. 1A and 1B depict data demonstrating inhibition of BeWo tumor cells in 3D culture under treatment with different anti-CSF-1R monoclonal antibodies at a concentration of 10 μg/ml. Y axis: viability normalized mean relative light units (RLU) corresponding to the ATP-content of the cells (CellTiterGlo assay).

X axis: tested probes: Minimal Medium (0.5% FBS), mouse IgG1 (mIgG1, 10 μg/ml), mouse IgG2a (mIgG2a 10 μg/ml), CSF-1 only, Mab 2F11, Mab 2E10, Mab2H7, Mab1G10 and SC 2-4A5. Highest inhibition of CSF-1 induced growth was observed with the anti-CSF-1R antibodies according to the invention.

FIG. 2A depicts a Biacore sensogram of binding of different anti-CSF-1R antibodies to immobilized human CSF-1R fragment delD4 (comprising the extracellular subdomains D1-D3 and D5) (SEQ ID NO: 65) (y-axis: binding signal in Response Units (RU), baseline=0 RU, x-axis: time in seconds (s)): While the antibodies Mab 3291 and sc 2-4A5 clearly show binding to this delD4 fragment, the antibodies according to the invention e.g. Mab 2F11, and Mab 2E10, did not bind to the CSF-1R fragment delD4. The control anti-CCR5 antibody m<CCR5>Pz03.1C5 did also not bind to the CSF-1R fragment delD4.

FIG. 2B depicts a Biacore sensogram of binding of different anti-CSF-1R antibodies to immobilized human CSF-1R Extracellular Domain (CSF-1R-ECD) (comprising the extracellular subdomains D1-D5) (SEQ ID NO: 64) (y-axis: binding signal in Response Units (RU), baseline=0 RU, x-axis: time in seconds (s)):

All anti-CSF-1R antibodies show binding to CSF-1R-ECD. The control anti-CCR5 antibody m<CCR5>Pz03.1C5 did not bind to the CSF-1R-ECD.

FIG. 2C depicts a Biacore sensogram of binding of different anti-CSF-1R antibodies to immobilized human CSF-1R fragment delD4 (comprising the extracellular subdomains D1-D3 and D5) (SEQ ID NO: 65) (y-axis: binding signal in Response Units (RU), baseline=0 RU, x-axis: time in seconds (s)): Mab 1G10, Mab 2H7 and humanized hMab 2F11-e7 did not bind to the CSF-1R fragment delD4. The control anti-CCR5 antibody m<CCR5>Pz03.1C5 did also not bind to the CSF-1R fragment delD4.

FIG. 2D depicts a Biacore sensogram of binding of different anti-CSF-1R antibodies to immobilized human CSF-1R Extracellular Domain (CSF-1R-ECD) (comprising the extracellular subdomains D1-D5) (SEQ ID NO: 64) (y-axis: binding signal in Response Units (RU), baseline=0 RU, x-axis: time in seconds (s)): All anti-CSF-1R antibodies Mab 1G10, Mab 2H7 and humanized hMab 2F11-e7 showed binding to CSF-1R-ECD. The control anti-CCR5 antibody m<CCR5>Pz03.1C5 did not bind to the CSF-1R-ECD.

FIG. 2E depicts a Biacore sensogram of binding of different anti-CSF-1R antibodies to immobilized human CSF-1R fragment delD4 (comprising the extracellular subdomains D1-D3 and D5) (SEQ ID NO: 65) (y-axis: binding signal in Response Units (RU), baseline=0 RU, x-axis: time in seconds (s)): All anti-CSF-1R antibodies 1.2.SM, CXIIG6, ab10676 and MAB3291 show binding to the CSF-1R fragment delD4. The control anti-CCR5 antibody m<CCR5>Pz03.1C5 did also not bind to the CSF-1R fragment delD4.

FIG. 2F depicts a Biacore sensogram of binding of different anti-CSF-1R antibodies to immobilized human CSF-1R Extracellular Domain (CSF-1R-ECD) (comprising the extracellular subdomains D1-D5) (SEQ ID NO: 64) (y-axis: binding signal in Response Units (RU), baseline=0 RU, x-axis: time in seconds (s)):

All anti-CSF-1R antibodies 1.2.SM, CXIIG6, ab10676 and MAB3291 show binding to CSF-1R-ECD. The control anti-CCR5 antibody m<CCR5>Pz03.1C5 did not bind to the CSF-1R-ECD.

FIGS. 3A, 3B, 3C, and 3D depict data showing CSF-1 levels in Cynomolgous monkey after application of different dosages of anti-CSF-1R antibody according to the invention.

FIG. 4 depicts data demonstrating the in vivo efficacy—tumor growth inhibition of anti-CSF-1R antibodies according to the invention in breast cancer BT20 xenograft.

FIG. 5A depicts data demonstrating Human Monocytes differentiated into macrophages with coculture of GM-CSF or CSF-1 (100 ng/ml ligand). After 6 days differentiation addition of RO7155. Cell viability was measured at day 7 of antibody treatment in a CTG Viability Assay (CellTiterGlo® Promega). Calculation of % cell viability: RLU signals from treated cells divided by RLU signal from untreated control without antibody, (n=4).

FIG. 5B depicts data demonstrating: Human Monocytes differentiated into macrophages with GM-CSF (M1) or M-CSF (M2) for 7 days. Phenotype analyzed by indirect fluorescence analysis-staining with anti CD163-PE, anti CD80-PE or anti HLA-DR/DQ/DP-Zenon-Alexa647 labeled. The number in each histogram corresponds to mean ratio fluorescence intensity (MRFI); calculated ratio between mean fluorescence intensity (MFI) of cells stained with the selected antibody (empty histogram) and of corresponding isotype control (negative control; gray filled histogram) (mean±SD; n≧5).

FIGS. 6A, 6B, and 6C depict data demonstrating in vivo efficacy of <mouse CSF1R> antibody combinations in the MC38 mouse CRC in vivo model.

FIG. 7 depicts data demonstrating in vivo efficacy of <CSF1R> antibody and <CD40> combination: Combination of CSF1R mAb+CD40 mAb FGK45 shows improved anti-tumor efficacy over monotherapies in syngeneic MC38 mouse colon cancer model.

### DETAILED DESCRIPTION OF THE INVENTION

Many tumors are characterized by a prominent immune cell infiltrate, including macrophages. Initially, the immune cells were thought to be part of a defense mechanism against the tumor, but recent data support the notion that several immune cell populations including macrophages may, in fact, promote tumor progression. Macrophages are characterized by their plasticity. Depending on the cytokine microenvironment, macrophages can exhibit so-called M1 or M2-subtypes. M2 macrophages are engaged in the suppression of tumor immunity. They also play an important role in tissue repair functions such as angiogenesis and tissue remodeling which are coopted by the tumor to support growth. In contrast to tumor promoting M2 macrophages, M1 macrophages exhibit antitumor activity via the secretion of inflammatory cytokines and their engagement in antigen presentation and phagocytosis (Mantovani, A. et al., Curr. Opin. Immunol. 2 (2010) 231-237).

By secreting various cytokines such as colony stimulating factor 1 (CSF-1) and IL-10, tumor cells are able to recruit and shape macrophages into the M2-subtype, whereas cytokines such as granulocyte macrophage colony stimulating factor (GM-CSF), IFN-gamma program macrophages towards the M1 subtype. Using immunohistochemistry, it is possible to distinguish between a macrophage subpopulation co-expressing CD68 and CD163, which is likely to be enriched for M2 Macrophages, and a subset showing the CD68+/MHC II+, or CD68+/CD80+ immunophenotype, likely to include M1 macrophages. Cell shape, size, and spatial distribution of CD68 and CD163 positive macrophages is consistent with published hypotheses on a tumor-promoting role of M2 macrophages, for example by their preferential location in tumor intersecting stroma, and vital tumor areas. In contrast, CD68+/MHC class II+ macrophages are ubiquitously found. Their hypothetical role in phagocytosis is reflected by clusters of the CD68+/MHC class II+, but CD163-immunophenotype near apoptotic cells and necrotic tumor areas. The subtype and marker expression of different macrophage subpopulations is linked with their functional state. M2 macrophages can support tumorigenesis by:
a) enhancing angiogenesis via the secretion of angiogenic factors such as VEGF or bFGF,
b) supporting metastasis formation via secretion of matrix metalloproteinases (MMPs), growth factors and migratory factors guiding the tumor cells to the blood stream and setting up the metastatic niche (Wyckoff, J. et al., Cancer Res. 67 (2007) 2649-2656),
c) playing a role in building an immunosuppressive milieu by secreting immunosuppressive cytokines such as IL-4, Il-13, IL-1ra and IL-10, which in turn regulate T regulatory cell function. Conversely CD4 positive T cells have been shown to enhance the activity of tumor promoting macrophages in preclinical models (Mantovani, A. et al., Eur. J. Cancer 40 (2004) 1660-1667; DeNardo, D. et al., Cancer Cell 16 (2009) 91-102).

Accordingly, in several types of cancer (e.g. breast, ovarian, Hodgkin's lymphoma) the prevalence of M2 subtype tumor associated macrophages (TAMs) has been associated with poor prognosis (Bingle, L. et al., J. Pathol. 3 (2002) 254-265; Orre, M., and Rogers, P. A., Gynecol. Oncol. 1 (1999) 47-50; Steidl, C. et al., N. Engl. J. Med. 10 (2010) 875-885). Recent data show a correlation of CD163 positive macrophage infiltrate in tumors and tumor grade (Kawamura, K. et al., Pathol. Int. 59 (2009) 300-305). TAMs isolated from patient tumors had a tolerant phenotype and were not cytotoxic to tumor cells (Mantovani, A. et al., Eur. J. Cancer 40 (2004) 1660-1667). However, infiltration of TAMs in the presence of cytotoxic T cells correlates with improved survival in non small cell lung cancer and hence reflects a more prominent M1 macrophage infiltrate in this tumor type (Kawai, O. et al., Cancer 6 (2008) 1387-1395).

Recently, a so-called immune signature comprising high numbers of macrophages and CD4 positive T cells, but low numbers of cytotoxic CD8 positive T cells was shown to correlate with reduced overall survival (OS) in breast cancer patients and to represent an independent prognostic factor (DeNardo, D. et al., Cancer Discovery 1 (2011) 54-67).

Consistent with a role for CSF-1 in driving the pro-tumorigenic function of M2 macrophages, high CSF-1 expression in rare sarcomas or locally aggressive connective tissue tumors, such as pigmented villonodular synovitis (PVNS) and tenosynovial giant cell tumor (TGCT) due in part to a translocation of the CSF-1 gene, leads to the accumulation of monocytes and macrophages expressing the receptor for CSF-1, the colony-stimulating factor 1 receptor (CSF-1R) forming the majority of the tumor mass (West, R. B. et al., Proc. Natl. Acad. Sci. USA 3 (2006) 690-695). These tumors were subsequently used to define a CSF-1 dependent macrophage signature by gene expression profiling. In breast cancer and leiomyosarcoma patient tumors this CSF-1 response gene signature predicts poor prognosis (Espinosa, I. et al., Am. J. Pathol. 6 (2009) 2347-2356; Beck, A. et al., Clin. Cancer Res. 3 (2009) 778-787).

CSF-1R belongs to the class III subfamily of receptor tyrosine kinases and is encoded by the c-fms proto-oncogene. Binding of CSF-1 or IL-34 induces receptor dimerization, followed by autophosphorylation and activation of downstream signaling cascades. Activation of CSF-1R regulates the survival, proliferation and differentiation of monocytes and macrophages (Xiong, Y. et al., J. Biol. Chem. 286 (2011) 952-960).

In addition to cells of the monocytic lineage and osteoclasts, which derive from the same hematopoetic precursor as the macrophage, CSF-1R/c-fms has also been found to be expressed by several human epithelial cancers such as ovarian and breast cancer and in leiomyosarcoma and TGCT/PVNS, albeit at lower expression levels compared to macrophages. As with TGCT/PVNS, elevated levels of CSF-1, the ligand for CSF-1R, in serum as well as ascites of ovarian cancer patients have been correlated with poor prognosis (Scholl, S. et al., Br. J. Cancer 62 (1994) 342-346; Price, F. et al., Am. J. Obstet. Gynecol. 168 (1993) 520-527). Furthermore, a constitutively active mutant form of CSF 1R is able to transform NIH3T3 cells, one of the properties of an oncogene (Chambers, S., Future Oncol 5 (2009) 1429-1440). Preclinical models provide validation of CSF-1R as an oncology target. Blockade of CSF-1 as well as CSF-1R activity results in reduced recruitment of TAMs. Chemotherapy resulted in elevated CSF-1 expression in tumor cells leading to enhanced TAM recruitment. Blockade of CSF-1R in combination with paclitaxel resulted in activation of CD8 positive cytotoxic T cells leading to reduced tumor growth and metastatic burden in a spontaneous transgenic breast cancer model (DeNardo, D. et al., Cancer Discovery 1 (2011) 54-67).

The anti-CSF-1R antibodies described in the invention bind to the membrane proximal extracellular domains D4 and D5 which constitute the receptor dimerization interface. They block CSF-1, IL-34 mediated as well as ligand-independent activation of the receptor resulting in induction of apoptosis of M2-like macrophages differentiated in vitro in the presence of CSF-1 while sparing the M1-like GM-CSF differentiated macrophages. In human breast cancer tissue, M2 (CD68+/CD163+) macrophages and CSF 1R-expressing macrophages are co-localized. In the cynomolgous monkey 13 week treatment with hMab 2F11-e7 reduced CD163 positive macrophages in the liver and colon but not the macrophages of the lung.

Despite the introduction of several new agents, the clinical management of many advanced solid tumors remains challenging. Advances in the understanding of molecular cancer biology have stimulated research into more targeted therapies with the aim of improving the outcome.

CSF-1R is a protein encoded by the CSF-1R gene. It controls the production, differentiation, and function of M2 macrophages, which, in turn, support tumor growth and metastasis formation and secrete immunosuppressive cytokines, leading to a poor prognosis in patients. Furthermore, presence of CSF-1R positive macrophages in several human cancers (such as ovarian and breast carcinoma) has been shown to correlate not only with increased vascular density but also worse clinical outcome. CSF-1R inhibitors, which selectively inhibit M2-like TAMs, have demonstrated activity in preclinical models (DeNardo, D. et al., Cancer Discovery 1 (2011) 54-67; Lin, E. et al., J. Exp. Med. 193 (2001) 727-740). Blockade of CSF-1R activity results in reduced recruitment of TAMs and, in combination with chemotherapy, a synergistic action results in reduced tumor growth and metastatic burden. Recent data have shown that in patients with PVNS and TGCT, overexpression of the CSF-1 is detected and is in part mediated by a translocation of the CSF-1R gene (West, R. B. et al., Proc. Natl. Acad. Sci. USA 3 (2006) 690-695). In breast cancer the presence of a CSF-1 response gene signature predicts risk of recurrence and metastasis (Beck, A. et al., Clin. Cancer Res. 3 (2009) 778-787).

Based on the antitumor single agent efficacy of the antibodies described in the invention, it seems reasonable to test the hypothesis that blockade of tumor associated macrophages and their pro-tumor bioactivity in combination with taxanes (like e.g. paclitaxel (Taxol), docetaxel (Taxotere), modified paclitaxel (e.g., Abraxane and Opaxio), doxorubicin, sunitinib (Sutent), sorafenib (Nexavar), and other multikinase inhibitors, oxaliplatin, oxaliplatin, cisplatin and carboplatin, etoposide, gemcitabine, and vinblastine. In one embodiment the chemotherapeutic agent is selected from the group consisting of taxanes (like e.g. taxol (paclitaxel), docetaxel (Taxotere), modified paclitaxel (e.g. Abraxane and Opaxio).

The invention comprises the combination therapy with an antibody binding to human CSF-1R, characterized in that the antibody binds to human CSF-1R Extracellular Domain (SEQ ID NO: 64) (comprising domains D1 to D5) and does not bind to domains D1 to D3 (SEQ ID NO: 66) of the extracellular domain of human CSF-1R.

The invention further comprises the combination therapy with an antibody binding to human CSF-1R, characterized in that the antibody binds to human CSF-1R fragment delD4 (comprising the extracellular subdomains D1-D3 and D5) (SEQ ID NO: 65) and to human CSF-1R Extracellular Domain (CSF-1R-ECD) (comprising the extracellular subdomains D1-D5) (SEQ ID NO: 64) with a ratio of 1:50 or lower.

The invention further comprises the combination therapy with an antibody binding to human CSF-1R, characterized in comprising as heavy chain variable domain CDR3 region a CDR3 region of SEQ ID NO: 1, SEQ ID NO: 9, SEQ ID NO:23, SEQ ID NO:31, SEQ ID NO:39, SEQ ID NO:47 or SEQ ID NO:55.

The invention further comprises the combination therapy with an antibody binding to human CSF-1R, characterized in that
a) the heavy chain variable domain is SEQ ID NO:7 and the light chain variable domain is SEQ ID NO:8,
b) the heavy chain variable domain is SEQ ID NO:15 and the light chain variable domain is SEQ ID NO:16;
   or a humanized version thereof.

The invention further comprises the combination therapy with an antibody binding to human CSF-1R, characterized in that
a) the heavy chain variable domain is SEQ ID NO:7 and the light chain variable domain is SEQ ID NO:8,
b) the heavy chain variable domain is SEQ ID NO:15 and the light chain variable domain is SEQ ID NO:16;
c) the heavy chain variable domain is SEQ ID NO:75 and the light chain variable domain is SEQ ID NO:76;
d) the heavy chain variable domain is SEQ ID NO:83 and the light chain variable domain is SEQ ID NO:84;
   or a humanized version thereof.

The invention further comprises the combination therapy with an antibody binding to human CSF-1R, characterized in that
the heavy chain variable domain is SEQ ID NO:7 and the light chain variable domain is SEQ ID NO:8, or a humanized version thereof.

In one embodiment the combination therapy with an antibody binding to human CSF-1R, is characterized in that
a) the heavy chain variable domain is SEQ ID NO:23 and the light chain variable domain is SEQ ID NO:24, or
b) the heavy chain variable domain is SEQ ID NO:31 and the light chain variable domain is SEQ ID NO:32, or
c) the heavy chain variable domain is SEQ ID NO:39 and the light chain variable domain is SEQ ID NO:40, or
d) the heavy chain variable domain is SEQ ID NO:47 and the light chain variable domain is SEQ ID NO:48, or
e) the heavy chain variable domain is SEQ ID NO:55 and the light chain variable domain is SEQ ID NO:56.

In one embodiment the combination therapy with an antibody binding to human CSF-1R, is characterized in that
a) the heavy chain variable domain is SEQ ID NO:23 and the light chain variable domain is SEQ ID NO:24, or
b) the heavy chain variable domain is SEQ ID NO:31 and the light chain variable domain is SEQ ID NO:32, or
c) the heavy chain variable domain is SEQ ID NO:39 and the light chain variable domain is SEQ ID NO:40, or
d) the heavy chain variable domain is SEQ ID NO:47 and the light chain variable domain is SEQ ID NO:48.

In one embodiment the antibody according to the invention is characterized in that
the heavy chain variable domain is SEQ ID NO:23 and the light chain variable domain is SEQ ID NO:24.

In one embodiment the combination therapy with an antibody binding to human CSF-1R, is characterized in that
the heavy chain variable domain is SEQ ID NO:31 and the light chain variable domain is SEQ ID NO:32.

In one embodiment the combination therapy with an antibody binding to human CSF-1R, is characterized in that
the heavy chain variable domain is SEQ ID NO:39 and the light chain variable domain is SEQ ID NO:40.

In one embodiment the combination therapy with an antibody binding to human CSF-1R, is characterized in that
the heavy chain variable domain is SEQ ID NO:47 and the light chain variable domain is SEQ ID NO:48.

The invention further comprises the combination therapy with an antibody binding to human CSF-1R, characterized in that
the heavy chain variable domain is SEQ ID NO:15 and the light chain variable domain is SEQ ID NO:16, or a humanized version thereof.

The invention further comprises the combination therapy with an antibody binding to human CSF-1R, characterized in that
the heavy chain variable domain is SEQ ID NO:75 and the light chain variable domain is SEQ ID NO:76;
   or a humanized version thereof.

The invention further the combination therapy with an antibody binding to human CSF-1R, characterized in that
the heavy chain variable domain is SEQ ID NO:83 and the light chain variable domain is SEQ ID NO:84;
   or a humanized version thereof.

The invention further the combination therapy with an antibody binding to human CSF-1R, characterized in that
a) the heavy chain variable domain comprises a CDR3 region of SEQ ID NO:1, a CDR2 region of SEQ ID NO: 2, and a CDR1 region of SEQ ID NO:3, and the light chain variable domain comprises a CDR3 region of SEQ ID NO: 4, a CDR2 region of SEQ ID NO:5, and a CDR1 region of SEQ ID NO:6, or,
b) the heavy chain variable domain comprises a CDR3 region of SEQ ID NO: 9, a CDR2 region of SEQ ID NO: 10, and a CDR1 region of SEQ ID NO: 11, and the light chain variable domain comprises a CDR3 region of SEQ ID NO:12, a CDR2 region of SEQ ID NO: 13, and a CDR1 region of SEQ ID NO: 14, or
c) the heavy chain variable domain comprises a CDR3 region of SEQ ID NO: 17, a CDR2 region of SEQ ID NO: 18, and a CDR1 region of SEQ ID NO:19, and the light chain variable domain comprises a CDR3 region of SEQ ID NO: 20, a CDR2 region of SEQ ID NO:21, and a CDR1 region of SEQ ID NO:22, or
d) the heavy chain variable domain comprises a CDR3 region of SEQ ID NO: 25, a CDR2 region of SEQ ID NO: 26, and a CDR1 region of SEQ ID NO: 27, and the light chain variable domain comprises a CDR3 region of SEQ ID NO:28, a CDR2 region of SEQ ID NO: 29, and a CDR1 region of SEQ ID NO: 30, or
e) the heavy chain variable domain comprises a CDR3 region of SEQ ID NO: 33, a CDR2 region of SEQ ID NO: 34, and a CDR1 region of SEQ ID NO: 35, and the light chain variable domain comprises a CDR3 region of SEQ ID NO:36, a CDR2 region of SEQ ID NO: 37, and a CDR1 region of SEQ ID NO: 38, or
f) the heavy chain variable domain comprises a CDR3 region of SEQ ID NO:41, a CDR2 region of SEQ ID NO: 42, and a CDR1 region of SEQ ID NO:43, and the light chain variable domain comprises a CDR3 region of SEQ ID NO: 44, a CDR2 region of SEQ ID NO:45, and a CDR1 region of SEQ ID NO:46, or
g) the heavy chain variable domain comprises a CDR3 region of SEQ ID NO: 49, a CDR2 region of SEQ ID NO: 50, and a CDR1 region of SEQ ID NO: 51, and the light chain variable domain comprises a CDR3 region of SEQ ID NO:52, a CDR2 region of SEQ ID NO: 53, and a CDR1 region of SEQ ID NO: 54.

The invention further comprises the combination therapy with an antibody binding to human CSF-1R, characterized in that
a) the heavy chain variable domain comprises a CDR3 region of SEQ ID NO:1, a CDR2 region of SEQ ID NO: 2, and a CDR1 region of SEQ ID NO:3, and the light chain variable domain comprises a CDR3 region of SEQ ID NO: 4, a CDR2 region of SEQ ID NO:5, and a CDR1 region of SEQ ID NO:6, or
b) the heavy chain variable domain comprises a CDR3 region of SEQ ID NO: 9, a CDR2 region of SEQ ID NO: 10, and a CDR1 region of SEQ ID NO: 11, and the light chain variable domain comprises a CDR3 region of SEQ ID NO:12, a CDR2 region of SEQ ID NO: 13, and a CDR1 region of SEQ ID NO: 14, or
c) the heavy chain variable domain comprises a CDR3 region of SEQ ID NO: 17, a CDR2 region of SEQ ID NO: 18, and a CDR1 region of SEQ ID NO:19, and the light chain variable domain comprises a CDR3 region of SEQ ID NO: 20, a CDR2 region of SEQ ID NO:21, and a CDR1 region of SEQ ID NO:22, or
d) the heavy chain variable domain comprises a CDR3 region of SEQ ID NO: 25, a CDR2 region of SEQ ID NO: 26, and a CDR1 region of SEQ ID NO: 27, and the light chain variable domain comprises a CDR3 region of SEQ ID NO:28, a CDR2 region of SEQ ID NO: 29, and a CDR1 region of SEQ ID NO: 30, or
e) the heavy chain variable domain comprises a CDR3 region of SEQ ID NO: 33, a CDR2 region of SEQ ID NO: 34, and a CDR1 region of SEQ ID NO: 35, and the light chain variable domain comprises a CDR3 region of SEQ ID NO:36, a CDR2 region of SEQ ID NO: 37, and a CDR1 region of SEQ ID NO: 38, or
f) the heavy chain variable domain comprises a CDR3 region of SEQ ID NO:41, a CDR2 region of SEQ ID NO: 42, and a CDR1 region of SEQ ID NO:43, and the light chain variable domain comprises a CDR3 region of SEQ ID NO: 44, a CDR2 region of SEQ ID NO:45, and a CDR1 region of SEQ ID NO:46, or
g) the heavy chain variable domain comprises a CDR3 region of SEQ ID NO: 49, a CDR2 region of SEQ ID NO: 50, and a CDR1 region of SEQ ID NO: 51, and the light chain variable domain comprises a CDR3 region of SEQ ID NO:52, a CDR2 region of SEQ ID NO: 53, and a CDR1 region of SEQ ID NO: 54; or
h) the heavy chain variable domain comprises a CDR3 region of SEQ ID NO:69, a CDR2 region of SEQ ID NO: 70, and a CDR1 region of SEQ ID NO:71, and the light chain variable domain comprises a CDR3 region of SEQ ID NO: 72, a CDR2 region of SEQ ID NO:73, and a CDR1 region of SEQ ID NO:74, or
i) the heavy chain variable domain comprises a CDR3 region of SEQ ID NO: 77, a CDR2 region of SEQ ID NO: 78, and a CDR1 region of SEQ ID NO: 79, and the light chain variable domain comprises a CDR3 region of SEQ ID NO:80, a CDR2 region of SEQ ID NO: 81, and a CDR1 region of SEQ ID NO: 82.

In one embodiment the combination therapy with an antibody binding to human CSF-1R, is characterized in that
a) the heavy chain variable domain comprises a CDR3 region of SEQ ID NO:69, a CDR2 region of SEQ ID NO: 70, and a CDR1 region of SEQ ID NO:71, and the light chain variable domain comprises a CDR3 region of SEQ ID NO: 72, a CDR2 region of SEQ ID NO:73, and a CDR1 region of SEQ ID NO:74, or
b) the heavy chain variable domain comprises a CDR3 region of SEQ ID NO: 77, a CDR2 region of SEQ ID NO: 78, and a CDR1 region of SEQ ID NO: 79, and the light chain variable domain comprises a CDR3 region of SEQ ID NO:80, a CDR2 region of SEQ ID NO: 81, and a CDR1 region of SEQ ID NO: 82.

In one embodiment the combination therapy with an antibody binding to human CSF-1R, is characterized in that
a) the heavy chain variable domain comprises a CDR3 region of SEQ ID NO: 17, a CDR2 region of SEQ ID NO: 18, and a CDR1 region of SEQ ID NO:19, and the light chain variable domain comprises a CDR3 region of SEQ ID NO: 20, a CDR2 region of SEQ ID NO:21, and a CDR1 region of SEQ ID NO:22, or
b) the heavy chain variable domain comprises a CDR3 region of SEQ ID NO: 25, a CDR2 region of SEQ ID NO: 26, and a CDR1 region of SEQ ID NO: 27, and the light chain variable domain comprises a CDR3 region of SEQ ID NO:28, a CDR2 region of SEQ ID NO: 29, and a CDR1 region of SEQ ID NO: 30, or
c) the heavy chain variable domain comprises a CDR3 region of SEQ ID NO: 33, a CDR2 region of SEQ ID NO: 34, and a CDR1 region of SEQ ID NO: 35, and the light chain variable domain comprises a CDR3 region of SEQ ID NO:36, a CDR2 region of SEQ ID NO: 37, and a CDR1 region of SEQ ID NO: 38, or
d) the heavy chain variable domain comprises a CDR3 region of SEQ ID NO:41, a CDR2 region of SEQ ID NO: 42, and a CDR1 region of SEQ ID NO:43, and the light chain variable domain comprises a CDR3 region of SEQ ID NO: 44, a CDR2 region of SEQ ID NO:45, and a CDR1 region of SEQ ID NO:46, or
e) the heavy chain variable domain comprises a CDR3 region of SEQ ID NO: 49, a CDR2 region of SEQ ID NO: 50, and a CDR1 region of SEQ ID NO: 51, and the light chain variable domain comprises a CDR3 region of SEQ ID NO:52, a CDR2 region of SEQ ID NO: 53, and a CDR1 region of SEQ ID NO: 54.

In one embodiment the combination therapy with an antibody binding to human CSF-1R, is characterized in that
a) the heavy chain variable domain comprises a CDR3 region of SEQ ID NO: 17, a CDR2 region of SEQ ID NO: 18, and a CDR1 region of SEQ ID NO:19, and the light chain variable domain comprises a CDR3 region of SEQ ID NO: 20, a CDR2 region of SEQ ID NO:21, and a CDR1 region of SEQ ID NO:22, or
b) the heavy chain variable domain comprises a CDR3 region of SEQ ID NO: 25, a CDR2 region of SEQ ID NO: 26, and a CDR1 region of SEQ ID NO: 27, and the light chain variable domain comprises a CDR3 region of SEQ ID NO:28, a CDR2 region of SEQ ID NO: 29, and a CDR1 region of SEQ ID NO: 30, or
c) the heavy chain variable domain comprises a CDR3 region of SEQ ID NO: 33, a CDR2 region of SEQ ID NO: 34, and a CDR1 region of SEQ ID NO: 35, and the light chain variable domain comprises a CDR3 region of SEQ ID NO:36, a CDR2 region of SEQ ID NO: 37, and a CDR1 region of SEQ ID NO: 38, or
d) the heavy chain variable domain comprises a CDR3 region of SEQ ID NO:41, a CDR2 region of SEQ ID NO: 42, and a CDR1 region of SEQ ID NO:43, and the light chain variable domain comprises a CDR3 region of SEQ ID NO: 44, a CDR2 region of SEQ ID NO:45, and a CDR1 region of SEQ ID NO:46.

In one embodiment the combination therapy with an antibody binding to human CSF-1R, is characterized in that
the heavy chain variable domain comprises a CDR3 region of SEQ ID NO: 17, a CDR2 region of SEQ ID NO: 18, and a CDR1 region of SEQ ID NO:19, and the light chain variable domain comprises a CDR3 region of SEQ ID NO: 20, a CDR2 region of SEQ ID NO:21, and a CDR1 region of SEQ ID NO:22.

In one embodiment the combination therapy with an antibody binding to human CSF-1R, is characterized in that
the heavy chain variable domain comprises a CDR3 region of SEQ ID NO: 25, a CDR2 region of SEQ ID NO: 26, and a CDR1 region of SEQ ID NO: 27, and the light chain variable domain comprises a CDR3 region of SEQ ID NO:28, a CDR2 region of SEQ ID NO: 29, and a CDR1 region of SEQ ID NO: 30.

In one embodiment the combination therapy with an antibody binding to human CSF-1R, is characterized in that
the heavy chain variable domain comprises a CDR3 region of SEQ ID NO: 33, a CDR2 region of SEQ ID NO: 34, and a CDR1 region of SEQ ID NO: 35, and the light chain variable domain comprises a CDR3 region of SEQ ID NO:36, a CDR2 region of SEQ ID NO: 37, and a CDR1 region of SEQ ID NO: 38.

In one embodiment the combination therapy with an antibody binding to human CSF-1R, is characterized in that
the heavy chain variable domain comprises a CDR3 region of SEQ ID NO:41, a CDR2 region of SEQ ID NO: 42, and a CDR1 region of SEQ ID NO:43, and the light chain variable domain comprises a CDR3 region of SEQ ID NO: 44, a CDR2 region of SEQ ID NO:45, and a CDR1 region of SEQ ID NO:46.

In one embodiment the antibody binding to human CSF-1R, characterized in that the antibody binds to human CSF-1R fragment delD4 (SEQ ID NO: 65) and to human CSF-1R-ECD (SEQ ID NO: 64) with a ratio of 1:50 or lower, is further characterized in not binding to human CSF-1R fragment D1-D3 (SEQ ID NO: 66). Another aspect of the invention is the selection of patients which are likely to benefit of from treatment with an anti-CSF-1R antibody (including all CSF-1R antibodies binding to human CSF-1R) (administered either alone or in combination with a chemotherapeutic agent, or a cancer immunotherapy, or irradiation, (including all CSF-1R antibodies binding to human CSF-1R). In one embodiment such patient selection relates to treatment with CSF-1R antibodies binding to the domains D4 to D5 of the extracellular domain of human CSF-1R binding to the domains D4 to D5 of the extracellular domain. One or more of the following biomarkers are useful in such a method for the selection of a patient who is likely to responds to such treatment.

### Rationale for Biomarker Evaluation

Biomarkers have the potential to shape diagnostic strategies and influence therapeutic management. In the future, biomarkers Biomarkers may promote a personalized medicine approach, e.g. leading to a grouping of patients by the molecular signatures of their tumors and of markers in their blood rather than by cancer type. We are concentrating our efforts in identifying predictive biomarkers, which provide information about the likely efficacy and safety of the therapy. To evaluate the PD and mechanistic effect/s of a drug on the tumor a tumor biopsy is often required.

### Rationale for Fresh Pre- and On-Treatment Tumor Biopsy in Clinical Testing

TAM infiltration and differentiation is dependent on the respective tumor micro-milieu in primary and metastatic lesions. Furthermore the respective immune status and pre-treatment of the patient might can influence the patient's tumor microenvironment. Therefore all patients will undergo a mandatory pre-treatment biopsy to define the TAM infiltration and CSF-1R expression levels at baseline but will not be used to determine patient eligibility for the trial. In addition, mandatory on-treatment biopsies will allow for the assessment of the PD activity of CSF-1R antibodies by comparing CSF-1R, CD68/CD163, CD68/MHC class II, CD31 (microvessel density), Ki67 and other immune infiltrating cells (e.g. T cells) pre- and post-dose levels. Fine Needle Aspiration (FNA) will not be not suitable to substitute for tumor biopsies, as macrophage sub-population distribution needs to be assessed in the tissue.

Archival tumor tissue cannot substitute for the fresh biopsies as macrophage infiltration and differentiation is micro-milieu dependent. The tumor micro-milieu may be variable in the primary tumor due to pre-treatment of the patient and as well be altered in metastatic lesions. However, if archival tumor tissue is available, submission to Clinical Sample Operations (CSO) is encouraged. Samples will be used for exploratory retrospective correlation of data with fresh biopsies.

### Rationale for Wounded Skin Biopsies in Clinical Testing

The different phases of wound healing require many processes (e.g. neutrophil recruitment, macrophage infiltration, angiogenesis (Eming, S. A., et al., Frog. Histochem. Cytochem. 42 (2007) 115-170)). Skin wounding assays have been used to obtain surrogate tissue to determine PD markers for e.g. anti-angiogenic therapies (Zhang, D. et al., Invest. New Drugs 25 (2006) 49-55; Lockhart, A. C. et al., Clin. Cancer Res. 9 (2003) 586-593). During wound healing macrophages play a substantial role and phenotypic changes of wound associated macrophages (WAM) account for the different roles in the phases of skin repair (e.g. early inflammatory phase=intense phagocytic activity; mid tissue remodelling phase: immunoregulatory state with overexpression of pro-angiogenic factors) (Adamson, R., Journal of Wound Care 18 (2009) 349-351; Rodero, M. P. et al., Int. J. Clin. Exp. Pathol. 25 (2010) 643-653; Brancato, S. K. and Albina, J. E., Wound Macrophages as Key Regulators of Repair, Origin, Phenotype, and Function, AJP (2011) Vol. 178, No. 1).

Indeed, the absence of macrophages resulted in delayed wound healing in genetically engineered mice (Rodero, M. P. et al., Int. J. Clin. Exp. Pathol. 25 (2010) 643-653). Preclinical experiments showed a significant (F4/80 positive) macrophage reduction in the skin of a CSF-1R treated MDA-MB231 xenograft mouse model. However, species specific differences between mouse and human have been reported (Daley, J. M. et al., J. Leukoc. Biol. 87 (2009) 1-9). As WAMs and TAMs are originating from the same progenitor cells and share similar functions and phenotypes, serial pre-treatment and on-treatment (total of n=4) skin biopsies will can be used to analyze the pharmacodynamics effects of CSF-1R antibody treatment on WAMs during the wound healing process. Correlation of the skin data with PD effects of CSF-1R antibody treatment on TAMs in fresh tumor biopsies can significantly increase knowledge on the molecular basis of how CSF-1R antibody works and how the tumor is responding. In addition, the assessment of wounded skin tissue macrophages might potentially substitute for the on-treatment tumor biopsies. In later trials the assessment of WAMs therefore may serve as surrogate tissue to in the assessment of CSF-1R antibody efficacy.

### Rationale for Measurement of Biomarkers in Whole Blood Samples to Measure Biomarkers or PD Markers

Preclinical experiments have shown that changes in e.g. circulating CSF-1, TRAP5b monocyte subpopulations and tissue macrophages are associated with the drug activity of anti-CSF-1R therapeutic agents. In addition, GLP-Tox data from CSF-1R antibody treated cynomolgous monkeys revealed alterations in biomarkers of bone formation (osteocalcin, P1NP), osteoclast activity (TRAP5b) and parathyroid hormone which all correlated with bone metabolism.

Therefore, these markers and additional circulating immunostimulatory or immunoinhibitory factors as well as e.g. soluble CD163 (to monitor the activation of monocytes/macrophages) can be useful to monitor pharmacodynamic changes and for selection of patients who are likely to respond favorably to an anti-CSF-1R antibody treatment.

These surrogate tissue specimens will be used for research purposes to identify biomarkers that are predictive of response to CSF-1R antibody treatment (in terms of dose, safety and tolerability) and will help to better understand the pathogenesis, course and outcome of cancer and related diseases. Analysis may include determination of circulating markers associated with the PD activity of CSF-1R antibodies (e.g. assessment of cytokine levels, circulating immune cells and immune effector cell depletion). Preclinical experiments have shown that changes in e.g. circulating CSF-1, TRAP5b monocyte subpopulations and tissue macrophages are associated with the drug activity. In addition, GLP-Tox data from CSF-1R antibody treated cynomolgous monkeys revealed alterations in bone biomarkers of formation (osteocalcin, P1NP), osteoclast activity (TRAP5b) and parathyroid hormone which all correlated with reduced osteoclast numbers. Therefore, these markers and additional circulating immunostimulatory or immunoinhibitory factors can be useful for selection patients who will respond favorably to an anti-CSF-1R antibody treatment.

One aspect of the present invention is a method for determining whether a subject having a cancer is a candidate for an anti-CSF-1R antibody-based cancer treatment regimen, wherein said antibody is the antibody of the present invention comprising:
ex vivo or in vitro determining in vitro the level of one or more of the following markers:
CSF-1R, CD68/CD163, CD68/MHC class II, CD31 (microvessel density), and Ki67 and other markers like e g immuninfiltrates;
in a sample of the subject, wherein the sample is selected from the group consisting of tissue, blood, serum, plasma, tumor cells and circulating tumor cells; and
wherein an change in the level of one or more of CSF-1R, CD68/CD163, CD68/MHC class II, CD31 (microvessel density) and Ki67 and other markers like e.g. immuninfiltrates (e.g. T cells (e.g. CD4- and/or CD8-T cells), as compared with to the corresponding level in an individual not suffering from cancer, is indicative that the subject is a candidate for the anti-CSF-1 R antibody-based cancer treatment regimen.
In one embodiment this method is practiced for an anti-CSF-1R antibody-based cancer treatment regimen, wherein the antibody used in said regimen is an antibody according to the present invention.
In one embodiment of this method the change in the level of CSF-1R, CD68/CD163, CD68/MHC class II, CD31 (microvessel density) and Ki67 and other markers like e.g. immuninfiltrates (e.g. T cells (e.g. CD4- and/or CD8-T cells), as compared to the level in an individual not suffering from cancer is an increase in the level of one or more of these markers.
One aspect of the present invention is a method for determining whether a subject having a cancer is a candidate for an anti-CSF-1R antibody-based cancer treatment regimen, wherein said antibody is the antibody of the present invention comprising:
ex vivo or in vitro determining the level of one or more of the following markers:
CSF-1R, CD68/CD163, CD68/MHC class II, CD31 (microvessel density) and Ki67;
in a sample of the subject, wherein the sample is selected from the group consisting of blood, serum, plasma, tumor cells and circulating tumor cells; and
wherein a change in the level of CSF-1R, CD68/CD163, CD68/MHC class II, CD31 (microvessel density) and Ki67, as compared with the corresponding level in an individual not suffering from cancer, is indicative that the subject is a candidate for the anti-CSF-1 R antibody-based cancer treatment regimen.
One aspect of the present invention is a method for determining whether a subject having a cancer is a candidate for an anti-CSF-1R antibody-based cancer treatment regimen, wherein said antibody is the antibody of the present invention comprising:
ex vivo or in vitro determining in vitro the level of one or more of the following markers:
CSF-1, Trap5b, sCD163, IL-34;
in a sample of the subject, wherein the sample is selected from the group consisting of blood, serum, plasma, tumor cells (e.g. in form of a sample of the tumor tissue) and circulating tumor cells; and
wherein an change in the level of one or more of CSF-1, Trap5b, sCD163, IL-34, as compared with to the corresponding level in an individual not suffering from cancer, is indicative that the subject is a candidate for the anti-CSF-1 R antibody-based cancer treatment regimen.
In one embodiment this method is practiced for an anti-CSF-1R antibody-based cancer treatment regimen, wherein the antibody used in said regimen is an antibody according to the present invention.
In one embodiment of this method the change in the level of CSF-1, Trap5b, sCD163, IL-34, as compared to the level in an individual not suffering from cancer is a change in the level of one or more of these markers.
One aspect of the present invention is a method for determining whether a subject having a cancer is a candidate for an anti-CSF-1R antibody-based cancer treatment regimen, wherein said antibody is the antibody of the present invention comprising:
ex vivo or in vitro determining the level of one or more of the following markers:
CSF-1, Trap5b, sCD163, IL-34;
in a sample of the subject, wherein the sample is selected from the group consisting of blood, serum, plasma, tumor cells and circulating tumor cells; and
wherein a change in the level of CSF-1, Trap5b, sCD163, IL-34, as compared with the corresponding level in an individual not suffering from cancer, is indicative that the subject is a candidate for the anti-CSF-1 R antibody-based cancer treatment regimen.
One aspect of the present invention is a method for determining whether a subject having a cancer is a candidate for an anti-CSF-1R antibody-based cancer treatment regimen, wherein said antibody is the antibody of the present invention comprising:
ex vivo or in vitro determining in vitro the level of one or more of the following markers:
sCD163;
in a sample of the subject, wherein the sample is selected from the group consisting of blood, serum, plasma, tumor cells and circulating tumor cells; and
wherein an change in the level of sCD163 as compared with to the corresponding level in an individual not suffering from cancer, is indicative that the subject is a candidate for the anti-CSF-1 R antibody-based cancer treatment regimen.
In one embodiment this method is practiced for an anti-CSF-1R antibody-based cancer treatment regimen, wherein the antibody used in said regimen is an antibody according to the present invention.
In one embodiment of this method the change in the level of sCD163 as compared to the level in an individual not suffering from cancer is an increase in the level of this markers.
One aspect of the present invention is a method for determining whether a subject having a cancer is a candidate for an anti-CSF-1R antibody-based cancer treatment regimen, wherein said antibody is the antibody of the present invention comprising:
ex vivo or in vitro determining the level of one or more of the following markers:
sCD163;
in a sample of the subject, wherein the sample is selected from the group consisting of blood, serum, plasma, tumor cells and circulating tumor cells; and
wherein a change in the level of sCD163 as compared with the corresponding level in an individual not suffering from cancer, is indicative that the subject is a candidate for the anti-CSF-1 R antibody-based cancer treatment regimen.
One aspect of the present invention is a method for determining whether a subject having a cancer is a candidate for an anti-CSF-1R antibody-based cancer treatment regimen, wherein said antibody is the antibody of the present invention comprising:
ex vivo or in vitro determining in vitro the level of one or more of the following markers: IFNγ, TNFα, IL-1β, IL-4, IL-6, IL-8, IL-10, IL-13, GM-CSF, VEGF, MCP-1, CCL18, CCL22, MIP-1, Galectin 3, IL1Ra, TGF alpha;
in a sample of the subject, wherein the sample is selected from the group consisting of blood, serum, plasma, tumor cells and circulating tumor cells; and
wherein a change in the level of one or more of IFNγ, TNFα, IL-1β, IL-4, IL-6, IL-8, IL-10, IL-13, GM-CSF, VEGF, MCP-1, CCL18, CCL22, MIP-1, Galectin 3, IL1Ra, TGF alpha, as compared with to the corresponding level in an individual not suffering from cancer, is indicative that the subject is a candidate for the anti-CSF-1 R antibody-based cancer treatment regimen. In one embodiment this method is practiced for an anti-CSF-1R antibody-based cancer treatment regimen, wherein the antibody used in said regimen is an antibody according to the present invention.
   In one embodiment of this method the change in the level of IFNγ, TNFα, IL-1β, IL-4, IL-6, IL-8, IL-10, IL-13, GM-CSF, VEGF, MCP-1, CCL18, CCL22, MIP-1, Galectin 3, IL1Ra, TGF alpha, as compared to the level in an individual not suffering from cancer is an increase in the level of one or more of these markers.
One aspect of the present invention is a method for determining whether a subject having a cancer is a candidate for an anti-CSF-1R antibody-based cancer treatment regimen, wherein said antibody is the antibody of the present invention comprising:
ex vivo or in vitro determining the level of one or more of the following markers: IFNγ, TNFα, IL-1β, IL-4, IL-6, IL-8, IL-10, IL-13, GM-CSF, VEGF, MCP-1, CCL18, CCL22, MIP-1, Galectin 3, IL1Ra, TGF alpha;
in a sample of the subject, wherein the sample is selected from the group consisting of blood, serum, plasma, tumor cells and circulating tumor cells; and
   wherein a change in the level of IFNγ, TNFα, IL-1β, IL-4, IL-6, IL-8, IL-10, IL-13, GM-CSF, VEGF, MCP-1, CCL18, CCL22, MIP-1, Galectin 3, IL1Ra, TGF alpha, as compared with the corresponding level in an individual not suffering from cancer, is indicative that the subject is a candidate for the anti-CSF-1 R antibody-based cancer treatment regimen.

The term “antibody” encompasses the various forms of antibodies including but not being limited to whole antibodies, antibody fragments, human antibodies, humanized antibodies, chimeric antibodies, T cell epitope depleted antibodies, and further genetically engineered antibodies as long as the characteristic properties according to the invention are retained. “Antibody fragments” comprise a portion of a full length antibody, preferably the variable domain thereof, or at least the antigen binding site thereof. Examples of antibody fragments include diabodies, single-chain antibody molecules, and multispecific antibodies formed from antibody fragments. scFv antibodies are, e.g., described in Houston, J. S., Methods in Enzymol. 203 (1991) 46-88). In addition, antibody fragments comprise single chain polypeptides having the characteristics of a V_{H} domain binding to CSF-1R, namely being able to assemble together with a V_{L} domain, or of a V_{L} domain binding to CSF-1R, namely being able to assemble together with a V_{H} domain to a functional antigen binding site and thereby providing the property.

The terms “monoclonal antibody” or “monoclonal antibody composition” as used herein refer to a preparation of antibody molecules of a single amino acid composition.

The term “chimeric antibody” refers to a monoclonal antibody comprising a variable region, i.e., binding region, from mouse and at least a portion of a constant region derived from a different source or species, usually prepared by recombinant DNA techniques. Chimeric antibodies comprising a mouse variable region and a human constant region are especially preferred. Such rat/human chimeric antibodies are the product of expressed immunoglobulin genes comprising DNA segments encoding rat immunoglobulin variable regions and DNA segments encoding human immunoglobulin constant regions. Other forms of “chimeric antibodies” encompassed by the present invention are those in which the class or subclass has been modified or changed from that of the original antibody. Such “chimeric” antibodies are also referred to as “class-switched antibodies.” Methods for producing chimeric antibodies involve conventional recombinant DNA and gene transfection techniques now well known in the art. See, e.g., Morrison, S. L., et al., Proc. Natl. Acad Sci. USA 81 (1984) 6851-6855; U.S. Pat. No. 5,202,238 and U.S. Pat. No. 5,204,244.

The term “humanized antibody” refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.” See e.g. Riechmann, L., et al., Nature 332 (1988) 323-327; and Neuberger, M. S., et al., Nature 314 (1985) 268-270. Optionally the framework region can be modified by further mutations. Also the CDRs can be modified by one or more mutations to generate antibodies according to the invention e.g. by mutagenesis based upon molecular modeling as described by Riechmann, L., et al., Nature 332 (1988) 323-327 and Queen, C., et al., Proc. Natl. Acad. Sci. USA 86 (1989) 10029-10033, or others. Particularly preferred CDRs correspond to those representing sequences recognizing the antigens noted above for chimeric antibodies. A “humanized version of an antibody according to the invention” (which is e.g. of mouse origin) refers to an antibody, which is based on the mouse antibody sequences in which the V_{H} and V_{L} are humanized by standard techniques (including CDR grafting and optionally subsequent mutagenesis of certain amino acids in the framework region and the CDRs). Preferably such humanized version is chimerized with a human constant region (see e.g. Sequences SEQ ID NO:57-61).

Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

In the following examples the terms “Mab” or “muMab” refer to murine monoclonal antibodies such as Mab 2F11 or Mab 2E10, whereas the term “hMab” refers to humanized monoclonal versions of such murine antibodies such as hMab 2F11-c11, hMab 2F11-d8, hMab 2F11-e7, hMab 2F11-f12, etc.

The term “human antibody”, as used herein, is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Brueggemann, M., et al., Year Immunol. 7 (1993) 33-40). Human antibodies can also be produced in phage display libraries (Hoogenboom, H. R., and Winter, G. J. Mol. Biol. 227 (1992) 381-388; Marks, J. D., et al., J. Mol. Biol. 222 (1991) 581-597). The techniques of Cole, et al., and Boerner, et al., are also available for the preparation of human monoclonal antibodies (Cole, S. P. C., et al., Monoclonal Antibodies and Cancer Therapy, Alan R. Liss, p. 77 (1985); and Boerner, P., et al., J. Immunol. 147 (1991) 86-95). As already mentioned for chimeric and humanized antibodies according to the invention the term “human antibody” as used herein also comprises such antibodies which are modified in the constant region to generate the properties according to the invention, especially in regard to C1q binding and/or FcR binding, e.g. by “class switching” i.e. change or mutation of Fc parts (e.g. from IgG1 to IgG4 and/or IgG1/IgG4 mutation).

The term “recombinant human antibody”, as used herein, is intended to include all human antibodies that are prepared, expressed, created or isolated by recombinant means, such as antibodies isolated from a host cell such as a NS0 or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. The recombinant human antibodies according to the invention have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo. The antibodies according to the invention include, in addition, such antibodies having “conservative sequence modifications”, nucleotide and amino acid sequence modifications which do not affect or alter the above-mentioned characteristics of the antibody according to the invention. Modifications can be introduced by standard techniques known in the art, such as site-directed mutagenesis and PCR-mediated mutagenesis. Conservative amino acid substitutions include ones in which the amino acid residue is replaced with an amino acid residue having a similar side chain Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g. glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in a human anti-CSF-1R antibody can be preferably replaced with another amino acid residue from the same side chain family.

Amino acid substitutions can be performed by mutagenesis based upon molecular modeling as described by Riechmann, L., et al., Nature 332 (1988) 323-327 and Queen, C., et al., Proc. Natl. Acad. Sci. USA 86 (1989) 10029-10033. The human CSF-1R (CSF-1 receptor; synonyms: M-CSF receptor; Macrophage colony-stimulating factor 1 receptor, Fms proto-oncogene, c-fms, SEQ ID NO: 22)) is known since 1986 (Coussens, L., et al., Nature 320 (1986) 277-280). CSF-1R is a growth factor and encoded by the c-fms proto-oncogene (reviewed e.g. in Roth, P. and Stanley, E. R., Curr. Top. Microbiol. Immunol. 181 (1992) 141-167). CSF-1R is the receptor for the CSF-1R ligands CSF-1 (macrophage colony stimulating factor, also called M-CSF) (SEQ ID No.: 86) and IL-34 (SEQ ID No.: 87) and mediates the biological effects of these cytokines (Sherr, C. J., et al., Cell 41 (1985) 665-676; Lin, H., et al., Science 320 (2008) 807-811). The cloning of the colony stimulating factor-1 receptor (also called c-fms) was described for the first time in Roussel, M. F., et al., Nature 325 (1987) 549-552. In that publication, it was shown that CSF-1R had transforming potential dependent on changes in the C-terminal tail of the protein including the loss of the inhibitory tyrosine 969 phosphorylation which binds Cbl and thereby regulates receptor down regulation (Lee, P. S., et al., Embo J. 18 (1999) 3616-3628).

CSF-1R is a single chain, transmembrane receptor tyrosine kinase (RTK) and a member of the family of immunoglobulin (Ig) motif containing RTKs characterized by 5 repeated Ig-like subdomains D1-D5 in the extracellular domain (ECD) of the receptor (Wang, Z., et al Molecular and Cellular Biology 13 (1993) 5348-5359). The human CSF-1R Extracellular Domain (CSF-1R-ECD) (SEQ ID NO: 64) comprises all five extracellular Ig-like subdomains D1-D5. The human CSF-1R fragment delD4 (SEQ ID NO: 65) comprises the extracellular Ig-like subdomains D1-D3 and D5, but is missing the D4 subdomain. The human CSF-1R fragment D1-D3 (SEQ ID NO: 66) comprises the respective subdomains D1-D3. The sequences are listed without the signal peptide MGSGPGVLLL LLVATAWHGQ G (SEQ ID NO: 67). The human CSF-1R fragment D4-D3 (SEQ ID NO: 85) comprises the respective subdomains D4-D3.

Currently two CSF-1R ligands that bind to the extracellular domain of CSF-1R are known. The first one is CSF-1 (colony stimulating factor 1, also called M-CSF, macrophage; human CSF-1, SEQ ID NO: 86) and is found extracellularly as a disulfide-linked homodimer (Stanley, E. R. et al., Journal of Cellular Biochemistry 21 (1983) 151-159; Stanley, E. R. et al., Stem Cells 12 Suppl. 1 (1995) 15-24). The second one is IL-34 (human IL-34; SEQ ID NO: 87) (Hume, D. A., et al, Blood 119 (2012) 1810-1820). Thus in one embodiment the term “CSF-1R ligand” refers to human CSF-1 (SEQ ID NO: 86) and/or human IL-34 (SEQ ID NO: 87). For experiments often the active 149 amino acid (aa) fragment of human CSF-1 (aa 33-181 of SEQ ID NO: 86) is used. This active 149 aa fragment of human CSF-1 (aa 33-181 of SEQ ID NO: 86) is contained in all 3 major forms of CSF-1 and is sufficient to mediate binding to CSF-1R (Hume, D. A., et al, Blood 119 (2012) 1810-1820).

The main biological effects of CSF-1R signaling are the differentiation, proliferation, migration, and survival of hematopoietic precursor cells to the macrophage lineage (including osteoclast). Activation of CSF-1R is mediated by its CSF-1R ligands, CSF-1 (M-CSF) and IL-34. Binding of CSF-1 (M-CSF) to CSF-1R induces the formation of homodimers and activation of the kinase by tyrosine phosphorylation (Li, W. et al, EMBO Journal. 10 (1991) 277-288; Stanley, E. R., et al., Mol. Reprod. Dev. 46 (1997) 4-10).

The intracellular protein tyrosine kinase domain is interrupted by a unique insert domain that is also present in the other related RTK class III family members that include the platelet derived growth factor receptors (PDGFR), stem cell growth factor receptor (c-Kit) and fins-like cytokine receptor (FLT3). In spite of the structural homology among this family of growth factor receptors, they have distinct tissue-specific functions.

CSF-1R is mainly expressed on cells of the monocytic lineage and in the female reproductive tract and placenta. In addition expression of CSF-1R has been reported in Langerhans cells in skin, a subset of smooth muscle cells (Inaba, T., et al., J. Biol. Chem. 267 (1992) 5693-5699), B cells (Baker, A. H., et al., Oncogene 8 (1993) 371-378) and microglia (Sawada, M., et al., Brain Res. 509 (1990) 119-124). Cells with mutant human CSF-1R ((SEQ ID NO: 23) are known to proliferate independently of ligand stimulation.

As used herein, “binding to human CSF-1R” or “specifically binding to human CSF-1R” refers to an antibody specifically binding to the human CSF-1R antigen with a binding affinity of KD-value of 1.0×10⁻⁸ mol/l or lower at 35° C., in one embodiment of a KD-value of 1.0×10⁻⁹ mol/l or lower at 35° C. The binding affinity is determined with a standard binding assay at 35° C., such as surface plasmon resonance technique (BIAcore®, GE-Healthcare Uppsala, Sweden) A method for determining the KD-value of the binding affinity is described in Example 9. Thus an “antibody binding to human CSF-1R” as used herein refers to an antibody specifically binding to the human CSF-1R antigen with a binding affinity of KD 1.0×10⁻⁸ mol/l or lower (preferably 1.0×10⁻⁸ mol/l-1.0×10⁻¹² mol/l) at 35° C., preferably of a KD 1.0×10⁻⁹ mol/l or lower at 35° C. (preferably 1.0×10⁻⁹ mol/l-1.0×10⁻¹² mol/l).

The “binding to human CSF-1R fragment delD4 (SEQ ID NO: 65) and to human CSF-1R Extracellular Domain (SEQ ID NO: 64)” as used herein is measured by a Surface Plasmon Resonance assay (Biacore assay) as described in Example 4. The human CSF-1R fragment delD4 (SEQ ID NO: 65) or human CSF-1R Extracellular Domain (SEQ ID NO: 64), respectively, are captured to the surface (each to a separate surface) and the test antibodies were added (each in a separate measurement) and the respective binding signals (Response Units (RU)) were determined. Reference signals (blank surface) were subtracted. If signals of nonbinding test antibodies were slightly below 0 the values were set as 0. Then the ratio of the respective binding signals (binding signal (RU) to human CSF-1R fragment delD4/binding signal (RU) to human CSF-1R Extracellular Domain (CSF-1R-ECD)) is determined. The antibodies according to the invention have a ratio of the binding signals (RU(delD4)/RU(CSF-1R-ECD) of 1:50 or lower, preferably of 1:100 or lower (the lower included end is 0 (e.g. if the RU is 0, then the ratio is 0:50 or 0:100)).

This means that such anti-CSF-1R antibodies according to the invention do not bind to the human CSF-1R fragment delD4 (like the anti-CCR5 antibody m<CCR5>Pz03.1C5 (deposited as DSM ACC 2683 on 18.08.2004 at DSMZ) and have binding signals for binding to the human CSF-1R fragment delD4 in the range of the anti-CCR5 antibody m<CCR5>Pz03.1C5, which are below 20 RU (Response Units), preferably below 10 RU in a Surface Plasmon Resonance (BIAcore) assay as shown in Example 4.

The term “binding to human CSF-1R fragment D1-D3” refers to a binding affinity determination by a Surface Plasmon Resonance assay (Biacore assay). The test antibody is captured to the surface and the human CSF-1R fragment D1-D3 (SEQ ID NO: 66) was added and the respective binding affinities were determined. The terms “not binding to human CSF-1R fragment D1-D3” or “which do not bind to human CSF-1R fragment D1-D3” denotes that in such an assay the detected signal was in the area of no more than 1.2 fold of background signal and therefore no significant binding could be detected and no binding affinity could be determined (see Example 10).

One embodiment of the invention is a screening method for selecting antibodies useful in a combination therapy according to the invention comprising the following steps:
a) measuring of the binding of anti-CSF-1R antibodies to human CSF-1R Extracellular Domain (CSF-1R-ECD) (SEQ ID NO: 64) by a Surface Plasmon Resonance assay (Biacore assay),
b) measuring of the binding of anti-CSF-1R antibodies to human CSF-1R fragment D1-D3 (SEQ ID NO: 66) (D1-D3),
c) selecting antibodies which specifically bind to human CSF-1R Extracellular Domain (CSF-1R-ECD) and which do not bind to human CSF-1R fragment D1-D3 (SEQ ID NO: 66) (D1-D3).

One embodiment of the invention is a screening method for selecting antibodies according to the invention comprising the following steps:
a) determining the binding signal (Response Units (RU)) of anti-CSF-1R antibodies to human CSF-1R fragment delD4 (SEQ ID NO: 65) and to human CSF-1R Extracellular Domain (CSF-1R-ECD) (SEQ ID NO: 64) by a Surface Plasmon Resonance assay (Biacore assay),
b) selecting antibodies with ratio of the binding signals (human CSF-1R fragment delD4/human CSF-1R Extracellular Domain (CSF-1R-ECD)) of 50:1 or lower.

In one embodiment the determination is performed at 25° C.

In one embodiment the screening method comprises as further steps the measuring of the binding of anti-CSF-1R antibodies to human CSF-1R fragment D1-D3 (SEQ ID NO: 66) (D1-D3) and the selecting of antibodies which show no binding to said fragment.

The term “epitope” denotes a protein determinant of human CSF-1R capable of specifically binding to an antibody. Epitopes usually consist of chemically active surface groupings of molecules such as amino acids or sugar side chains and usually epitopes have specific three dimensional structural characteristics, as well as specific charge characteristics. Conformational and nonconformational epitopes are distinguished in that the binding to the former but not the latter is lost in the presence of denaturing solvents. Preferably an antibody according to the invention binds specifically to native and to denatured CSF-1R.

The “variable domain” (variable domain of a light chain (V_{L}), variable domain of a heavy chain (V_{H})) as used herein denotes each of the pair of light and heavy chain domains which are involved directly in binding the antibody to the antigen. The variable light and heavy chain domains have the same general structure and each domain comprises four framework (FR) regions whose sequences are widely conserved, connected by three “hypervariable regions” (or complementary determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the antigen binding site. The antibody's heavy and light chain CDR3 regions play a particularly important role in the binding specificity/affinity of the antibodies according to the invention and therefore provide a further object of the invention.

The term “antigen-binding portion of an antibody” when used herein refer to the amino acid residues of an antibody which are responsible for antigen-binding. The antigen-binding portion of an antibody comprises amino acid residues from the “complementary determining regions” or “CDRs”. “Framework” or “FR” regions are those variable domain regions other than the hypervariable region residues as herein defined. Therefore, the light and heavy chain variable domains of an antibody comprise from N- to C-terminus the domains FR1, CDR1, FR2, CDR2, FR3, CDR3, and FR4. Especially, CDR3 of the heavy chain is the region which contributes most to antigen binding and defines the antibody's properties. CDR and FR regions are determined according to the standard definition of Kabat et al., Sequences of Proteins of Immunological Interest, 5th ed., Public Health Service, National Institutes of Health, Bethesda, Md. (1991) and/or those residues from a “hypervariable loop”.

The terms “nucleic acid” or “nucleic acid molecule”, as used herein, are intended to include DNA molecules and RNA molecules. A nucleic acid molecule may be single-stranded or double-stranded, but preferably is double-stranded DNA. The term “amino acid” as used within this application denotes the group of naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

In one embodiment the antibodies according to the invention inhibit CSF-1 binding to CSF-1R. In one embodiment with an IC50 of 200 ng/ml or lower, in one embodiment with an IC50 of 50 ng/ml or lower. The IC50 of inhibition of CSF-1 binding to CSF-1R can be determined as shown in Example 2.

In one embodiment the antibodies according to the invention inhibit CSF-1-induced CSF-1R phosphorylation (in NIH3T3-CSF-1R recombinant cells).

In one embodiment with an IC50 of 800 ng/ml or lower, in one embodiment with an IC50 of 600 ng/ml or lower, in one embodiment with an IC50 of 250 ng/ml or lower. The IC50 of CSF-1-induced CSF-1R phosphorylation can be determined as shown in Example 3.

In one embodiment the antibodies according to the invention inhibit the growth of recombinant NIH3T3 cells expressing human CSF-1R (SEQ ID No: 62). In one embodiment with an IC50 of 10 μg/ml or lower, in one embodiment with an IC50 of 5 μg/ml or lower, in one embodiment with an IC50 of 2 μg/ml or lower. In one embodiment with an IC30 of 10 μg/ml or lower, in one embodiment with an IC30 of 5 μg/ml or lower, in one embodiment with an IC30 of 2 μg/ml or lower. The IC50 value, the IC30 value or the % growth inhibition is determined as shown in Example 5.

In one embodiment the antibodies according to the invention inhibit the growth of recombinant NIH3T3 cells expressing human mutant CSF-1R L301S Y969F (SEQ ID No: 63). In one embodiment with an IC50 of 15 μg/ml or lower, in one embodiment with an IC50 of 10 μg/ml or lower. In one embodiment with an IC30 of 10 μg/ml or lower, in one embodiment with an IC50 of 5 μg/ml ng/ml or lower; in one embodiment with an IC50 of 2 μg/ml or lower. The IC50 value, the IC30 value or the % growth inhibition is determined as shown in Example 5. In one embodiment the antibodies according to the invention inhibit the growth of BeWo tumor cells (ATCC CCL-98) by 65% or more (at an antibody concentration of 10 μg/ml; and as compared to the absence of antibody). The % growth inhibition is determined as shown in Example 8. E.g. Mab 2F11 shows a growth inhibition of BeWo tumor cells of 70%.

In one embodiment the antibodies according to the invention inhibit (both) human and cynomolgous macrophage differentiation (which is indicated by the inhibition of the survival of human and cynomolgous monocytes as shown in Examples 7 and 8). In one embodiment the antibodies according to the invention inhibit the survival of human monocytes with an IC50 of 0.15 μg/ml or lower, in on embodiment with an IC50 of 0.10 μg/ml or lower. The inhibition of the survival of human monocytes is determined as shown in Example 7. In one embodiment the antibodies according to the invention inhibit the survival of cynomolgous monocytes by 80% or more, in one embodiment by 90% or more (at an antibody concentration of 5 μg/ml; and as compared to the absence of antibody). The inhibition of the survival of human monocytes is determined as shown in Example 8.

A further embodiment of the invention is a method for the production of an antibody against CSF-1R characterized in that the sequence of a nucleic acid encoding the heavy chain of a human IgG1 class antibody binding to human CSF-1R according to the invention said modified nucleic acid and the nucleic acid encoding the light chain of said antibody are inserted into an expression vector, said vector is inserted in a eukaryotic host cell, the encoded protein is expressed and recovered from the host cell or the supernatant.

The antibodies according to the invention are preferably produced by recombinant means. Therefore the antibody is preferably an isolated monoclonal antibody. Such recombinant methods are widely known in the state of the art and comprise protein expression in prokaryotic and eukaryotic cells with subsequent isolation of the antibody polypeptide and usually purification to a pharmaceutically acceptable purity. For the protein expression, nucleic acids encoding light and heavy chains or fragments thereof are inserted into expression vectors by standard methods. Expression is performed in appropriate prokaryotic or eukaryotic host cells like CHO cells, NS0 cells, SP2/0 cells, HEK293 cells, COS cells, yeast, or E. coli cells, and the antibody is recovered from the cells (supernatant or cells after lysis). Recombinant production of antibodies is well-known in the state of the art and described, for example, in the review articles of Makrides, S. C., Protein Expr. Purif. 17 (1999) 183-202; Geisse, S., et al., Protein Expr. Purif. 8 (1996) 271-282; Kaufman, R. J., Mol. Biotechnol. 16 (2000) 151-161; Werner, R. G., Drug Res. 48 (1998) 870-880.

The antibodies may be present in whole cells, in a cell lysate, or in a partially purified or substantially pure form. Purification is performed in order to eliminate other cellular components or other contaminants, e.g. other cellular nucleic acids or proteins, by standard techniques, including alkaline/SDS treatment, CsCl banding, column chromatography, agarose gel electrophoresis, and others well known in the art. See Ausubel, F., et al., ed. Current Protocols in Molecular Biology, Greene Publishing and Wiley Interscience, New York (1987).

Expression in NS0 cells is described by, e.g., Barnes, L. M., et al., Cytotechnology 32 (2000) 109-123; and Barnes, L. M., et al., Biotech. Bioeng. 73 (2001) 261-270. Transient expression is described by, e.g., Durocher, Y., et al., Nucl. Acids. Res. 30 (2002) E9. Cloning of variable domains is described by Orlandi, R., et al., Proc. Natl. Acad. Sci. USA 86 (1989) 3833-3837; Carter, P., et al., Proc. Natl. Acad. Sci. USA 89 (1992) 4285-4289; and Norderhaug, L., et al., J. Immunol. Methods 204 (1997) 77-87. A preferred transient expression system (HEK 293) is described by Schlaeger, E.-J., and Christensen, K., in Cytotechnology 30 (1999) 71-83 and by Schlaeger, E.-J., in J. Immunol. Methods 194 (1996) 191-199.

The control sequences that are suitable for prokaryotes, for example, include a promoter, optionally an operator sequence, and a ribosome binding site. Eukaryotic cells are known to utilize promoters, enhancers and polyadenylation signals. Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

The monoclonal antibodies are suitably separated from the culture medium by conventional immunoglobulin purification procedures such as, for example, protein A-Sepharose, hydroxylapatite chromatography, gel electrophoresis, dialysis, or affinity chromatography. DNA and RNA encoding the monoclonal antibodies are readily isolated and sequenced using conventional procedures. The hybridoma cells can serve as a source of such DNA and RNA. Once isolated, the DNA may be inserted into expression vectors, which are then transfected into host cells such as HEK 293 cells, CHO cells, or myeloma cells that do not otherwise produce immunoglobulin protein, to obtain the synthesis of recombinant monoclonal antibodies in the host cells.

As used herein, the expressions “cell”, “cell line”, and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included. The “Fc part” of an antibody is not involved directly in binding of an antibody to an antigen, but exhibit various effector functions. A “Fc part of an antibody” is a term well known to the skilled artisan and defined on the basis of papain cleavage of antibodies. Depending on the amino acid sequence of the constant region of their heavy chains, antibodies or immunoglobulins are divided in the classes: IgA, IgD, IgE, IgG and IgM, and several of these may be further divided into subclasses (isotypes), e.g. IgG1, IgG2, IgG3, and IgG4, IgA1, and IgA2. According to the heavy chain constant regions the different classes of immunoglobulins are called a, δ, ε, γ, and μ, respectively. The Fc part of an antibody is directly involved in ADCC (antibody-dependent cell-mediated cytotoxicity) and CDC (complement-dependent cytotoxicity) based on complement activation, C1q binding and Fc receptor binding. Complement activation (CDC) is initiated by binding of complement factor C1q to the Fc part of most IgG antibody subclasses. While the influence of an antibody on the complement system is dependent on certain conditions, binding to C1q is caused by defined binding sites in the Fc part. Such binding sites are known in the state of the art and described e.g. by Boackle, R. J., et al., Nature 282 (1979) 742-743; Lukas, T. J., et al., J. Immunol. 127 (1981) 2555-2560; Brunhouse, R., and Cebra, J. J., Mol. Immunol. 16 (1979) 907-917; Burton, D. R., et al., Nature 288 (1980) 338-344; Thommesen, J. E., et al., Mol. Immunol. 37 (2000) 995-1004; Idusogie, E. E., et al., J. Immunol.164 (2000) 4178-4184; Hezareh, M., et al., J. Virology 75 (2001) 12161-12168; Morgan, A., et al., Immunology 86 (1995) 319-324; EP 0 307 434. Such binding sites are e.g. L234, L235, D270, N297, E318, K320, K322, P331 and P329 (numbering according to EU index of Kabat, E. A., see below). Antibodies of subclass IgG1, IgG2 and IgG3 usually show complement activation and C1q and C3 binding, whereas IgG4 do not activate the complement system and do not bind C1q and C3.

In one embodiment the antibody according to the invention comprises a Fc part derived from human origin and preferably all other parts of the human constant regions. As used herein the term “Fc part derived from human origin” denotes a Fc part which is either a Fc part of a human antibody of the subclass IgG1, IgG2, IgG3 or IgG4, preferably a Fc part from human IgG1 subclass, a mutated Fc part from human IgG1 subclass (preferably with a mutation on L234A+L235A), a Fc part from human IgG4 subclass or a mutated Fc part from human IgG4 subclass (preferably with a mutation on S228P). Mostly preferred are the human heavy chain constant regions of SEQ ID NO: 58 (human IgG1 subclass), SEQ ID NO: 59 (human IgG1 subclass with mutations L234A and L235A), SEQ ID NO: 60 human IgG4 subclass), or SEQ ID NO: 61 (human IgG4 subclass with mutation S228P). Preferably the antibody according to the invention is of human IgG1 subclass or of human IgG4 subclass. In one embodiment the antibody according to the invention is of human IgG1 subclass. In one embodiment the antibody according to the invention is of human IgG4 subclass.

In one embodiment the antibody according to the invention is characterized in that the constant chains are of human origin. Such constant chains are well known in the state of the art and e.g. described by Kabat, E. A., (see e.g. Johnson, G. and Wu, T. T., Nucleic Acids Res. 28 (2000) 214-218). For example, a useful human heavy chain constant region comprises an amino acid sequence of SEQ ID NO: 58. For example, a useful human light chain constant region comprises an amino acid sequence of a kappa-light chain constant region of SEQ ID NO: 57.

Another aspect of the invention is the combination therapy with an antibody binding to human CSF-1R, characterized in that
a) the heavy chain variable domain is SEQ ID NO:7 and the light chain variable domain is SEQ ID NO:8,
b) the heavy chain variable domain is SEQ ID NO:15 and the light chain variable domain is SEQ ID NO:16;
   or a humanized version thereof.

Another aspect of the invention is the combination therapy with an antibody binding to human CSF-1R, characterized in that
a) the heavy chain variable domain is SEQ ID NO:7 and the light chain variable domain is SEQ ID NO:8,
b) the heavy chain variable domain is SEQ ID NO:15 and the light chain variable domain is SEQ ID NO:16;
c) the heavy chain variable domain is SEQ ID NO:75 and the light chain variable domain is SEQ ID NO:76;
d) the heavy chain variable domain is SEQ ID NO:83 and the light chain variable domain is SEQ ID NO:84;
   or a humanized version thereof.

Another aspect of the invention is the combination therapy with an antibody binding to human CSF-1R, characterized in that
the heavy chain variable domain is SEQ ID NO:7 and the light chain variable domain is SEQ ID NO:8, or a humanized version thereof.

Another aspect of the invention is the combination therapy with an antibody binding to human CSF-1R, characterized in that
a) the heavy chain variable domain is SEQ ID NO:23 and the light chain variable domain is SEQ ID NO:24, or
b) the heavy chain variable domain is SEQ ID NO:31 and the light chain variable domain is SEQ ID NO:32, or
c) the heavy chain variable domain is SEQ ID NO:39 and the light chain variable domain is SEQ ID NO:40, or
d) the heavy chain variable domain is SEQ ID NO:47 and the light chain variable domain is SEQ ID NO:48, or
e) the heavy chain variable domain is SEQ ID NO:55 and the light chain variable domain is SEQ ID NO:56.

Another aspect of the invention is the combination therapy with an antibody binding to human CSF-1R, characterized in that
a) the heavy chain variable domain is SEQ ID NO:23 and the light chain variable domain is SEQ ID NO:24, or
b) the heavy chain variable domain is SEQ ID NO:31 and the light chain variable domain is SEQ ID NO:32, or
c) the heavy chain variable domain is SEQ ID NO:39 and the light chain variable domain is SEQ ID NO:40, or
d) the heavy chain variable domain is SEQ ID NO:47 and the light chain variable domain is SEQ ID NO:48.

Another aspect of the invention is the combination therapy with an antibody binding to human CSF-1R, characterized in that
the heavy chain variable domain is SEQ ID NO:23 and the light chain variable domain is SEQ ID NO:24.

Another aspect of the invention is the combination therapy with an antibody binding to human CSF-1R, characterized in that
the heavy chain variable domain is SEQ ID NO:31 and the light chain variable domain is SEQ ID NO:32.

Another aspect of the invention is the combination therapy with an antibody binding to human CSF-1R, characterized in that
the heavy chain variable domain is SEQ ID NO:39 and the light chain variable domain is SEQ ID NO:40.

Another aspect of the invention is the combination therapy with an antibody binding to human CSF-1R, characterized in that
the heavy chain variable domain is SEQ ID NO:47 and the light chain variable domain is SEQ ID NO:48.

Another aspect of the invention is the combination therapy with an antibody binding to human CSF-1R, characterized in that
the heavy chain variable domain is SEQ ID NO:15 and the light chain variable domain is SEQ ID NO:16, or a humanized version thereof.

Another aspect of the invention is the combination therapy with an antibody binding to human CSF-1R, characterized in that
the heavy chain variable domain is SEQ ID NO:75 and the light chain variable domain is SEQ ID NO:76;
   or a humanized version thereof.

Another aspect of the invention is the combination therapy with an antibody binding to human CSF-1R, characterized in that
the heavy chain variable domain is SEQ ID NO:83 and the light chain variable domain is SEQ ID NO:84;
   or a humanized version thereof.

Another aspect of the invention is the combination therapy with an antibody binding to human CSF-1R, characterized in that
a) the heavy chain variable domain comprises a CDR3 region of SEQ ID NO:1, a CDR2 region of SEQ ID NO: 2, and a CDR1 region of SEQ ID NO:3, and the light chain variable domain comprises a CDR3 region of SEQ ID NO: 4, a CDR2 region of SEQ ID NO:5, and a CDR1 region of SEQ ID NO:6, or,
b) the heavy chain variable domain comprises a CDR3 region of SEQ ID NO: 9, a CDR2 region of SEQ ID NO: 10, and a CDR1 region of SEQ ID NO: 11, and the light chain variable domain comprises a CDR3 region of SEQ ID NO:12, a CDR2 region of SEQ ID NO: 13, and a CDR1 region of SEQ ID NO: 14, or
c) the heavy chain variable domain comprises a CDR3 region of SEQ ID NO: 17, a CDR2 region of SEQ ID NO: 18, and a CDR1 region of SEQ ID NO:19, and the light chain variable domain comprises a CDR3 region of SEQ ID NO: 20, a CDR2 region of SEQ ID NO:21, and a CDR1 region of SEQ ID NO:22, or
d) the heavy chain variable domain comprises a CDR3 region of SEQ ID NO: 25, a CDR2 region of SEQ ID NO: 26, and a CDR1 region of SEQ ID NO: 27, and the light chain variable domain comprises a CDR3 region of SEQ ID NO:28, a CDR2 region of SEQ ID NO: 29, and a CDR1 region of SEQ ID NO: 30, or
e) the heavy chain variable domain comprises a CDR3 region of SEQ ID NO: 33, a CDR2 region of SEQ ID NO: 34, and a CDR1 region of SEQ ID NO: 35, and the light chain variable domain comprises a CDR3 region of SEQ ID NO:36, a CDR2 region of SEQ ID NO: 37, and a CDR1 region of SEQ ID NO: 38, or
f) the heavy chain variable domain comprises a CDR3 region of SEQ ID NO:41, a CDR2 region of SEQ ID NO: 42, and a CDR1 region of SEQ ID NO:43, and the light chain variable domain comprises a CDR3 region of SEQ ID NO: 44, a CDR2 region of SEQ ID NO:45, and a CDR1 region of SEQ ID NO:46, or
g) the heavy chain variable domain comprises a CDR3 region of SEQ ID NO: 49, a CDR2 region of SEQ ID NO: 50, and a CDR1 region of SEQ ID NO: 51, and the light chain variable domain comprises a CDR3 region of SEQ ID NO:52, a CDR2 region of SEQ ID NO: 53, and a CDR1 region of SEQ ID NO: 54; or
h) the heavy chain variable domain comprises a CDR3 region of SEQ ID NO:69, a CDR2 region of SEQ ID NO: 70, and a CDR1 region of SEQ ID NO:71, and the light chain variable domain comprises a CDR3 region of SEQ ID NO: 72, a CDR2 region of SEQ ID NO:73, and a CDR1 region of SEQ ID NO:74, or
i) the heavy chain variable domain comprises a CDR3 region of SEQ ID NO: 77, a CDR2 region of SEQ ID NO: 78, and a CDR1 region of SEQ ID NO: 79, and the light chain variable domain comprises a CDR3 region of SEQ ID NO:80, a CDR2 region of SEQ ID NO: 81, and a CDR1 region of SEQ ID NO: 82.

Another aspect of the invention is the combination therapy with an antibody binding to human CSF-1R, characterized in that
a) the heavy chain variable domain comprises a CDR3 region of SEQ ID NO: 17, a CDR2 region of SEQ ID NO: 18, and a CDR1 region of SEQ ID NO:19, and the light chain variable domain comprises a CDR3 region of SEQ ID NO: 20, a CDR2 region of SEQ ID NO:21, and a CDR1 region of SEQ ID NO:22, or
b) the heavy chain variable domain comprises a CDR3 region of SEQ ID NO: 25, a CDR2 region of SEQ ID NO: 26, and a CDR1 region of SEQ ID NO: 27, and the light chain variable domain comprises a CDR3 region of SEQ ID NO:28, a CDR2 region of SEQ ID NO: 29, and a CDR1 region of SEQ ID NO: 30, or
c) the heavy chain variable domain comprises a CDR3 region of SEQ ID NO: 33, a CDR2 region of SEQ ID NO: 34, and a CDR1 region of SEQ ID NO: 35, and the light chain variable domain comprises a CDR3 region of SEQ ID NO:36, a CDR2 region of SEQ ID NO: 37, and a CDR1 region of SEQ ID NO: 38, or
d) the heavy chain variable domain comprises a CDR3 region of SEQ ID NO:41, a CDR2 region of SEQ ID NO: 42, and a CDR1 region of SEQ ID NO:43, and the light chain variable domain comprises a CDR3 region of SEQ ID NO: 44, a CDR2 region of SEQ ID NO:45, and a CDR1 region of SEQ ID NO:46, or
e) the heavy chain variable domain comprises a CDR3 region of SEQ ID NO: 49, a CDR2 region of SEQ ID NO: 50, and a CDR1 region of SEQ ID NO: 51, and the light chain variable domain comprises a CDR3 region of SEQ ID NO:52, a CDR2 region of SEQ ID NO: 53, and a CDR1 region of SEQ ID NO: 54.

Another aspect of the invention is the combination therapy with an antibody binding to human CSF-1R, characterized in that
a) the heavy chain variable domain comprises a CDR3 region of SEQ ID NO: 17, a CDR2 region of SEQ ID NO: 18, and a CDR1 region of SEQ ID NO:19, and the light chain variable domain comprises a CDR3 region of SEQ ID NO: 20, a CDR2 region of SEQ ID NO:21, and a CDR1 region of SEQ ID NO:22, or
b) the heavy chain variable domain comprises a CDR3 region of SEQ ID NO: 25, a CDR2 region of SEQ ID NO: 26, and a CDR1 region of SEQ ID NO: 27, and the light chain variable domain comprises a CDR3 region of SEQ ID NO:28, a CDR2 region of SEQ ID NO: 29, and a CDR1 region of SEQ ID NO: 30, or
c) the heavy chain variable domain comprises a CDR3 region of SEQ ID NO: 33, a CDR2 region of SEQ ID NO: 34, and a CDR1 region of SEQ ID NO: 35, and the light chain variable domain comprises a CDR3 region of SEQ ID NO:36, a CDR2 region of SEQ ID NO: 37, and a CDR1 region of SEQ ID NO: 38, or
d) the heavy chain variable domain comprises a CDR3 region of SEQ ID NO:41, a CDR2 region of SEQ ID NO: 42, and a CDR1 region of SEQ ID NO:43, and the light chain variable domain comprises a CDR3 region of SEQ ID NO: 44, a CDR2 region of SEQ ID NO:45, and a CDR1 region of SEQ ID NO:46.

Another aspect of the invention is the combination therapy with an antibody binding to human CSF-1R, characterized in that
the heavy chain variable domain comprises a CDR3 region of SEQ ID NO: 17, a CDR2 region of SEQ ID NO: 18, and a CDR1 region of SEQ ID NO:19, and the light chain variable domain comprises a CDR3 region of SEQ ID NO: 20, a CDR2 region of SEQ ID NO:21, and a CDR1 region of SEQ ID NO:22.

Another aspect of the invention is the combination therapy with an antibody binding to human CSF-1R, characterized in that
the heavy chain variable domain comprises a CDR3 region of SEQ ID NO: 25, a CDR2 region of SEQ ID NO: 26, and a CDR1 region of SEQ ID NO: 27, and the light chain variable domain comprises a CDR3 region of SEQ ID NO:28, a CDR2 region of SEQ ID NO: 29, and a CDR1 region of SEQ ID NO: 30.

Another aspect of the invention is the combination therapy with an antibody binding to human CSF-1R, characterized in that
the heavy chain variable domain comprises a CDR3 region of SEQ ID NO: 33, a CDR2 region of SEQ ID NO: 34, and a CDR1 region of SEQ ID NO: 35, and the light chain variable domain comprises a CDR3 region of SEQ ID NO:36, a CDR2 region of SEQ ID NO: 37, and a CDR1 region of SEQ ID NO: 38.

Another aspect of the invention is the combination therapy with an antibody binding to human CSF-1R, characterized in that
the heavy chain variable domain comprises a CDR3 region of SEQ ID NO:41, a CDR2 region of SEQ ID NO: 42, and a CDR1 region of SEQ ID NO:43, and the light chain variable domain comprises a CDR3 region of SEQ ID NO: 44, a CDR2 region of SEQ ID NO:45, and a CDR1 region of SEQ ID NO:46.

The invention comprises a method for the treatment of a patient in need of therapy, characterized by administering to the patient a therapeutically effective amount of an antibody according to the invention.

The invention comprises the use of an antibody according to the invention for the described therapy.

One preferred embodiment of the invention are the CSF-1R antibodies of the present invention for use in the treatment of “CSF-1R mediated diseases” or the CSF-1R antibodies of the present invention for use for the manufacture of a medicament in the treatment of “CSF-1R mediated diseases”, which can be described as follows:

There are 3 distinct mechanisms by which CSF-1R signaling is likely involved in tumor growth and metastasis. The first is that expression of CSF-ligand and receptor has been found in tumor cells originating in the female reproductive system (breast, ovarian, endometrium, cervical) (Scholl, S. M., et al., J. Natl. Cancer Inst. 86 (1994) 120-126; Kacinski, B. M., Mol. Reprod. Dev. 46 (1997) 71-74; Ngan, H. Y., et al., Eur. J. Cancer 35 (1999) 1546-1550; Kirma, N., et al., Cancer Res 67 (2007) 1918-1926) and the expression has been associated with breast cancer xenograft growth as well as poor prognosis in breast cancer patients. Two point mutations were seen in CSF-1R in about 10-20% of acute myelocytic leukemia, chronic myelocytic leukemia and myelodysplasia patients tested in one study, and one of the mutations was found to disrupt receptor turnover (Ridge, S. A., et al., Proc. Natl. Acad. Sci USA 87 (1990) 1377-1380). However the incidence of the mutations could not be confirmed in later studies (Abu-Duhier, F. M., et al., Br. J. Haematol. 120 (2003) 464-470). Mutations were also found in some cases of hepatocellular cancer (Yang, D. H., et al., Hepatobiliary Pancreat. Dis. Int. 3 (2004) 86-89) and idiopathic myelofibrosis (Abu-Duhier, F. M., et al., Br. J. Haematol. 120 (2003) 464-470). Recently, in the GDM-1 cell line derived from a patient with myelomonoblastic leukemia the Y571D mutation in CSF-1R was identified (Chase, A., et al., Leukemia 23 (2009) 358-364). Pigmented villonodular synovitis (PVNS) and Tenosynovial Giant cell tumors (TGCT) can occur as a result of a translocation that fuses the M-CSF gene to a collagen gene COL6A3 and results in overexpression of M-CSF (West, R. B., et al., Proc. Natl. Acad. Sci. USA 103 (2006) 690-695). A landscape effect is proposed to be responsible for the resulting tumor mass that consists of monocytic cells attracted by cells that express M-CSF. TGCTs are smaller tumors that can be relatively easily removed from fingers where they mostly occur. PVNS is more aggressive as it can recur in large joints and is not as easily controlled surgically.

The second mechanism is based on blocking signaling through M-CSF/CSF-1R at metastatic sites in bone which induces osteoclastogenesis, bone resorption and osteolytic bone lesions. Breast, multiple myeloma and lung cancers are examples of cancers that have been found to metastasize to the bone and cause osteolytic bone disease resulting in skeletal complications. M-CSF released by tumor cells and stroma induces the differentiation of hematopoietic myeloid monocyte progenitors to mature osteoclasts in collaboration with the receptor activator of nuclear factor kappa-B ligand-RANKL. During this process, M-CSF acts as a permissive factor by giving the survival signal to osteoclasts (Tanaka, S., et al., J. Clin. Invest. 91 (1993) 257-263). Inhibition of CSF-1R activity during osteoclast differentiation and maturation with an anti-CSF-1R antibody is likely to prevent unbalanced activity of osteoclasts that cause osteolytic disease and the associated skeletal related events in metastatic disease. Whereas breast, lung cancer and multiple myeloma typically result in osteolytic lesions, metastasis to the bone in prostate cancer initially has an osteoblastic appearance in which increased bone forming activity results in ‘woven bone’ which is different from typical lamellar structure of normal bone. During disease progression bone lesions display a significant osteolytic component as well as high serum levels of bone resorption and suggests that anti-resorptive therapy may be useful. Bisphosphonates have been shown to inhibit the formation of osteolytic lesions and reduced the number of skeletal-related events only in men with hormone-refractory metastatic prostate cancer but at this point their effect on osteoblastic lesions is controversial and bisphosphonates have not been beneficial in preventing bone metastasis or hormone responsive prostate cancer to date. The effect of anti-resorptive agents in mixed osteolytic/osteoblastic prostate cancer is still being studied in the clinic (Choueiri, M. B., et al., Cancer Metastasis Rev. 25 (2006) 601-609; Vessella, R. L. and Corey, E., Clin. Cancer Res. 12 (20 Pt 2) (2006) 6285s-6290s).

The third mechanism is based on the recent observation that tumor associated macrophages (TAM) found in solid tumors of the breast, prostate, ovarian and cervical cancers correlated with poor prognosis (Bingle, L., et al., J. Pathol. 196 (2002) 254-265; Pollard, J. W., Nat. Rev. Cancer 4 (2004) 71-78). Macrophages are recruited to the tumor by M-CSF and other chemokines. The macrophages can then contribute to tumor progression through the secretion of angiogenic factors, proteases and other growth factors and cytokines and may be blocked by inhibition of CSF-1R signaling. Recently it was shown by Zins et al (Zins, K., et al., Cancer Res. 67 (2007) 1038-1045) that expression of siRNA of Tumor necrosis factor alpha (TNF alpha), M-CSF or the combination of both would reduce tumor growth in a mouse xenograft model between 34% and 50% after intratumoral injection of the respective siRNA. SiRNA targeting the TNF alpha secreted by the human SW620 cells reduced mouse M-CSF levels and led to reduction of macrophages in the tumor. In addition treatment of MCF7 tumor xenografts with an antigen binding fragment directed against M-CSF did result in 40% tumor growth inhibition, reversed the resistance to chemotherapeutics and improved survival of the mice when given in combination with chemotherapeutics (Paulus, P., et al., Cancer Res. 66 (2006) 4349-4356).

TAMs are only one example of an emerging link between chronic inflammation and cancer. There is additional evidence for a link between inflammation and cancer as many chronic diseases are associated with an increased risk of cancer, cancers arise at sites of chronic inflammation, chemical mediators of inflammation are found in many cancers; deletion of the cellular or chemical mediators of inflammation inhibits development of experimental cancers and long-term use of anti-inflammatory agents reduce the risk of some cancers. A link to cancer exists for a number of inflammatory conditions among-those H. pylori induced gastritis for gastric cancer, Schistosomiasis for bladder cancer, HHVX for Kaposi's sarcoma, endometriosis for ovarian cancer and prostatitis for prostate cancer (Balkwill, F., et al., Cancer Cell 7 (2005) 211-217). Macrophages are key cells in chronic inflammation and respond differentially to their microenvironment. There are two types of macrophages that are considered extremes in a continuum of functional states: M1 macrophages are involved in Type 1 reactions. These reactions involve the activation by microbial products and consequent killing of pathogenic microorganisms that result in reactive oxygen intermediates. On the other end of the extreme are M2 macrophages involved in Type 2 reactions that promote cell proliferation, tune inflammation and adaptive immunity and promote tissue remodeling, angiogenesis and repair (Mantovani, A., et al., Trends Immunol. 25 (2004) 677-686). Chronic inflammation resulting in established neoplasia is usually associated with M2 macrophages. A pivotal cytokine that mediates inflammatory reactions is TNF alpha that true to its name can stimulate anti-tumor immunity and hemorrhagic necrosis at high doses but has also recently been found to be expressed by tumor cells and acting as a tumor promoter (Zins, K., et al., Cancer Res. 67 (2007) 1038-1045; Balkwill, F., Cancer Metastasis Rev. 25 (2006) 409-416). The specific role of macrophages with respect to the tumor still needs to be better understood including the potential spatial and temporal dependence on their function and the relevance to specific tumor types.

Thus one embodiment of the invention are the CSF-1R antibodies of the present invention for use in the treatment of cancer. The term “cancer” as used herein may be, for example, lung cancer, non small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, lymphoma, lymphocytic leukemia, including refractory versions of any of the above cancers, or a combination of one or more of the above cancers. Preferably such cancer is a breast cancer, ovarian cancer, cervical cancer, lung cancer or prostate cancer. Preferably such cancers are further characterized by CSF-1 or CSF-1R expression or overexpression. One further embodiment the invention are the CSF-1R antibodies of the present invention for use in the simultaneous treatment of primary tumors and new metastases.

Thus another embodiment of the invention are the CSF-1R antibodies of the present invention for use in the treatment of periodontitis, histiocytosis X, osteoporosis, Paget's disease of bone (PDB), bone loss due to cancer therapy, periprosthetic osteolysis, glucocorticoid-induced osteoporosis, rheumatoid arthritis, psiratic arthritis, osteoarthritis, inflammatory arthridities, and inflammation. Rabello, D., et al., Biochem. Biophys. Res. Commun. 347 (2006) 791-796 has demonstrated that SNPs in the CSF1 gene exhibited a positive association with aggressive periodontitis: an inflammatory disease of the periodontal tissues that causes tooth loss due to resorption of the alveolar bone.

Histiocytosis X (also called Langerhans cell histiocytosis, LCH) is a proliferative disease of Langerhans dendritic cells that appear to differentiate into osteoclasts in bone and extra osseous LCH lesions. Langerhans cells are derived from circulating monocytes. Increased levels of M-CSF that have been measured in sera and lesions where found to correlate with disease severity (da Costa, C. E., et al., J. Exp. Med. 201 (2005) 687-693). The disease occurs primarily in a pediatric patient population and has to be treated with chemotherapy when the disease becomes systemic or is recurrent.

The pathophysiology of osteoporosis is mediated by loss of bone forming osteoblasts and increased osteoclast dependent bone resorption. Supporting data has been described by Cenci et al showing that an anti-M-CSF antibody injection preserves bone density and inhibits bone resorption in ovariectomized mice (Cenci, S., et al., J. Clin. Invest. 105 (2000) 1279-1287). Recently a potential link between postmenopausal bone loss due to estrogen deficiency was identified and found that the presence of TNF alpha producing T-cell affected bone metabolism (Roggia, C., et al., Minerva Med. 95 (2004) 125-132). A possible mechanism could be the induction of M-CSF by TNF alpha in vivo. An important role for M-CSF in TNF-alpha-induced osteoclastogenesis was confirmed by the effect of an antibody directed against M-CSF that blocked the TNF alpha induced osteolysis in mice and thereby making inhibitors of CSF-1R signaling potential targets for inflammatory arthritis (Kitaura, H., et al., J. Clin. Invest. 115 (2005) 3418-3427). Paget's disease of bone (PDB) is the second most common bone metabolism disorder after osteoporosis in which focal abnormalities of increased bone turnover lead to complications such as bone pain, deformity, pathological fractures and deafness. Mutations in four genes have been identified that regulate normal osteoclast function and predispose individuals to PDB and related disorders: insertion mutations in TNFRSF11A, which encodes receptor activator of nuclear factor (NF) kappaB (RANK)-a critical regulator of osteoclast function, inactivating mutations of TNFRSF11B which encodes osteoprotegerin (a decoy receptor for RANK ligand), mutations of the sequestosome 1 gene (SQSTM1), which encodes an important scaffold protein in the NFkappaB pathway and mutations in the valosin-containing protein (VCP) gene. This gene encodes VCP, which has a role in targeting the inhibitor of NFkappaB for degradation by the proteasome (Daroszewska, A. and Ralston, S. H., Nat. Clin. Pract. Rheumatol. 2 (2006) 270-277). Targeted CSF-1R inhibitors provide an opportunity to block the deregulation of the RANKL signaling indirectly and add an additional treatment option to the currently used bisphosphonates.

Cancer therapy induced bone loss especially in breast and prostate cancer patients is an additional indication where a targeted CSF-1R inhibitor could prevent bone loss (Lester, J. E., et al., Br. J. Cancer 94 (2006) 30-35). With the improved prognosis for early breast cancer the long-term consequences of the adjuvant therapies become more important as some of the therapies including chemotherapy, irradiation, aromatase inhibitors and ovary ablation affect bone metabolism by decreasing the bone mineral density, resulting in increased risk for osteoporosis and associated fractures (Lester, J. E., et al., Br. J. Cancer 94 (2006) 30-35). The equivalent to adjuvant aromatase inhibitor therapy in breast cancer is androgen ablation therapy in prostate cancer which leads to loss of bone mineral density and significantly increases the risk of osteoporosis-related fractures (Stoch, S. A., et al., J. Clin. Endocrinol. Metab. 86 (2001) 2787-2791).

Targeted inhibition of CSF-1R signaling is likely to be beneficial in other indications as well when targeted cell types include osteoclasts and macrophages e.g. treatment of specific complications in response to joint replacement as a consequence of rheumatoid arthritis. Implant failure due to periprosthetic bone loss and consequent loosing of prostheses is a major complication of joint replacement and requires repeated surgery with high socioeconomic burdens for the individual patient and the health-care system. To date, there is no approved drug therapy to prevent or inhibit periprosthetic osteolysis (Drees, P., et al., Nat. Clin. Pract. Rheumatol. 3 (2007) 165-171).

Glucocorticoid-induced osteoporosis (GIOP) is another indication in which a CSF-1R inhibitor could prevent bone loss after longterm glucocorticocosteroid use that is given as a result of various conditions among those chronic obstructive pulmonary disease, asthma and rheumatoid arthritis (Guzman-Clark, J. R., et al., Arthritis Rheum. 57 (2007) 140-146; Feldstein, A. C., et al., Osteoporos. Int. 16 (2005) 2168-2174).

Rheumatoid arthritis, psoriatic arthritis and inflammatory arthridities are in itself potential indications for CSF-1R signaling inhibitors in that they consist of a macrophage component and to a varying degree bone destruction (Ritchlin, C. T., et al., J. Clin. Invest. 111 (2003) 821-831). Osteoarthritis and rheumatoid arthritis are inflammatory autoimmune disease caused by the accumulation of macrophages in the connective tissue and infiltration of macrophages into the synovial fluid, which is at least partially mediated by M-CSF. Campbell, I., K., et al., J. Leukoc. Biol. 68 (2000) 144-150, demonstrated that M-CSF is produced by human-joint tissue cells (chondrocytes, synovial fibroblasts) in vitro and is found in synovial fluid of patients with rheumatoid arthritis, suggesting that it contributes to the synovial tissue proliferation and macrophage infiltration which is associated with the pathogenesis of the disease. Inhibition of CSF-1R signaling is likely to control the number of macrophages in the joint and alleviate the pain from the associated bone destruction. In order to minimize adverse effects and to further understand the impact of the CSF-1R signaling in these indications, one method is to specifically inhibit CSF-1R without targeting a myriad other kinases, such as Raf kinase. Recent literature reports correlate increased circulating M-CSF with poor prognosis and atherosclerotic progression in chronic coronary artery disease (Saitoh, T., et al., J. Am. Coll. Cardiol. 35 (2000) 655-665; Ikonomidis, I., et al., Eur. Heart. J. 26 (2005) p. 1618-1624); M-CSF influences the atherosclerotic process by aiding the formation of foam cells (macrophages with ingested oxidized LDL) that express CSF-1R and represent the initial plaque (Murayama, T., et al., Circulation 99 (1999) 1740-1746).

Expression and signaling of M-CSF and CSF-1R is found in activated microglia. Microglia, which are resident macrophages of the central nervous system, can be activated by various insults, including infection and traumatic injury. M-CSF is considered a key regulator of inflammatory responses in the brain and M-CSF levels increase in HIV-1, encephalitis, Alzheimer's disease (AD) and brain tumors. Microgliosis as a consequence of autocrine signaling by M-CSF/CSF-1R results in induction of inflammatory cytokines and nitric oxides being released as demonstrated by e.g. using an experimental neuronal damage model (Hao, A. J., et al., Neuroscience 112 (2002) 889-900; Murphy, G. M., Jr., et al., J. Biol. Chem. 273 (1998) 20967-20971). Microglia that have increased expression of CSF-1R are found to surround plaques in AD and in the amyloid precursor protein V717F transgenic mouse model of AD (Murphy, G. M., Jr., et al., Am. J. Pathol. 157 (2000) 895-904). On the other hand op/op mice with fewer microglia in the brain resulted in fibrilar deposition of A-beta and neuronal loss compared to normal control suggesting that microglia do have a neuroprotective function in the development of AD lacking in the op/op mice (Kaku, M., et al., Brain Res. Brain Res. Protoc. 12 (2003) 104-108).

Expression and signaling of M-CSF and CSF-1R is associated with inflammatory bowel disease (IBD) (WO 2005/046657). The term “inflammatory bowel disease” refers to serious, chronic disorders of the intestinal tract characterized by chronic inflammation at various sites in the gastrointestinal tract, and specifically includes ulcerative colitis (UC) and Crohn's disease.
The invention comprises the combination therapy with an antibody binding to human CSF-1R being characterized by the above mentioned epitope binding properties or alternatively by the above mentioned amino acid sequences and amino acid sequence fragments for the treatment of cancer.
The invention comprises the combination therapy with an antibody binding to human CSF-1R being characterized by the above mentioned epitope binding properties or alternatively by the above mentioned amino acid sequences and amino acid sequence fragments for the treatment of bone loss.
The invention comprises the combination therapy with an antibody binding to human CSF-1R being characterized by the above mentioned epitope binding properties or alternatively by the above mentioned amino acid sequences and amino acid sequence fragments for the prevention or treatment of metastasis.
The invention comprises the combination therapy with an antibody binding to human CSF-1R being characterized by the above mentioned epitope binding properties or alternatively by the above mentioned amino acid sequences and amino acid sequence fragments for treatment of inflammatory diseases.
The invention comprises the use of an antibody characterized in comprising the antibody binding to human CSF-1R being characterized by the above mentioned epitope binding properties or alternatively by the above mentioned amino acid sequences and amino acid sequence fragments for the combination treatment of cancer as described herein or alternatively for the manufacture of a medicament for the combination treatment of cancer as described herein.
The invention comprises the use of an antibody characterized in comprising the antibody binding to human CSF-1R being characterized by the above mentioned epitope binding properties or alternatively by the above mentioned amino acid sequences and amino acid sequence fragments for the combination treatment as described herein of bone loss or alternatively for the manufacture of a medicament for the combination treatment as described herein of bone loss.
The invention comprises the use of an antibody characterized in comprising the antibody binding to human CSF-1R being characterized by the above mentioned epitope binding properties or alternatively by the above mentioned amino acid sequences and amino acid sequence fragments for the prevention or treatment of metastasis with the combination as described herein or alternatively for the manufacture of a medicament for the prevention or treatment of metastasis with the combination as described herein.
The invention comprises the use of an antibody characterized in comprising the antibody binding to human CSF-1R being characterized by the above mentioned epitope binding properties or alternatively by the above mentioned amino acid sequences and amino acid sequence fragments for combination treatment of inflammatory diseases as described herein or alternatively for the manufacture of a medicament for the combination treatment of inflammatory diseases as described herein.

The antibodies according to the invention are preferably produced by recombinant means. Such methods are widely known in the state of the art and comprise protein expression in prokaryotic and eukaryotic cells with subsequent isolation of the antibody polypeptide and usually purification to a pharmaceutically acceptable purity. For the protein expression nucleic acids encoding light and heavy chains or fragments thereof are inserted into expression vectors by standard methods. Expression is performed in appropriate prokaryotic or eukaryotic host cells, such as CHO cells, NS0 cells, SP2/0 cells, HEK293 cells, COS cells, yeast, or E. coli cells, and the antibody is recovered from the cells (from the supernatant or after cells lysis).

Recombinant production of antibodies is well-known in the state of the art and described, for example, in the review articles of Makrides, S. C., Protein Expr. Purif. 17 (1999) 183-202; Geisse, S., et al., Protein Expr. Purif. 8 (1996) 271-282; Kaufman, R. J., Mol. Biotechnol. 16 (2000) 151-161; Werner, R. G., Drug Res. 48 (1998) 870-880.

The antibodies may be present in whole cells, in a cell lysate, or in a partially purified, or substantially pure form. Purification is performed in order to eliminate other cellular components or other contaminants, e.g. other cellular nucleic acids or proteins, by standard techniques, including alkaline/SDS treatment, CsCl banding, column chromatography, agarose gel electrophoresis, and others well known in the art. See Ausubel, F., et al., ed. Current Protocols in Molecular Biology, Greene Publishing and Wiley Interscience, New York (1987).

Expression in NS0 cells is described by, e.g., Barnes, L. M., et al., Cytotechnology 32 (2000) 109-123; Barnes, L. M., et al., Biotech. Bioeng. 73 (2001) 261-270. Transient expression is described by, e.g., Durocher, Y., et al., Nucl. Acids. Res. 30 (2002) E9. Cloning of variable domains is described by Orlandi, R., et al., Proc. Natl. Acad. Sci. USA 86 (1989) 3833-3837; Carter, P., et al., Proc. Natl. Acad. Sci. USA 89 (1992) 4285-4289; Norderhaug, L., et al., J. Immunol. Methods 204 (1997) 77-87. A preferred transient expression system (HEK 293) is described by Schlaeger, E.-J. and Christensen, K., in Cytotechnology 30 (1999) 71-83, and by Schlaeger, E.-J., in J. Immunol. Methods 194 (1996) 191-199.

Nucleic acid molecules encoding amino acid sequence variants of anti-CSF-1R antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of humanized anti-CSF-1R antibody.

The heavy and light chain variable domains according to the invention are combined with sequences of promoter, translation initiation, constant region, 3′ untranslated region, polyadenylation, and transcription termination to form expression vector constructs. The heavy and light chain expression constructs can be combined into a single vector, co-transfected, serially transfected, or separately transfected into host cells which are then fused to form a single host cell expressing both chains.

In another aspect, the present invention provides a composition, e.g. a pharmaceutical composition, containing one or a combination of monoclonal antibodies, or the antigen-binding portion thereof, of the present invention, formulated together with a pharmaceutically acceptable carrier.

As used herein, “pharmaceutically acceptable carrier” includes any and all solvents, dispersion media, coatings, antibacterial and antifungal agents, isotonic and absorption/resorption delaying agents, and the like that are physiologically compatible. Preferably, the carrier is suitable for injection or infusion.

A composition of the present invention can be administered by a variety of methods known in the art. As will be appreciated by the skilled artisan, the route and/or mode of administration will vary depending upon the desired results. Pharmaceutically acceptable carriers include sterile aqueous solutions or dispersions and sterile powders for the preparation of sterile injectable solutions or dispersion. The use of such media and agents for pharmaceutically active substances is known in the art. In addition to water, the carrier can be, for example, an isotonic buffered saline solution.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient (effective amount). The selected dosage level will depend upon a variety of pharmacokinetic factors including the activity of the particular compositions of the present invention employed, or the ester, salt or amide thereof, the route of administration, the time of administration, the rate of excretion of the particular compound being employed, other drugs, compounds and/or materials used in combination with the particular compositions employed, the age, sex, weight, condition, general health and prior medical history of the patient being treated, and like factors well known in the medical arts.

The term “a method of treating” or its equivalent, when applied to, for example, cancer refers to a procedure or course of action that is designed to reduce or eliminate the number of cancer cells in a patient, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of a patient, is nevertheless deemed to induce an overall beneficial course of action.

The terms “administered in combination with” or “co-administration”, “co-administering” refer to the administration of the anti-CSF-1R, and the chemotherapeutic agent, radiotherapy and/or cancer immunotherapy e.g. as separate formulations/applications (or as one single formulation/application). The co-administration can be simultaneous or sequential in either order, wherein preferably there is a time period while both (or all) active agents simultaneously exert their biological activities. Said antibody and said further agent are co-administered either simultaneously or sequentially (e.g. intravenous (i.v.) through a continuous infusion. When both therapeutic agents are co-administered sequentially the dose is administered either on the same day in two separate administrations, or one of the agents is administered on day 1 and the second is co-administered on day 2 to day 7, preferably on day 2 to 4. Thus in one embodiment the term “sequentially” means within 7 days after the dose of the first component, preferably within 4 days after the dose of the first component; and the term “simultaneously” means at the same time. The terms “co-administration” with respect to the maintenance doses of anti-CSF-1R antibody mean that the maintenance doses can be either co-administered simultaneously, if the treatment cycle is appropriate for both drugs, e.g. every week. Or the further agent is e.g. administered e.g. every first to third day and said antibody is administered every week. Or the maintenance doses are co-administered sequentially, either within one or within several days.

It is self-evident that the antibodies are administered to the patient in a “therapeutically effective amount” (or simply “effective amount”) which is the amount of the respective compound or combination that will elicit the biological or medical response of a tissue, system, animal or human that is being sought by the researcher, veterinarian, medical doctor or other clinician.

The amount of co-administration and the timing of co-administration will depend on the type (species, gender, age, weight, etc.) and condition of the patient being treated and the severity of the disease or condition being treated. Said anti-CSF-1R antibody and further agent are suitably co-administered to the patient at one time or over a series of treatments e.g. on the same day or on the day after.

Depending on the type and severity of the disease, about 0.1 mg/kg to 50 mg/kg (e.g. 0.1-20 mg/kg) of said anti-CSF-1R antibody; is an initial candidate dosage for co-administration of both drugs to the patient The invention comprises the use of the antibodies according to the invention for the treatment of a patient suffering from cancer, especially from colon, lung or pancreas cancer.

The invention comprises also a method for the treatment of a patient suffering from such disease.

The invention further provides a method for the manufacture of a pharmaceutical composition comprising an effective amount of an antibody according to the invention together with a pharmaceutically acceptable carrier and the use of the antibody according to the invention for such a method.

The invention further provides the use of an antibody according to the invention in an effective amount for the manufacture of a pharmaceutical agent, preferably together with a pharmaceutically acceptable carrier, for the treatment of a patient suffering from cancer.

The invention also provides the use of an antibody according to the invention in an effective amount for the manufacture of a pharmaceutical agent, preferably together with a pharmaceutically acceptable carrier, for the treatment of a patient suffering from cancer.

The following examples, sequence listing and figures are provided to aid the understanding of the present invention, the true scope of which is set forth in the appended claims. It is understood that modifications can be made in the procedures set forth without departing from the spirit of the invention.

### Description of the Sequences

SEQ ID NO: 1 heavy chain CDR3, Mab 2F11
SEQ ID NO: 2 heavy chain CDR2, Mab 2F11
SEQ ID NO: 3 heavy chain CDR1, Mab 2F11
SEQ ID NO: 4 light chain CDR3, Mab 2F11
SEQ ID NO: 5 light chain CDR2, Mab 2F11
SEQ ID NO: 6 light chain CDR1, Mab 2F11
SEQ ID NO: 7 heavy chain variable domain, Mab 2F11
SEQ ID NO: 8 light chain variable domain, Mab 2F11
SEQ ID NO: 9 heavy chain CDR3, Mab 2E10
SEQ ID NO: 10 heavy chain CDR2, Mab 2E10
SEQ ID NO: 11 heavy chain CDR1, Mab 2E10
SEQ ID NO: 12 light chain CDR3, Mab 2E10
SEQ ID NO: 13 light chain CDR2, Mab 2E10
SEQ ID NO: 14 light chain CDR1, Mab 2E10
SEQ ID NO: 15 heavy chain variable domain, Mab 2E10
SEQ ID NO: 16 light chain variable domain, Mab 2E10
SEQ ID NO: 17 heavy chain CDR3, hMab 2F11-c11
SEQ ID NO: 18 heavy chain CDR2, hMab 2F11-c11
SEQ ID NO: 19 heavy chain CDR1, hMab 2F11-c11
SEQ ID NO: 20 light chain CDR3, hMab 2F11-c11
SEQ ID NO: 21 light chain CDR2, hMab 2F11-c11
SEQ ID NO: 22 light chain CDR1, hMab 2F11-c11
SEQ ID NO: 23 heavy chain variable domain, hMab 2F11-c11
SEQ ID NO: 24 light chain variable domain, hMab 2F11-c11
SEQ ID NO: 25 heavy chain CDR3, hMab 2F11-d8
SEQ ID NO: 26 heavy chain CDR2, hMab 2F11-d8
SEQ ID NO: 27 heavy chain CDR1, hMab 2F11-d8
SEQ ID NO: 28 light chain CDR3, hMab 2F11-d8
SEQ ID NO: 29 light chain CDR2, hMab 2F11-d8
SEQ ID NO: 30 light chain CDR1, hMab 2F11-d8
SEQ ID NO: 31 heavy chain variable domain, hMab 2F11-d8
SEQ ID NO: 32 light chain variable domain, hMab 2F11-d8
SEQ ID NO: 33 heavy chain CDR3, hMab 2F11-e7
SEQ ID NO: 34 heavy chain CDR2, hMab 2F11-e7
SEQ ID NO: 35 heavy chain CDR1, hMab 2F11-e7
SEQ ID NO: 36 light chain CDR3, hMab 2F11-e7
SEQ ID NO: 37 light chain CDR2, hMab 2F11-e7
SEQ ID NO: 38 light chain CDR1, hMab 2F11-e7
SEQ ID NO: 39 heavy chain variable domain, hMab 2F11-e7
SEQ ID NO: 40 light chain variable domain, hMab 2F11-e7
SEQ ID NO: 41 heavy chain CDR3, hMab 2F11-f12
SEQ ID NO: 42 heavy chain CDR2, hMab 2F11-f12
SEQ ID NO: 43 heavy chain CDR1, hMab 2F11-f12
SEQ ID NO: 44 light chain CDR3, hMab 2F11-f12
SEQ ID NO: 45 light chain CDR2, hMab 2F11-f12
SEQ ID NO: 46 light chain CDR1, hMab 2F11-f12
SEQ ID NO: 47 heavy chain variable domain, hMab 2F11-f12
SEQ ID NO: 48 light chain variable domain, hMab 2F11-f12
SEQ ID NO: 49 heavy chain CDR3, hMab 2F11-g1
SEQ ID NO: 50 heavy chain CDR2, hMab 2F11-g1
SEQ ID NO: 51 heavy chain CDR1, hMab 2F11-g1
SEQ ID NO: 52 light chain CDR3, hMab 2F11-g1
SEQ ID NO: 53 light chain CDR2, hMab 2F11-g1
SEQ ID NO: 54 light chain CDR1, hMab 2F11-g1
SEQ ID NO: 55 heavy chain variable domain, hMab 2F11-g1
SEQ ID NO: 56 light chain variable domain, hMab 2F11-g1
SEQ ID NO: 57 human kappa light chain constant region
SEQ ID NO: 58 human heavy chain constant region derived from IgG1
SEQ ID NO: 59 human heavy chain constant region derived from IgG1 mutated on L234A and L235A
SEQ ID NO: 60 human heavy chain constant region derived from IgG4
SEQ ID NO: 61 human heavy chain constant region derived from IgG4 mutated on S228P
SEQ ID NO: 62 human wildtype CSF-1R (wt CSF-1R)
SEQ ID NO: 63 human mutant CSF-1R L301S Y969F
SEQ ID NO: 64 human CSF-1R Extracellular Domain (domains D1-D5)
SEQ ID NO: 65 human CSF-1R fragment delD4
SEQ ID NO: 66 human CSF-1R fragment domains D1-D3
SEQ ID NO: 67 signal peptide
SEQ ID NO: 68 Primer
SEQ ID NO: 69 heavy chain CDR3, Mab 1G10
SEQ ID NO: 70 heavy chain CDR2, Mab 1G10
SEQ ID NO: 71 heavy chain CDR1, Mab 1G10
SEQ ID NO: 72 light chain CDR3, Mab 1G10
SEQ ID NO: 73 light chain CDR2, Mab 1G10
SEQ ID NO: 74 light chain CDR1, Mab 1G10
SEQ ID NO: 75 heavy chain variable domain, Mab 1G10
SEQ ID NO: 76 light chain variable domain, Mab 1G10
SEQ ID NO: 77 heavy chain CDR3, Mab 2H7
SEQ ID NO: 78 heavy chain CDR2, Mab 2H7
SEQ ID NO: 79 heavy chain CDR1, Mab 2H7
SEQ ID NO: 80 light chain CDR3, Mab 2H7
SEQ ID NO: 81 light chain CDR2, Mab 2H7
SEQ ID NO: 82 light chain CDR1, Mab 2H7
SEQ ID NO: 83 heavy chain variable domain, Mab 2H7
SEQ ID NO: 84 light chain variable domain, Mab 2H7
SEQ ID NO: 85 human CSF-1R fragment domains D4-D5
SEQ ID NO: 86 human CSF-1
SEQ ID NO: 87 human IL-34
SEQ ID NO: 88 heavy chain variable domain of CP-870,893 (antibody 21.4.1 of U.S. Pat. No. 7,338,660) SEQ ID NO: 89 light chain variable domain of CP-870,893 (antibody 21.4.1 of U.S. Pat. No. 7,338,660)
SEQ ID NO: 90 humanized S2C6 heavy chain variable domain variant
SEQ ID NO: 91 humanized S2C6 light chain variable domain variant

In the following. some embodiments of the invention are described:
1. A) An antibody binding to human CSF-1R, characterized in binding to the (dimerization) domains D4 to D5 (SEQ ID No: 85) of the extracellular domain of human CSF-1R for use in
   a) the inhibition of cell proliferation in CSF-1R ligand-dependent and/or CSF-1R ligand-independent CSF-1R expressing tumor cells;
   b) the inhibition of cell proliferation of tumors with CSF-1R ligand-dependent and/or CSF-1R ligand-independent CSF-1R expressing macrophage infiltrate;
   c) the inhibition of cell survival (in CSF-1R ligand-dependent and/or CSF-1R ligand-independent) CSF-1R expressing monocytes and macrophages; and/or
   d) the inhibition of cell differentiation (in CSF-1R ligand-dependent and/or CSF-1R ligand-independent) CSF-1R expressing monocytes into macrophages;
   wherein the anti-CSF-1R antibody is administered in combination with a chemotherapeutic agent, radiation, and/or cancer immunotherapy;
or B) An antibody binding to human CSF-1R, characterized in binding to the domains D4 to D5 (SEQ ID No: 85) of the extracellular domain of human CSF-1R for use in
   the treatment of a patient having a CSF-1R expressing tumor or having a tumor with CSF-1R expressing macrophage infiltrate, wherein the tumor is characterized by an increase of CSF-1R ligand.
   wherein the anti-CSF-1R antibody is administered in combination with a chemotherapeutic agent, radiation and/or cancer immunotherapy.
2. A) Use of an antibody binding to human CSF-1R, characterized in binding to the (dimerization) domains D4 to D5 (SEQ ID No: 85) of the extracellular domain of human CSF-1R for use in the manufacture of a medicament for
   a) the inhibition of cell proliferation in CSF-1R ligand-dependent and/or CSF-1R ligand-independent CSF-1R expressing tumor cells;
   b) the inhibition of cell proliferation of tumors with CSF-1R ligand-dependent and/or CSF-1R ligand-independent CSF-1R expressing macrophage infiltrate;
   c) the inhibition of cell survival (in CSF-1R ligand-dependent and/or CSF-1R ligand-independent) CSF-1R expressing monocytes and macrophages; and/or
   d) the inhibition of cell differentiation (in CSF-1R ligand-dependent and/or CSF-1R ligand-independent) CSF-1R expressing monocytes into macrophages;
   wherein the anti-CSF-1R antibody is administered in combination with a chemotherapeutic agent, radiation, and/or cancer immunotherapy;
or B) Use of an antibody binding to human CSF-1R, characterized in binding to the domains D4 to D5 (SEQ ID No: 85) of the extracellular domain of human CSF-1R for use in the manufacture of a medicament for the the treatment of a patient having a CSF-1R expressing tumor or having a tumor with CSF-1R expressing macrophage infiltrate, wherein the tumor is characterized by an increase of CSF-1R ligand
   wherein the anti-CSF-1R antibody is administered in combination with a chemotherapeutic agent, radiation and/or cancer immunotherapy.
3. The antibody or use according to embodiments 1 or 2, wherein the chemotherapeutic agent is selected from the group consisting of taxanes (paclitaxel (Taxol), docetaxel (Taxotere), modified paclitaxel (Abraxane and Opaxio)), doxorubicin, modified doxorubicin (Caelyx or Doxil)), sunitinib (Sutent), sorafenib (Nexavar), and other multikinase inhibitors, oxaliplatin, cisplatin and carboplatin, etoposide, gemcitabine, and vinblastine.
4. The antibody or use according to embodiments 1 or 2, wherein the cancer immunotherapy is selected from the group of:
   a) T cell engaging agents selected from agonistic antibodies which bind to human OX40, TO GITR, TO CD27, OR TO 4-1BB, and T-cell bispecific antibodies (e.g. T cell-engaging BiTE™ antibodies CD3-CD19, CD3-EpCam, CD3-EGFR), IL-2 (Proleukin), Interferon (IFN) alpha, antagonizing antibodies which bind to human CTLA-4 (e.g. ipilimumab), to PD-1, to PD-L1, to TIM-3, to BTLA, to VISTA, to LAG-3, or to CD25,
   b) targeting immunosuppression: antibodies or small molecules targeting STATS or NFkB signaling, blocking IL-6, IL-17, IL-23, TNFa function,
   c) cancer vaccines/enhance dendritic cell function: OncoVex (oncolytic virus secreting GM-CSF), an agonistic CD40 antibody, Toll-like receptor (TLR) ligands, TLR agonists, recombinant fusion protein encoding MAGE-A3, PROSTVAC; or
   d) adoptive cell transfer: GVAX (prostate cancer cell line expressing GM-CSF), dendritic cell vaccine, adoptive T cell therapy, adoptive CAR T cell therapy.
5. The antibody or use according to embodiment 4, wherein the cancer immunotherapy is an agonistic CD40 antibody (in one embodiment the agonistic CD40 antibody is CP-870,893 or SGN-40).
6. The antibody or use according to embodiments 1 or 2, wherein the chemotherapeutic agent is selected from the group of taxanes (docetaxel or paclitaxel or a modified paclitaxel (Abraxane or Opaxio)), doxorubicin, capecitabine and/or bevacizumab for the treatment of breast cancer.
7. The antibody or use according to embodiments 1 or 2, wherein the chemotherapeutic agent is selected from the group of carboplatin, oxaliplatin, cisplatin, paclitaxel, doxorubicin (or modified doxorubicin (Caelyx or Doxil)), or topotecan (Hycamtin) for the treatment of ovarian cancer.
8. The antibody or use according to embodiments 1 or 2, wherein the chemotherapeutic agent is selected from the group of a multi-kinase inhibitor (sunitinib (Sutent), sorafenib (Nexavar) or motesanib diphosphate (AMG 706) and/or doxorubicin for treatment of kidney cancer.
9. The antibody according to embodiments 1 or 2, wherein the chemotherapeutic agent is selected from the group of oxaliplatin, cisplatin and/or radiation for the treatment of squamous cell carcinoma.
10. The antibody or use according to embodiments 1 or 2, wherein the chemotherapeutic agent is selected from the group of taxol and/or carboplatin for the treatment of lung cancer.
11. The antibody according any one of the preceding embodiments, wherein the antibody is characterized in that the antibody does not bind to human CSF-1R fragment delD4 (SEQ ID NO: 65).
12. The antibody or use according any one of the preceding embodiments, wherein the antibody is characterized in that
   the antibody binds to human CSF-1R fragment delD4 (SEQ ID NO: 65) and to human CSF-1R Extracellular Domain (SEQ ID NO: 64) with a ratio of 1:50 or lower.
13. The antibody according any one of the preceding embodiments, characterized in that
   a) the heavy chain variable domain is SEQ ID NO:7 and the light chain variable domain is SEQ ID NO:8,
   b) the heavy chain variable domain is SEQ ID NO:15 and the light chain variable domain is SEQ ID NO:16;
   c) the heavy chain variable domain is SEQ ID NO:75 and the light chain variable domain is SEQ ID NO:76;
   d) the heavy chain variable domain is SEQ ID NO:83 and the light chain variable domain is SEQ ID NO:84;
   or a humanized version thereof.
14. The antibody according any one of the preceding embodiments, characterized in that
   a) the heavy chain variable domain is SEQ ID NO:23 and the light chain variable domain is SEQ ID NO:24, or
   b) the heavy chain variable domain is SEQ ID NO:31 and the light chain variable domain is SEQ ID NO:32, or
   c) the heavy chain variable domain is SEQ ID NO:39 and the light chain variable domain is SEQ ID NO:40, or
   d) the heavy chain variable domain is SEQ ID NO:47 and the light chain variable domain is SEQ ID NO:48, or
   e) the heavy chain variable domain is SEQ ID NO:55 and the light chain variable domain is SEQ ID NO:56.
15. The antibody according any one of the preceding embodiments, characterized in that
   a) the heavy chain variable domain comprises a CDR3 region of SEQ ID NO: 1, a CDR2 region of SEQ ID NO: 2, and a CDR1 region of SEQ ID NO:3, and the light chain variable domain comprises a CDR3 region of SEQ ID NO: 4, a CDR2 region of SEQ ID NO:5, and a CDR1 region of SEQ ID NO:6, or
   b) the heavy chain variable domain comprises a CDR3 region of SEQ ID NO: 9, a CDR2 region of SEQ ID NO: 10, and a CDR1 region of SEQ ID NO: 11, and the light chain variable domain comprises a CDR3 region of SEQ ID NO:12, a CDR2 region of SEQ ID NO: 13, and a CDR1 region of SEQ ID NO: 14, or
   c) the heavy chain variable domain comprises a CDR3 region of SEQ ID NO: 17, a CDR2 region of SEQ ID NO: 18, and a CDR1 region of SEQ ID NO:19, and the light chain variable domain comprises a CDR3 region of SEQ ID NO: 20, a CDR2 region of SEQ ID NO:21, and a CDR1 region of SEQ ID NO:22, or
   d) the heavy chain variable domain comprises a CDR3 region of SEQ ID NO: 25, a CDR2 region of SEQ ID NO: 26, and a CDR1 region of SEQ ID NO: 27, and the light chain variable domain comprises a CDR3 region of SEQ ID NO:28, a CDR2 region of SEQ ID NO: 29, and a CDR1 region of SEQ ID NO: 30, or
   e) the heavy chain variable domain comprises a CDR3 region of SEQ ID NO: 33, a CDR2 region of SEQ ID NO: 34, and a CDR1 region of SEQ ID NO: 35, and the light chain variable domain comprises a CDR3 region of SEQ ID NO:36, a CDR2 region of SEQ ID NO: 37, and a CDR1 region of SEQ ID NO: 38, or
   f) the heavy chain variable domain comprises a CDR3 region of SEQ ID NO:41, a CDR2 region of SEQ ID NO: 42, and a CDR1 region of SEQ ID NO:43, and the light chain variable domain comprises a CDR3 region of SEQ ID NO: 44, a CDR2 region of SEQ ID NO:45, and a CDR1 region of SEQ ID NO:46, or
   g) the heavy chain variable domain comprises a CDR3 region of SEQ ID NO: 49, a CDR2 region of SEQ ID NO: 50, and a CDR1 region of SEQ ID NO: 51, and the light chain variable domain comprises a CDR3 region of SEQ ID NO:52, a CDR2 region of SEQ ID NO: 53, and a CDR1 region of SEQ ID NO: 54; or
   h) the heavy chain variable domain comprises a CDR3 region of SEQ ID NO:69, a CDR2 region of SEQ ID NO: 70, and a CDR1 region of SEQ ID NO:71, and the light chain variable domain comprises a CDR3 region of SEQ ID NO: 72, a CDR2 region of SEQ ID NO:73, and a CDR1 region of SEQ ID NO:74, or
   i) the heavy chain variable domain comprises a CDR3 region of SEQ ID NO: 77, a CDR2 region of SEQ ID NO: 78, and a CDR1 region of SEQ ID NO: 79, and the light chain variable domain comprises a CDR3 region of SEQ ID NO:80, a CDR2 region of SEQ ID NO: 81, and a CDR1 region of SEQ ID NO: 82.
16. The antibody according any one of the preceding embodiments, characterized in that said antibody is of human IgG1 subclass or is of human IgG4 subclass.
17. The antibody or use according any one of the preceding embodiments for use in a method of treatment of cancer, of bone loss, of metastasis, of inflammatory diseases, or for use in the prevention of metastasis.
18. A) A method for
   a) the inhibition of cell proliferation in CSF-1R ligand-dependent and/or CSF-1R ligand-independent CSF-1R expressing tumor cells;
   b) the inhibition of cell proliferation of tumors with CSF-1R ligand-dependent and/or CSF-1R ligand-independent CSF-1R expressing macrophage infiltrate;
   c) the inhibition of cell survival (in CSF-1R ligand-dependent and/or CSF-1R ligand-independent) CSF-1R expressing monocytes and macrophages; and/or
   d) the inhibition of cell differentiation (in CSF-1R ligand-dependent and/or CSF-1R ligand-independent) CSF-1R expressing monocytes into macrophages;
   wherein an antibody binding to human CSF-1R, characterized in binding to the (dimerization) domains D4 to D5 (SEQ ID No: 85) of the extracellular domain of human CSF-1R is administered in combination with a chemotherapeutic agent, radiation, and/or cancer immunotherapy;
or B) A method of treatment of a patient having a CSF-1R expressing tumor or having a tumor with CSF-1R expressing macrophage infiltrate, wherein the tumor is characterized by an increase of CSF-1R ligand wherein an antibody binding to human CSF-1R, characterized in binding to the domains D4 to D5 (SEQ ID No: 85) of the extracellular domain of human CSF-1R for use in is administered in combination with a chemotherapeutic agent, radiation and/or cancer immunotherapy.
19. An antibody binding to human CSF-1R, for use in
   the treatment of a patient having a CSF-1R expressing tumor or having a tumor with CSF-1R expressing macrophage infiltrate, wherein the tumor is characterized by an increase of CSF-1R ligand
   wherein the anti-CSF-1R antibody is administered in combination with a cancer immunotherapy.
   wherein the cancer immunotherapy is selected from the group of:
a) T cell engaging agents selected from agonistic antibodies which bind to human OX40, to GITR, to CD27, or to 4-1BB, and T-cell bispecific antibodies (e.g. T cell-engaging BiTE™ antibodies CD3-CD19, CD3-EpCam, CD3-EGFR), IL-2 (Proleukin), Interferon (IFN) alpha, antagonizing antibodies which bind to human CTLA-4 (e.g. ipilimumab), to PD-1, to PD-L1, to TIM-3, to BTLA, to VISTA, to LAG-3, or to CD25,
b) targeting immunosuppression: antibodies or small molecules targeting STATS or NFkB signaling, blocking IL-6, IL-17, IL-23, TNFa function,
c) cancer vaccines/enhance dendritic cell function: OncoVex (oncolytic virus secreting GM-CSF), an agonistic CD40 antibody, Toll-like receptor (TLR) ligands, TLR agonists, recombinant fusion protein encoding MAGE-A3, PROSTVAC; or
d) adoptive cell transfer: GVAX (prostate cancer cell line expressing GM-CSF), dendritic cell vaccine, adoptive T cell therapy, adoptive CAR T cell therapy.
20. The antibody according to embodiment 19
   wherein the cancer immunotherapy is selected from the group of: cancer vaccines/enhance dendritic cell function: OncoVex (oncolytic virus secreting GM-CSF), an agonistic CD40 antibody, Toll-like receptor (TLR) ligands, TLR agonists, recombinant fusion protein encoding MAGE-A3, PROSTVAC.
21. The antibody or use according to embodiment 19, wherein the cancer immunotherapy is an agonistic CD40 antibody (in one embodiment the agonistic CD40 antibody is CP-870,893 or SGN-40).
22. A method for determining whether a subject having a cancer is a candidate for an anti-CSF-1R antibody-based cancer treatment regimen, the method comprising:
   ex vivo or in vitro determining in vitro the level of one or more of the following markers:
   CSF-1R, CD68/CD163, CD68/MHC class II, CD31 (microvessel density), and Ki67 and other markers like e g immuninfiltrates;
   in a sample of the subject, wherein the sample is selected from the group consisting of tissue, blood, serum, plasma, tumor cells and circulating tumor cells; and
   wherein a change in the level of one or more of CSF-1R, CD68/CD163, CD68/MHC class II, CD31 (microvessel density) and Ki67 and other markers like e.g. immuninfiltrates (e.g. T cells (e.g. CD4- and/or CD8-T cells), as compared with to the corresponding level in an individual not suffering from cancer, is indicative that the subject is a candidate for the anti-CSF-1 R antibody-based cancer treatment regimen.
23. The method of embodiment 22, wherein the antibody used in said regimen is an antibody according to any of the preceding embodiments.
24. The method of embodiments 21 or 22 wherein in this method the change in the level of CSF-1R, CD68/CD163, CD68/MHC class II, CD31 (microvessel density) and Ki67 and other markers like e.g. immuninfiltrates (e.g. T cells (e.g. CD4- and/or CD8-T cells), as compared to the level in an individual not suffering from cancer is an increase in the level of one or more of these markers.
25. A method for determining whether a subject having a cancer is a candidate for an anti-CSF-1R antibody-based cancer treatment regimen, the method comprising:
   ex vivo or in vitro determining in vitro the level of one or more of the following markers:
   CSF-1, Trap5b, sCD163, IL-34;
   in a sample of the subject, wherein the sample is selected from the group consisting of tissue, blood, serum, plasma, tumor cells and circulating tumor cells; and
   wherein a change in the level of one or more of CSF-1, Trap5b, sCD163, IL-34, as compared with to the corresponding level in an individual not suffering from cancer, is indicative that the subject is a candidate for the anti-CSF-1 R antibody-based cancer treatment regimen.
26. The method of embodiment 25, wherein the antibody used in said regimen is an antibody according to any of the preceding embodiments.
27. The method of embodiments 25 or 26 wherein in this method the change in the level of CSF-1, Trap5b, sCD163, IL-34, as compared to the level in an individual not suffering from cancer is an increase in the level of one or more of these markers.
28. The method of any of embodiments 25 to 27 wherein in this method ex vivo or in vitro the level and change of the level of sCD163 is determined.
29. A method for determining whether a subject having a cancer is a candidate for an anti-CSF-1R antibody-based cancer treatment regimen, the method comprising:
   ex vivo or in vitro determining in vitro the level of one or more of the following markers:
   IFNγ, TNFα, IL-1β, IL-4, IL-6, IL-8, IL-10, IL-13, GM-CSF, VEGF, MCP-1, CCL18, CCL22, MIP-1, Galectin 3, IL1Ra, TGF alpha;
   in a sample of the subject, wherein the sample is selected from the group consisting of tissue, blood, serum, plasma, tumor cells and circulating tumor cells; and
   wherein a change in the level of one or more of IFNγ, TNFα, IL-1β, IL-4, IL-6, IL-8, IL-10, IL-13, GM-CSF, VEGF, MCP-1, CCL18, CCL22, MIP-1, Galectin 3, IL1Ra, TGF alpha, as compared with to the corresponding level in an individual not suffering from cancer, is indicative that the subject is a candidate for the anti-CSF-1 R antibody-based cancer treatment regimen.
30. The method of embodiment 29, wherein the antibody used in said regimen is an antibody according to any of the preceding embodiments.
31. The method of embodiments 29 or 30 wherein in this method the change in the level of IFNγ, TNFα, IL-1β, IL-4, IL-6, IL-8, IL-10, IL-13, GM-CSF, VEGF, MCP-1, CCL18, CCL22, MIP-1, Galectin 3, IL1Ra, TGF alpha, as compared to the level in an individual not suffering from cancer is an increase in the level of one or more of these markers.
32. An antibody binding to human CSF-1R for use in the treatment of cancer wherein the antibody is administered in combination with a bispecific ANG-2-VEGF antibody.
33. An antibody binding to human CSF-1R for use in the treatment of cancer wherein the anti-CSF-1R antibody is administered in combination with an agonistic CD40 antibody.
34. The antibody binding to human CSF-1R according to embodiment 33, wherein the anti-CSF-1R antibody comprises (a) a heavy chain variable domain amino acid sequence of SEQ ID NO:39 and (b) a light chain variable domain amino acid sequence of SEQ ID NO:40; and
   wherein the agonistic CD40 antibody is CP-870,893 (antibody 21.4.1 of U.S. Pat. No. 7,338,660).
35. The antibody binding to human CSF-1R according to embodiment 33,
   i) wherein the anti-CSF-1R antibody comprises (a) a heavy chain variable domain amino acid sequence of SEQ ID NO:39 and (b) a light chain variable domain amino acid sequence of SEQ ID NO:40; and
   ii) wherein the agonistic CD40 antibody comprises (a) a heavy chain variable domain amino acid sequence of SEQ ID NO: 88 and (b) a light chain variable domain amino acid sequence of SEQ ID NO: 89.
36. The antibody binding to human CSF-1R according to embodiment 33, wherein the anti-CSF-1R antibody comprises (a) a heavy chain variable domain amino acid sequence of SEQ ID NO:39 and (b) a light chain variable domain amino acid sequence of SEQ ID NO:40; and wherein the agonistic CD40 antibody is dacetuzumab.
37. The antibody binding to human CSF-1R according to embodiment 33,
   i) wherein the anti-CSF-1R antibody comprises (a) a heavy chain variable domain amino acid sequence of SEQ ID NO:39 and (b) a light chain variable domain amino acid sequence of SEQ ID NO:40; and
   ii) wherein the agonistic CD40 antibody comprises (a) a heavy chain variable domain amino acid sequence of SEQ ID NO: 90 and (b) a light chain variable domain amino acid sequence of SEQ ID NO: 91.
38. The antibody binding to human CSF-1R according to embodiment 33, wherein the agonistic CD40 antibody is
   i) CP-870,893;
   ii) a) comprises (a) a heavy chain variable domain amino acid sequence of SEQ ID NO: 88) and (b) a light chain variable domain amino acid sequence of SEQ ID NO: 89;
   iii) is dacetuzumab; or
   iv) comprises (a) a heavy chain variable domain amino acid sequence of SEQ ID NO: 90 and (b) a light chain variable domain amino acid sequence of SEQ ID NO: 91.
39. A method of treating cancer/or use of an antibody binding to human CSF-1R for the manufacture of a medicament for the treatment of cancer wherein the anti-CSF-1R antibody is administered in combination with an agonistic CD40 antibody.
40. The method/use according to embodiment 39, wherein the anti-CSF-1R antibody comprises (a) a heavy chain variable domain amino acid sequence of SEQ ID NO:39 and (b) a light chain variable domain amino acid sequence of SEQ ID NO:40; and
   wherein the agonistic CD40 antibody is CP-870,893 (antibody 21.4.1 of U.S. Pat. No. 7,338,660).
41. The method/use according to embodiment 39,
   i) wherein the anti-CSF-1R antibody comprises (a) a heavy chain variable domain amino acid sequence of SEQ ID NO:39 and (b) a light chain variable domain amino acid sequence of SEQ ID NO:40; and
   ii) wherein the agonistic CD40 antibody comprises (a) a heavy chain variable domain amino acid sequence of SEQ ID NO: 88 and (b) a light chain variable domain amino acid sequence of SEQ ID NO: 89.
42. The method/use according to embodiment 39, wherein the anti-CSF-1R antibody comprises (a) a heavy chain variable domain amino acid sequence of SEQ ID NO:39 and (b) a light chain variable domain amino acid sequence of SEQ ID NO:40; and
   wherein the agonistic CD40 antibody is dacetuzumab.
43. The method/use according to embodiment 39,
   i) wherein the anti-CSF-1R antibody comprises (a) a heavy chain variable domain amino acid sequence of SEQ ID NO:39 and (b) a light chain variable domain amino acid sequence of SEQ ID NO:40; and
   ii) wherein the agonistic CD40 antibody comprises (a) a heavy chain variable domain amino acid sequence of SEQ ID NO: 90 and (b) a light chain variable domain amino acid sequence of SEQ ID NO: 91.
44. The method/use according to embodiment 39,
   wherein the agonistic CD40 antibody is
   i) CP-870,893;
   ii) a) comprises (a) a heavy chain variable domain amino acid sequence of SEQ ID NO: 88) and (b) a light chain variable domain amino acid sequence of SEQ ID NO: 89;
   iii) is dacetuzumab; or
   iv) comprises (a) a heavy chain variable domain amino acid sequence of SEQ ID NO: 90 and (b) a light chain variable domain amino acid sequence of SEQ ID NO: 91. The following examples, sequence listing and figures are provided to aid the understanding of the present invention, the true scope of which is set forth in the appended claims. It is understood that modifications can be made in the procedures set forth without departing from the spirit of the invention.

### EXAMPLES

### Example 1

### Generation of a Hybridoma Cell Line Producing Anti-CSF-1R Antibodies Immunization Procedure of NMRI Mice

NMRI mice were immunized with an expression vector pDisplay™ (Invitrogen, USA) encoding the extracellular domain of huCSF-1R by utilizing electroporation. Every mouse was 4 times immunized with 100 μg DNA. When serum titers of anti-huCSF-1R were found to be sufficient, mice were additionally boosted once with 50 μg of a 1:1 mixture huCSF-1R ECD/huCSF-1R ECDhuFc chimera in 200 μl PBS intravenously (i.v.) 4 and 3 days before fusion.

### Antigen Specific ELISA

Anti-CSF-1R titers in sera of immunized mice were determined by antigen specific ELISA.

0.3 μg/ml huCSF-1R-huFc chimera (soluble extracellular domain) was captured on a streptavidin plate (MaxiSorb; MicroCoat, DE, Cat. No. 11974998/MC1099) with 0.1 mg/ml biotinylated anti Fcγ (Jackson ImmunoResearch., Cat. No. 109-066-098) and horse radish peroxidase (HRP)-conjugated F(ab′)₂ anti-mouse IgG (GE Healthcare, UK, Cat. No. NA9310V) diluted 1/800 in PBS/0.05% Tween20/0.5% BSA was added. Sera from all taps were diluted 1/40 in PBS/0.05% Tween20/0.5% BSA and serially diluted up to 1/1638400. Diluted sera were added to the wells. Pre-tap serum was used as negative control. A dilution series of mouse anti-human CSF-1R Mab3291 (R&D Systems, UK) from 500 ng/ml to 0.25 ng/ml was used as positive control. All components were incubated together for 1.5 hours, Wells were washed 6 times with PBST (PBS/0.2% Tween20) and assays were developed with freshly prepared ABTS® solution (1 mg/ml) (ABTS: 2,2′-azino bis (3-ethylbenzthiazoline-6-sulfonic acid) for 10 minutes at RT. Absorbance was measured at 405 nm.

### Hybridoma Generation

The mouse lymphocytes can be isolated and fused with a mouse myeloma cell line using PEG based standard protocols to generate hybridomas. The resulting hybridomas are then screened for the production of antigen-specific antibodies. For example, single cell suspensions of splenic derived lymphocytes from immunized mice are fused to Ag8 non-secreting mouse myeloma cells P3X63Ag8.653 (ATCC, CRL-1580) with 50% PEG. Cells are plated at approximately 10⁴ in flat bottom 96 well micro titer plate, followed by about two weeks incubation in selective medium. Individual wells are then screened by ELISA for human anti-CSF-1R monoclonal IgM and IgG antibodies. Once extensive hybridoma growth occurs, the antibody secreting hybridomas are replated, screened again, and if still positive for human IgG, anti-CSF-1R monoclonal antibodies, can be subcloned by FACS. The stable subclones are then cultured in vitro to produce antibody in tissue culture medium for characterization. Antibodies according to the invention could be selected using the determination of the binding of anti-CSF-1R antibodies to human CSF-1R fragment delD4 and to human CSF-1R Extracellular Domain (CSF-1R-ECD) as described in Example 4, as well as the determination of growth inhibition of NIH3T3 cells transfected with wildtype CSF-1R (ligand dependent signalling) or mutant CSF-1R L301S Y969F (ligand independent signalling) under treatment with anti-CSF-1R monoclonal antibodies as described in Example 5.

### Culture of Hybridomas

Generated muMAb hybridomas were cultured in RPMI 1640 (PAN—Catalogue No. (Cat. No.) PO4-17500) supplemented with 2 mM L-glutamine (GIBCO—Cat. No. 35050-038), 1 mM Na-Pyruvat (GIBCO—Cat. No. 11360-039), 1×NEAA (GIBCO—Cat. No. 11140-035), 10% FCS (PAA—Cat. No. A15-649), 1× Pen Strep (Roche—Cat. No. 1074440), 1× Nutridoma CS (Roche—Cat. No. 1363743), 50 μM Mercaptoethanol (GIBCO—Cat. No. 31350-010) and 50 U/ml IL 6 mouse (Roche-Cat. No. 1 444 581) at 37° C. and 5% CO₂. Some of the resulting mouse antibodies have been humanized (e.g. Mab 2F11) and been expressed recombinantly.

### Example 2

Inhibition of CSF-1 binding to CSF-1R (ELISA)

By setting-up this assay to first allow for anti-CSF-1R antibody binding to the CSF-1R-ECD followed by detection of ligand not bound to the receptor both—ligand displacing antibodies and dimerization inhibitor anti-CSF-1R antibodies—can be tested. The test was performed on 384 well microtiter plates (MicroCoat, DE, Cat. No. 464718) at RT. After each incubation step plates were washed 3 times with PBST.

At the beginning, plates were coated with 0.5 mg/ml goat F(ab′)2 biotinylated anti Fcγ (Jackson ImmunoResearch., Cat. No. 109-006-170) for 1 hour (h). Thereafter the wells were blocked with PBS supplemented with 0.2% Tween®-20 and 2% BSA (Roche Diagnostics GmbH, DE) for 0.5 h. 75 ng/ml of huCSF-1R-huFc chimera (which forms the dimeric soluble extracellular domain of huCSF-1R) was immobilized to plate for 1 h. Then dilutions of purified antibodies in PBS/0.05% Tween20/0.5% BSA were incubated for 1 h. After adding a mixture of 3 ng/ml hu CSF-1 (active 149 aa fragment of human CSF-1 (aa 33-181 of SEQ ID NO: 86); Biomol, DE, Cat. No. 60530), 50 ng/ml biotinylated anti CSF-1 clone BAF216 (R&D Systems,UK) and 1:5000 diluted streptavidin HRP (Roche Diagnostics GmbH, DE, Cat. No. 11089153001) for 1 h the plates were washed 6 times with PBST. Anti CSF-1R SC 2-4A5 (Santa Cruz Biotechnology, US), which inhibits the ligand-receptor interaction, was used as positive control. Plates were developed with freshly prepared BM Blue® POD substrate solution (BM Blue®: 3,3′-5,5′-Tetramethylbenzidine, Roche Diagnostics GmbH, DE, Cat. No. 11484281001) for 30 minutes at RT. Absorbance was measured at 370 nm. A decrease of absorbance is found, if the anti-CSF-1R antibody causes a release of CSF-1 from the dimeric complex. All anti-CSF-1R antibodies showed significant inhibition of the CSF-1 interaction with CSF-1R (see Table 1). Anti CSF-1R SC 2-4A5 (Santa Cruz Biotechnology, US see also Sherr, C. J. et al., Blood 73 (1989) 1786-1793), which inhibits the ligand-receptor interaction, was used as reference control.

TABLE 1
Calculated IC50 values for the inhibition
of the CSF-1/CSF-1R interaction

| | IC50 CSF-1/CSF-1R |
|---|---|
| CSF-1R Mab | Inhibition [ng/ml] |
| Mab 2F11 | 19.3 |
| Mab 2E10 | 20.6 |
| Mab 2H7 | 18.2 |
| Mab 1G10 | 11.8 |
| SC-2-4A5 | 35.2 |

### Example 3

### Inhibition of CSF-1-Induced CSF-1R Phosphorylation in NIH3T3-CSF-1R Recombinant Cells

4.5×10³ NIH 3T3 cells, retrovirally infected with an expression vector for full-length CSF-1R, were cultured in DMEM (PAA Cat. No. E15-011), 2 mM L-glutamine (Sigma, Cat. No. G7513, 2 mM Sodium pyruvate, 1× nonessential aminoacids, 10% FKS (PAA, Cat. No. A15-649) and 100 μg/ml PenStrep (Sigma, Cat. No. P4333 [10 mg/ml]) until they reached confluency. Thereafter cells were washed with serum-free DMEM media (PAA Cat. No. E15-011) supplemented with sodium selenite [5 ng/ml] (Sigma, Cat. No. S9133), transferrin [10 μg/ml] (Sigma, Cat. No. T8158), BSA [400 μg/ml] (Roche Diagnostics GmbH, Cat. No. 10735078), 4 mM L-glutamine (Sigma, Cat. No. G7513), 2 mM sodium pyruvate (Gibco, Cat. No. 11360), 1× nonessential aminoacids (Gibco, Cat: 11140-035), 2-mercaptoethanol [0.05 mM] (Merck, Cat. No. M7522), 100 μg/ml and PenStrep (Sigma, Cat. No. P4333) and incubated in 30 μl of the same medium for 16 hours to allow for receptor up-regulation. 10 μl of diluted anti-CSR-1R antibodies were added to the cells for 1.5 h. Then cells were stimulated with 10 μl of 100 ng/ml hu CSF-1 (active 149 aa fragment of human CSF-1 (aa 33-181 of SEQ ID NO: 86); Biomol, DE, Cat. No. 60530) for 5 min. After the incubation, supernatant was removed, cells were washed twice with 80 μl of ice-cold PBS and 50 μl of freshly prepared ice-cold lysis buffer (150 mM NaCl/20 mM Tris pH 7.5/1 mM EDTA/1 mM EGTA/1% Triton X-100/1 protease inhibitor tablet (Roche Diagnostics GmbH Cat. No. 1 836 170) per 10 ml buffer/10 μl/ml phosphatase inhibitor cocktail 1 (Sigma Cat. No. P-2850, 100× Stock)/10 μl/ml protease inhibitor 1 (Sigma Cat. No. P-5726, 100× Stock)/10 μl/ml 1 M NaF) was added. After 30 minutes on ice the plates were shaken vigorously on a plateshaker for 3 minutes and then centrifuged 10 minutes at 2200 rpm (Heraeus Megafuge 10).

The presence of phosphorylated and total CSF-1 receptor in the cell lysate was analyzed with Elisa. For detection of the phosphorylated receptor the kit from R&D Systems (Cat. No. DYC3268-2) was used according to the instructions of the supplier. For detection of total CSF-1R 10 μl of the lysate was immobilized on plate by use of the capture antibody contained in the kit. Thereafter 1:750 diluted biotinylated anti CSF-1R antibody BAF329 (R&D Systems) and 1:1000 diluted streptavidin-HRP conjugate was added. After 60 minutes plates were developed with freshly prepared ABTS® solution and the absorbance was detected. Data were calculated as % of positive control without antibody and the ratio value phospho/total receptor expressed. The negative control was defined without addition of M-CSF-1. Anti CSF-1R SC 2-4A5 (Santa Cruz Biotechnology, US, see also Sherr, C. J. et al., Blood 73 (1989) 1786-1793), which inhibits the ligand-receptor interaction, was used as reference control.

TABLE 2
Calculated IC50 values for the inhibition
of CSF-1 receptor phosphorylation.

| | IC50 CSF-1R |
|---|---|
| | Phosphorylation |
| CSF-1R Mab | [ng/ml] |
| Mab 2F11 | 219.4 |
| Mab 2E10 | 752.0 |
| Mab 2H7 | 703.4 |
| Mab 1G10 | 56.6 |
| SC-2-4A5 | 1006.6 |

### Example 4

Determination of the Binding of Anti-CSF-1R Antibodies to Human CSF-1R Fragment delD4 and to Human CSF-1R Extracellular Domain (CSF-1R-ECD) Preparation of Human CSF-1R Extracellular Domain (CSF-1R-ECD) (Comprising the Extracellular Subdomains D1-D5, hCSF-1R-ECD) of SEQ ID NO: 64:

pCMV-preS-Fc-hCSF-1R-ECD (7836 bp) encodes the complete ECD of human CSF-1R (SEQ ID NO: 64) C-terminally fused to a PreScission protease cleavage site, followed by aa100-330 of human IgG1 and a 6×His-Tag, under the control of CMV promoter. The natural signal peptide has been varied by insertion of amino acids G and S after the first M, in order to create a BamHI restriction site.

Preparation of human CSF-1R fragment delD4 (comprising the extracellular subdomains D1-D3 and D5, hCSF-1R-delD4) of SEQ ID NO: 65:

hCSF1R-delD4-V1-PreSc-hFc-His was cloned from pCMV-preS-Fc-hCSF-1R-ECD by means of the Stratagene QuikChange XL site-directed mutagenesis protocol, using delD4—for with sequence CACCTCCATGTTCTTCCGGTACCCCCCAGAGGTAAG (SEQ ID NO: 68) as the forward primer and delD4-rev with the reverse complement sequence as the reverse primer. A protocol variation published in BioTechniques 26 (1999) 680 was used to extend both primers in separate reactions in three cycles preceeding the regular Stratagene protocol:

Two separate 50 μl reaction mixtures were set up according to the manufacturer's manual, each containing 10 ng plasmid pCMV-preS-Fc-hCSF1R-ECD as the template and 10 pM of one of the primers delD4—for or delD4-rev, and 0.5 μl Pfu DNA polymerase as provided with the kit. Three PCR cycles 95° C. 30 sec/55° C. 60 sec/68° C. 8 min were run, then 25 μl each of both reaction mixtures were combined in a new tube and 0.5 μl fresh Pfu DNA polymerase were added. The regular PCR protocol with 18 temperature cycles as specified by Stratagene in the kit manual was carried out, followed by 2 hrs final digestion with the Dpn1 restriction enzyme provided with the kit. Clones bearing the deletion were detected by digestion with Cel II and Not I and verified by sequencing.

Protein was prepared by transient transfection in the Hek293 FreeStyle suspension cell system (Invitrogen) according to the manufacturer's specifications. After 1 week 500 ml supernatant was filtered and loaded onto a 1 ml HiTrap MabSelect Xtra (GE healthcare) protein A column (0.2 ml/min). The column was washed first with PBS, then with 50 mM Tris/150 mM NaCl/1 mM EDTA/pH 7.3. 75 μl PreScission Protease (GE #27-0843-01) diluted in 375 μl of the same buffer were loaded onto the column and the closed column was incubated over night at 4° C. with rolling. The column was mounted on top of a 1 ml GSTrap FF column (GE healthcare) and the desired protein was eluted (0.2 ml/min, 0.2 ml fractions). Pooled fractions were concentrated from 1.8 ml to 0.4 ml by centrifugal ultrafiltration via a 3k Nanosep and chromatographed over an 5200 HR SEC in PBS (0.5 ml/min). Human CSF-1R fragment delD4 was obtained in two fractions as a dimeric molecule (pool1, V=1.5 ml; c=0.30 mg/ml; apparent mass on SDS page 83 kDa, reduced 62 kDa) and as the monomer (pool 2, V=1.4 ml; c=0.25 mg/ml apparent mass on SDS page 62 kDa). The dimeric form was used for all experiments.

Determination of the Binding of Anti-CSF-1R Antibodies to Human CSF-1R Fragment delD4 and to Human CSF-1R Extracellular Domain (CSF-1R-ECD) (Binding Signals as Response Units (RU):

### Instrument: Biacore T100 (GE Healthcare)

Software: T100 Control, Version 2.0.1
   T100 Evaluation, Version 2.0.2

### Assayformat Chip: CMS

### Temperature: 25° C.

CSF-1R fragments were immobilized via amine coupling. To compare the binding of different anti-CSF-1R antibodies according to the invention one concentration of the test antibody was injected. Anti CSF-1R Mab3291 (R&D-Systems) and SC 2-4A5 (Santa Cruz Biotechnology, US—see also Sherr, C. J. et al., Blood 73 (1989) 1786-1793), was used as reference control, anti-CCR5 m<CCR5>Pz03.1C5 (deposited as DSM ACC 2683 on 18.08.2004 at DSMZ) as negative control, all under the same conditions as the anti-CSF-1R antibodies according to the invention.

### Amine Coupling of CSF-1R Fragments

Standard amine coupling according to the manufacturer's instructions: running buffer: PBS-T (Roche: 11 666 789+0.05% Tween20: 11 332 465), activation by mixture of EDC/NHS, injection of human CSF-1R fragment delD4 (comprising the extracellular subdomains D1-D3 and D5) (SEQ ID NO: 65) and human CSF-1R Extracellular Domain (CSF-1R-ECD) (comprising the extracellular subdomains D1-D5) (SEQ ID NO: 64) for 600 seconds at flow rate 10 μl/min; diluted in coupling buffer NaAc, pH 5.0, c=10 μg/mL; finally remaining activated carboxyl groups were blocked by injection of 1 M Ethanolamin.

Binding of <CSF-1R> Mab 2F11, Mab 2E10, Mab 3291 and Sc2-4A5 and Other Anti-CSF-1R Antibodies to Human CSF-1R Fragment delD4 and Human CSF-1R Extracellular Domain (CSF-1R-ECD) at 25° C.

Running buffer: PBS-T (Roche: 11 666 789+0.05% Tween20: 11 332 465) Analyte sample:

Binding was measured at a flow rate of 30 μL/min by one injection of the analyte with concentration c=10 nM. (for Mab 1G10, Mab 2H7 and humanized hMab 2F11-e7 in second experiment) Each injection was 700 seconds long, followed by a dissociation phase of 180 seconds. Final regeneration was performed after each cycle using 50 mM NaOH, contact time 60 seconds, flow rate 30 μL/min. Signals were measured by a report point 10 seconds after end of injection. Reference signals (signals from a blank reference flow cell (treated with EDC/NHS and ethanolamine, only) were subtracted to give the binding signals (as RU). If binding signals of nonbinding antibodies were slightly below 0 (Mab 2F11=−3; Mab 2E10=−2; Mab 1G10=−6, Mab 2H7=−9; and humanized hMab 2F11-e7=−7) the values were set as 0.

TABLE 3a
Binding of <CSF-1R> MAbs to human CSF-1R fragment delD4
and CSF-1R-ECD and ratio at 25° C., measured by SPR

| | Binding | Binding | Ratio of binding of |
|---|---|---|---|
| | to | to CSF- | anti-CSF1R antibodies |
| | delD4 | 1R-ECD | to CSF1R fragment |
| | [RU] | [RU] | delD4/to CSF-1R-ECD |
| Mab 3291 | 1015 | 627 | 1015/627 = 1.61 |
| sc2-4A5 | 374 | 249 | 374/249 = 1.50 |
| Mab 2F11 | 0 | 176 | 0/176 = 0 |
| hMab 2F11-e7 | 0 | 237 | 0/237 = 0 |
| Mab 2E10 | 0 | 120 | 0/120 = 0 |
| Mab 1G10 | 0 | 2708 | 0/2708 = 0 |
| Mab 2H7 | 0 | 147 | 0/147 = 0 |
| m<CCR5>Pz03.1C5 | 2 | 5 | — |

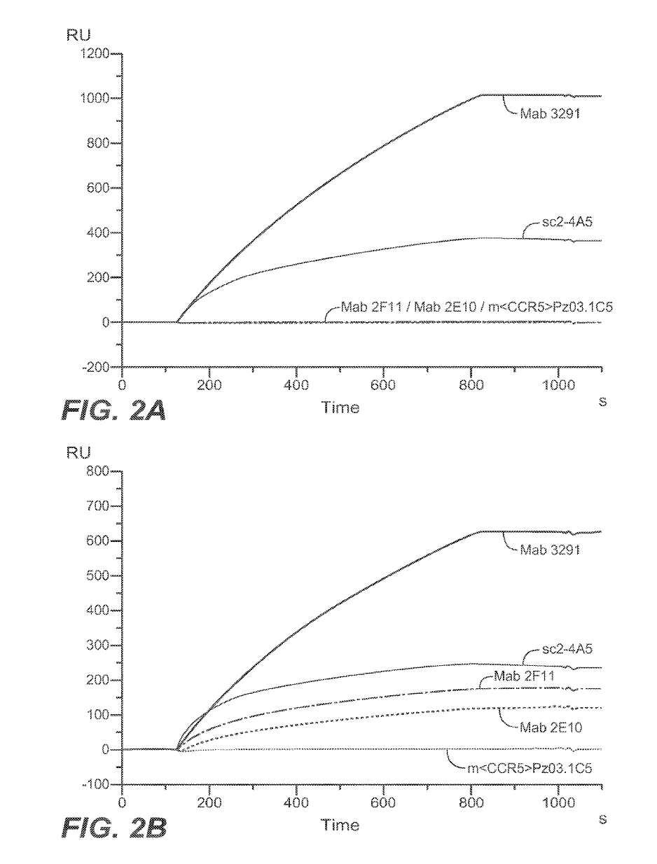

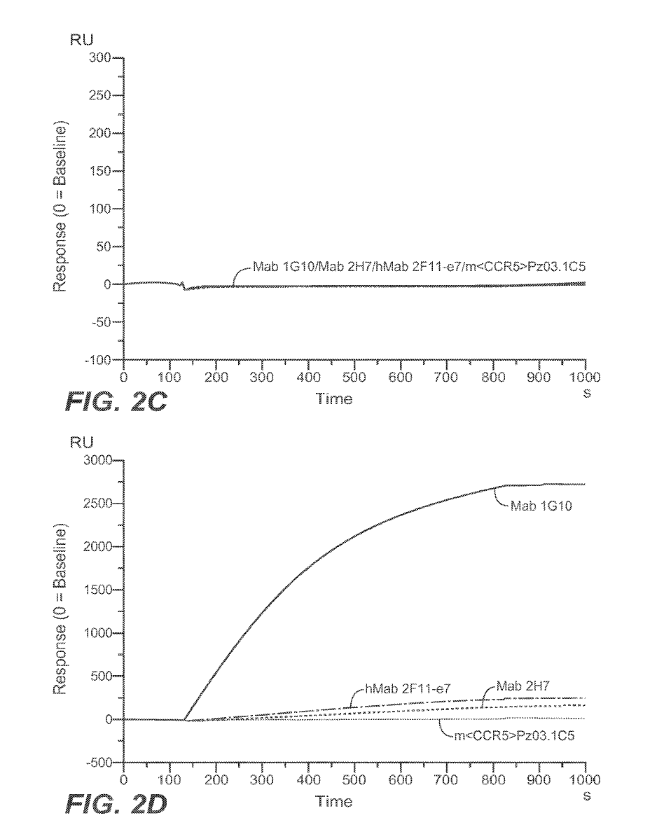

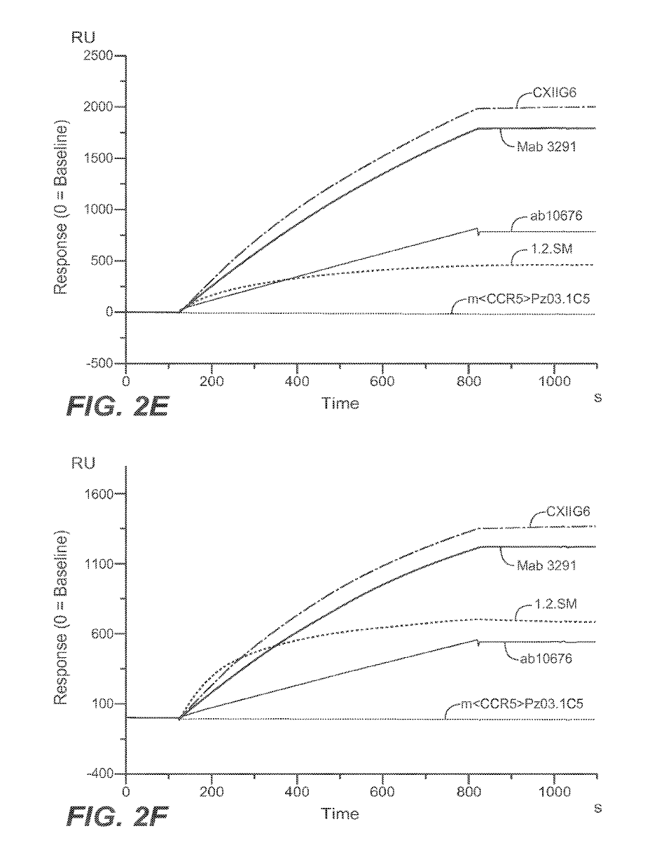

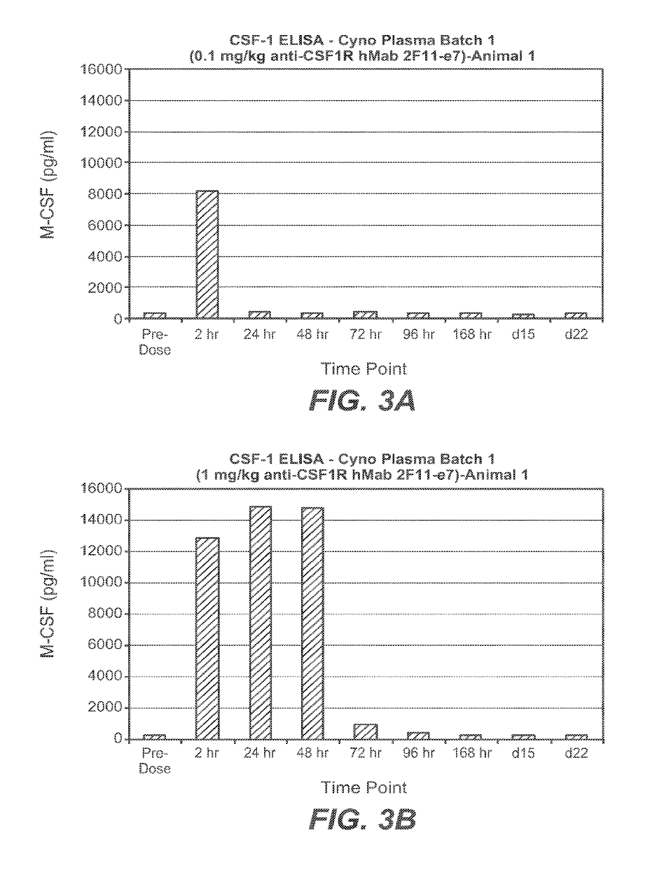

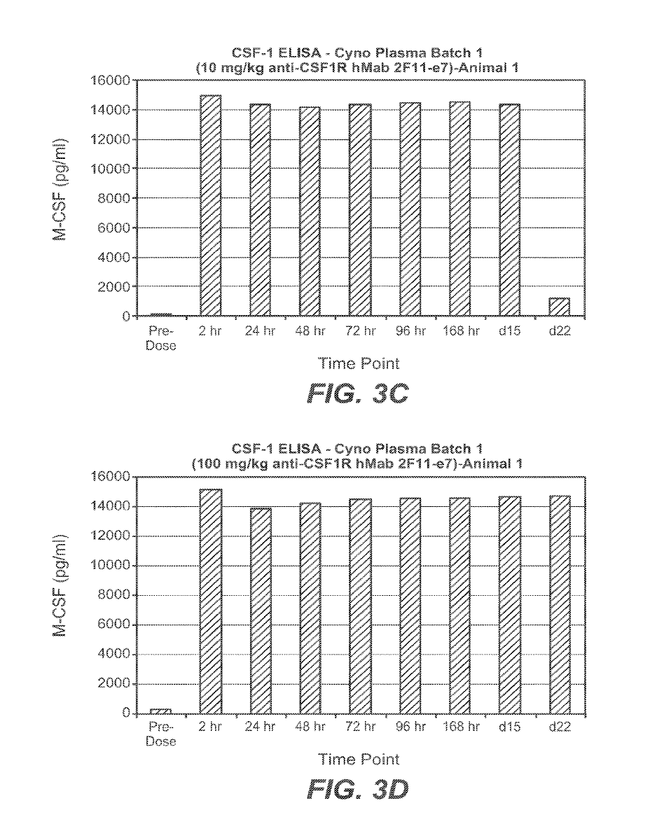

Mab 2F11 and Mab 2E10 showed binding to the human CSF-1R Extracellular Domain (CSF-1R-ECD) (see FIG. 2b); however no binding was detected to CSF-1R fragment delD4. (see FIG. 2a).

Sc2-4A5 and MAB3291 showed binding to CSF-1R-ECD and to del D4 (see FIGS. 2b and 2a).

Thus the ratio of binding of anti-CSF1R antibodies Mab 2F11 and Mab 2E10 to CSF1R fragment delD4/to CSF-1R-ECD was clearly below 1:50 (=0.02), while the binding ratio of MAB3291 and Sc2-4A5 were 1.61 and 1.50, respectively and were highly above 1:50 (=0.02). Negative control antibody m<CCR5>Pz03.1C5 did not show any binding (as expected).

Mab 1G10, Mab 2H7 and humanized hMab 2F11-e7 showed binding to the human CSF-1R Extracellular Domain (CSF-1R-ECD) (see FIG. 2d); however no binding was detected to CSF-1R fragment delD4. (see FIG. 2c). Thus the ratio of binding of anti-CSF1R antibodies Mab 1G10, Mab 2H7 and humanized hMab 2F11-e7 to CSF1R fragment delD4/to CSF-1R-ECD was clearly below 1:50 (=0.02). In a further experiment anti-CSF-1R antibodies 1.2.SM (ligand displacing CSF-1R antibody described in WO2009026303), CXIIG6 (ligand displacing CSF-1R antibody described in WO 2009/112245), the goat polyclonal anti-CSF-1R antibody ab10676 (abcam) were investigated. Anti-CSF-1R antibody Mab3291 (R&D-Systems) was used as reference control. Anti-CCR5 m<CCR5>Pz03.1C5 (deposited as DSM ACC 2683 on 18.08.2004 at DSMZ) was used as negative control.

TABLE 3b
Binding of <CSF-1R> MAbs to human CSF-1R fragment delD4 and
CSF-1R-ECD and ratio at 25° C., measured by SPR

| | Binding | Binding | Ratio of binding of |
|---|---|---|---|
| | to | to CSF- | anti-CSF1R antibodies |
| | delD4 | 1R-ECD | to CSF1R fragment |
| | [RU] | [RU] | delD4/to CSF-1R-ECD |
| MAB3291 | 1790 | 1222 | 1790/1222 = 1.47 |
| 1.2.SM | 469 | 704 | 469/704 = 0.67 |
| CXIIG6 | 1983 | 1356 | 1983/1356 = 1.46 |
| ab10676 | 787 | 547 | 787/547 = 1.44 |
| m<CCR5>Pz03.1C5 | 0 | 0 | — |

1.2.SM, CXIIG6, ab10676 and MAB3291 showed binding to CSF-1R-ECD and to del D4 (see FIGS. 2f and 2e).

The binding ratio of 1.2.SM, CXIIG6, ab10676 and MAB3291 was highly above 1:50 (=0.02). Negative control antibody m<CCR5>Pz03.1C5 did not show any binding (as expected).

### Example 5

Growth Inhibition of NIH3T3-CSF-1R Recombinant Cells in 3D Culture Under Treatment with Anti-CSF-1R Monoclonal Antibodies (CellTiterGlo-Assay)

NIH 3T3 cells, retrovirally infected with either an expression vector for full-length wildtype CSF-1R (SEQ ID NO: 62) or mutant CSF-1R L301S Y969F (SEQ ID NO: 63), were cultured in DMEM high glucose media (PAA, Pasching, Austria) supplemented with 2 mM L-glutamine, 2 mM sodium pyruvate and non-essential amino acids and 10% fetal bovine serum (Sigma, Taufkirchen, Germany) on poly-HEMA (poly(2-hydroxyethylmethacrylate)) (Polysciences, Warrington, Pa., USA)) coated dishes to prevent adherence to the plastic surface. Cells are seeded in medium replacing serum with 5 ng/ml sodium selenite, 10 mg/ml transferrin, 400 μg/ml BSA and 0.05 mM 2-mercaptoethanol. When treated with 100 ng/ml hu CSF-1 (active 149 aa fragment of human CSF-1 (aa 33-181 of SEQ ID NO: 86); Biomol, DE, Cat. No. 60530) wtCSF-1R (expressing cells form dense spheroids that grow three dimensionally, a property that is called anchorage independence. These spheroids resemble closely the three dimensional architecture and organization of solid tumors in situ. Mutant CSF-1R recombinant cells are able to form spheroids independent of the CSF-1 ligand. Spheroid cultures were incubated for 3 days in the presence of different concentrations of antibody in order to determine an IC50 (concentration with 50 percent inhibition of cell viability). The CellTiterGlo assay was used to detect cell viability by measuring the ATP-content of the cells.

TABLE 5a

| CSF-1R | wtCSF-1R | Mutant CSF-1R |
|---|---|---|
| Mab | IC₅₀ [μg/ml] | IC₅₀ [μg/ml] |
| Mab 2F11 | 1.1 | 8.0 |
| Mab 2E10 | 0.49 | 4.9 |
| Mab 2H7 | 0.31 | 5.3 |
| Mab 1G10 | 0.29 | 14.2 |
| SC 2-4A5 | 10.0 | 10.0 |

Reference control Mab R&D-Systems 3291 did not show inhibition of mutant CSF-1R recombinant cell proliferation.

In a further experiment the anti-CSF-1R antibody according to the invention hMab 2F11-e7 and the anti-CSF-1R antibodies 1.2.SM (ligand displacing CSF-1R antibody described in WO 2009/026303), CXIIG6 (ligand displacing CSF-1R antibody described in WO 2009/112245), the goat polyclonal anti-CSF-1R antibody ab10676 (abcam), and SC 2-4A5 (Santa Cruz Biotechnology, US— see also Sherr, C. J. et al., Blood 73 (1989) 1786-1793) were investigated. Spheroid cultures were incubated for 3 days in the presence of different concentrations of antibody in order to determine an IC30 (concentration with 30 percent inhibition of cell viability). Maximum concentration was 20 μg/ml The CellTiterGlo assay was used to detect cell viability by measuring the ATP-content of the cells.

TABLE 5b

| CSF-1R | wtCSF-1R | Mutant CSF-1R |
|---|---|---|
| Mab | IC₃₀ [μg/ml] | IC₃₀ [μg/ml] |
| hMab 2F11-e7 | 4.91 | 0.54 |
| 1.2.SM | 1.19 | >20 ug/ml (−19% |
| | | inhibition at 20 μg/ml = |
| | | 19% stimulation) |
| CXIIG6 | >20 ug/ml (21% | >20 ug/ml (−36% |
| | inhibition at 20 | inhibition at 20 μg/ml = |
| | μg/ml) | 36% stimulation) |
| ab10676 | 14.15 | >20 μg/ml (0% |
| | | inhibition at 20 μg/ml) |
| SC 2-4A5 | 16.62 | 2.56 |

### Example 6

Growth Inhibition of BeWo Tumor Cells in 3D Culture Under Treatment with Anti-CSF-1R Monoclonal Antibodies (CellTiterGlo-Assay)

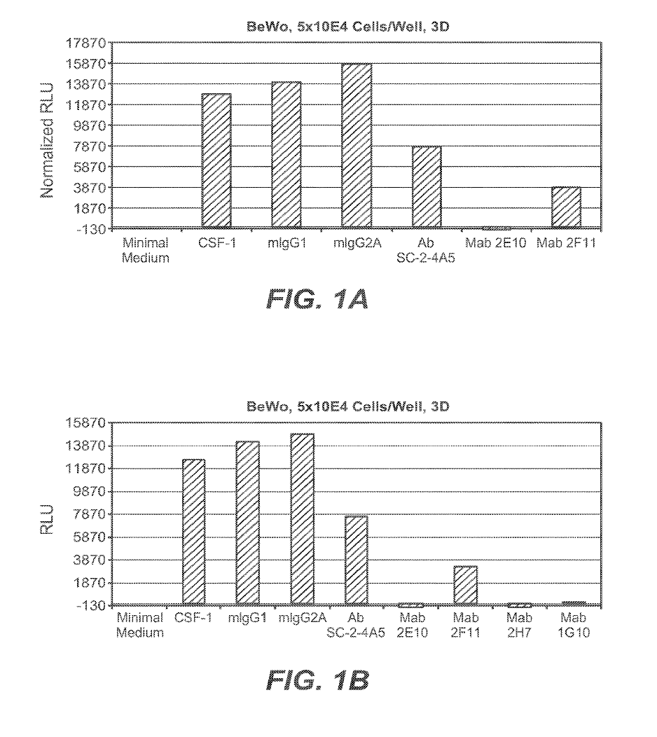

BeWo choriocarcinoma cells (ATCC CCL-98) were cultured in F12K media (Sigma, Steinheim, Germany) supplemented with 10% FBS (Sigma) and 2 mM L-glutamine. 5×10⁴ cells/well were seeded in 96-well poly-HEMA (poly(2-hydroxyethylmethacrylate)) coated plates containing F12K medium supplemented with 0.5 FBS and 5% BSA. Concomitantly, 200 ng/ml huCSF-1 (active 149 aa fragment of human CSF-1 (aa 33-181 of SEQ ID NO: 86)) and 10 μg/ml of different anti-CSF-1R monoclonal antibodies were added and incubated for 6 days. The CellTiterGlo assay was used to detect cell viability by measuring the ATP-content of the cells in relative light units (RLU). When BeWo spheroid cultures were treated with different anti-CSF-1R antibodies (10 μg/ml) inhibition of CSF-1 induced growth was observed. To calculate antibody-mediated inhibition the mean RLU value of unstimulated BeWo cells was subtracted from all samples. Mean RLU value of CSF-1 stimulated cells was set arbitrarily to 100%. Mean RLU values of cells stimulated with CSF-1 and treated with anti-CSF-1R antibodies were calculated in % of CSF-1 stimulated RLUs. The Table 6 shows the calculated data of growth inhibition of BeWo tumor cells in 3D culture under treatment with anti-CSF-1R monoclonal antibodies; FIGS. 1a and b depicts normalized mean RLU values.

TABLE 6

| | % inhibition 10 μg/ml |
|---|---|
| CSF-1R Mab | antibody concentration |
| CSF-1 only | 0 |
| Mab 2F11 | 70 |
| Mab 2E10 | 102 |
| Mab 2H7 | 103 |
| Mab 1G10 | 99 |
| SC 2-4A5 | 39 |

### Example 7

Inhibition of Human Macrophage Differentiation Under Treatment with Anti-CSF-1R Monoclonal Antibodies (CellTiterGlo-Assay)

Human monocytes were isolated from peripheral blood using the RosetteSep™ Human Monocyte Enrichment Cocktail (StemCell Tech.—Cat. No. 15028). Enriched monocyte populations were seeded into 96 well microtiterplates (2.5×10⁴ cells/well) in 100 μl RPMI 1640 (Gibco—Cat. No. 31870) supplemented with 10% FCS (GIBCO—Cat. No. 011-090014M), 4 mM L-glutamine (GIBCO—Cat. No. 25030) and 1× PenStrep (Roche Cat. No. 1 074 440) at 37° C. and 5% CO₂ in a humidified atmosphere. When 150 ng/ml huCSF-1 was added to the medium, a clear differentiation into adherent macrophages could be observed. This differentiation could be inhibited by addition of anti-CSF-1R antibodies. Furthermore, the monocyte survival is affected and could be analyzed by CellTiterGlo (CTG) analysis. From the concentration dependent inhibition of the survival of monocytes by antibody treatment, an IC₅₀ was calculated (see Table 7).

TABLE 7

| CSF-1R Mab | IC₅₀ [μg/ml] |
|---|---|
| Mab 2F11 | 0.08 |
| Mab 2E10 | 0.06 |
| Mab 2H7 | 0.03 |
| Mab 1G10 | 0.06 |
| SC 2-4A5 | 0.36 |

In a separate test series humanized versions of Mab 2 F11, e.g. hMab 2F11-c11, hMab 2F11-d8, hMab 2F11-e7, hMab 2F11-f12, showed IC50 values of 0.07 μg/ml (hMab 2F11-c11), 0.07 μg/ml (hMab 2F11-d8), 0.04 μg/ml (hMab 2F11-e7) and 0.09 μg/ml (hMab 2F11-f12).

### Example 8

Inhibition of Cynomolgous Macrophage Differentiation Under Treatment with Anti-CSF-1R Monoclonal Antibodies (CellTiterGlo-Assay)

Cynomolgous monocytes were isolated from peripheral blood using the CD14 MicroBeads non-human primate kit (Miltenyi Biotec—Cat. No. 130-091-097) according to the manufacturers description. Enriched monocyte populations were seeded into 96 well microtiterplates (1-3×10⁴ cells/well) in 100 μl RPMI 1640 (Gibco—Cat. No. 31870) supplemented with 10% FCS (GIBCO—Cat. No. 011-090014M), 4 mM L-glutamine (GIBCO—Cat. No. 25030) and 1× PenStrep (Roche Cat. No. 1 074 440) at 37° C. and 5% CO₂ in a humidified atmosphere. When 150 ng/ml huCSF-1 was added to the medium, a clear differentiation into adherent macrophages could be observed. This differentiation could be inhibited by addition of anti-CSF-1R antibodies. Furthermore, the monocyte survival is affected and could be analyzed by CellTiterGlo (CTG) analysis. The viability was analyzed at a concentration of 5 μg/ml antibody treatment (see Table 8).

TABLE 8

| CSF-1R | | % inhibition (of survival) = |
|---|---|---|
| Mab | % survival | (100%-% survival) |
| Mab 2F11 | 4 * | 96 |
| Mab 2E10 | 17 ** | 83 |
| Mab 2H7 | 8 | 92 |
| Mab 1G10 | 2 | 98 |
| SC 2-4A5 | 31 | 69 |

* mean of four experiments (3 expts. using the murine, 1 expt. using the chimeric mAb)
** mean of two experiments using the murine mAb only

### Example 9

Inhibition of Human M1 and M2 Macrophage Differentiation Under Treatment with Anti-CSF-1R Monoclonal Antibodies (CellTiterGlo-Assay)

Human monocytes were isolated from peripheral blood using the RosetteSep™ Human Monocyte Enrichment Cocktail (StemCell Tech.—Cat. No. 15028). Enriched monocyte populations were seeded into 96 well microtiterplates (2.5×10⁴ cells/well) in 100 μl RPMI 1640 (Gibco—Cat. No. 31870) supplemented with 10% FCS (GIBCO—Cat. No. 011-090014M), 4 mM L-glutamine (GIBCO—Cat. No. 25030) and 1× PenStrep (Roche Cat. No. 1 074 440) at 37° C. and 5% CO₂ in a humidified atmosphere. When 100 ng/ml huCSF-1 was added for 6 days to the medium, a clear differentiation into adherent, M2 macrophages with elongated morphology could be observed. When 100 ng/ml huGM-CSF was added to the medium for 6 days, a clear differentiation into adherent, M1 macrophages with round morphology could be observed. This differentiation was associated with the expression of certain markers such as CD163 for M2 macrophages and CD80 or high MHC class II for M1 macrophages as assessed by flow cytometry. Cells were washed with PBS and, if adherent, detached using a 5 mM EDTA solution in PBS (20 min at 37° C.). Cells were then well resuspended, washed with staining buffer (5% FCS in PBS) and centrifuged at 300×g for 5 min Pellets were resuspended in 1 ml staining buffer and cells counted in a Neubauer chamber. Approximately 1×10e5 cells were transferred in each FACS tube, centrifuged at 300×g for 5 min and resuspended in staining buffer. Fcγ receptors were blocked by incubation with 1 μg human IgG/2.5×10e4 cells (JIR Cat. No. 009-000-003) in staining buffer for 20 min on ice. Cells were then mixed with 1.5 μl antibody/2.5×10e4 cells for CD80 and CD163 detection whereas 5 μl antibody/2.5×10e4 cells for MHC class II detection was used: PE labeled mouse anti human CD163 (BD Bioscience Cat. No. 556018), PE labeled mouse anti human CD80 (BD Bioscience Cat. No. 557227) and Alexa 647 labeled mouse anti human MHC class II (Dako—Cat. No. M0775). The Alexa 647 label was conjugated to the antibody by using the Zenon Alexa 647 mouse IgG labeling kit (Invitrogen Cat. No. Z25008) After a 1-hour incubation on ice cells were washed twice with staining buffer, resuspended and measured at a FACS Canto II.

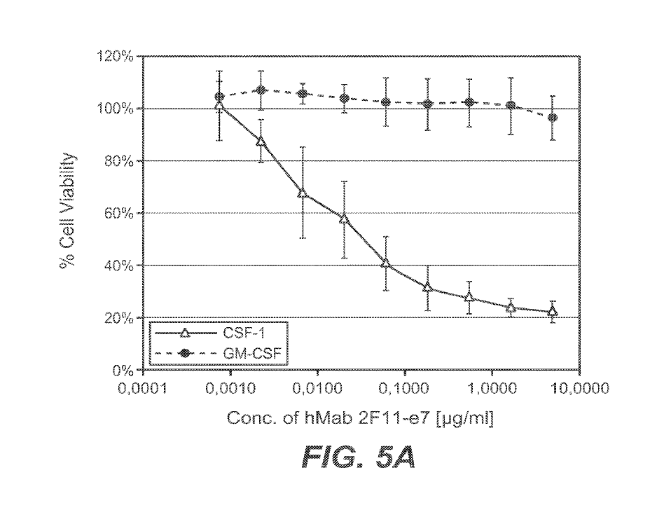

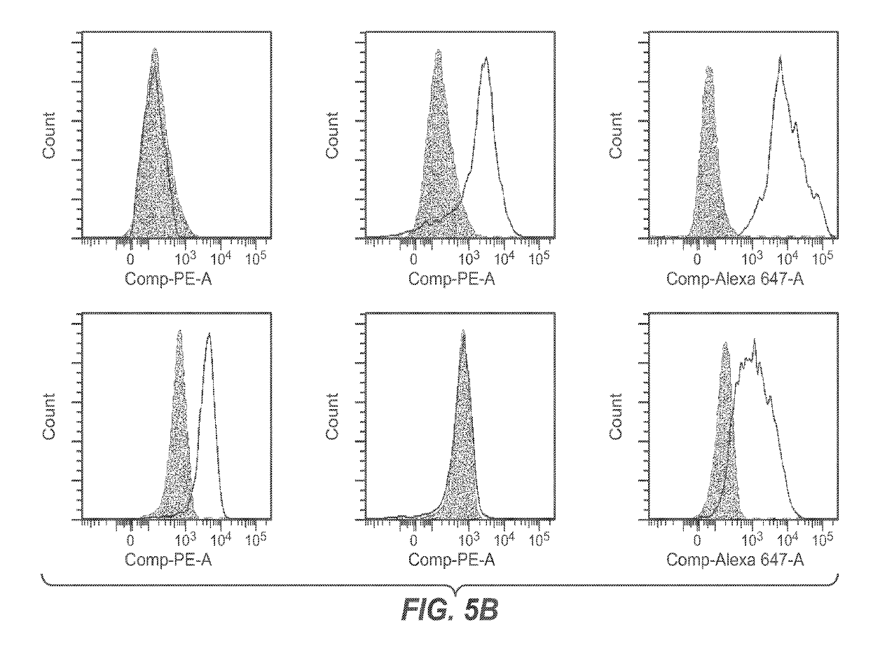

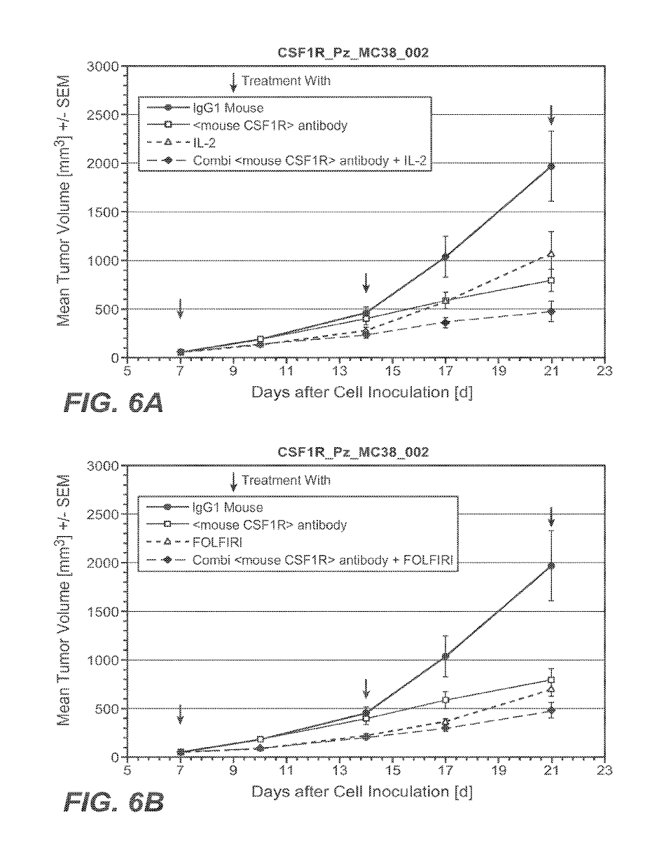

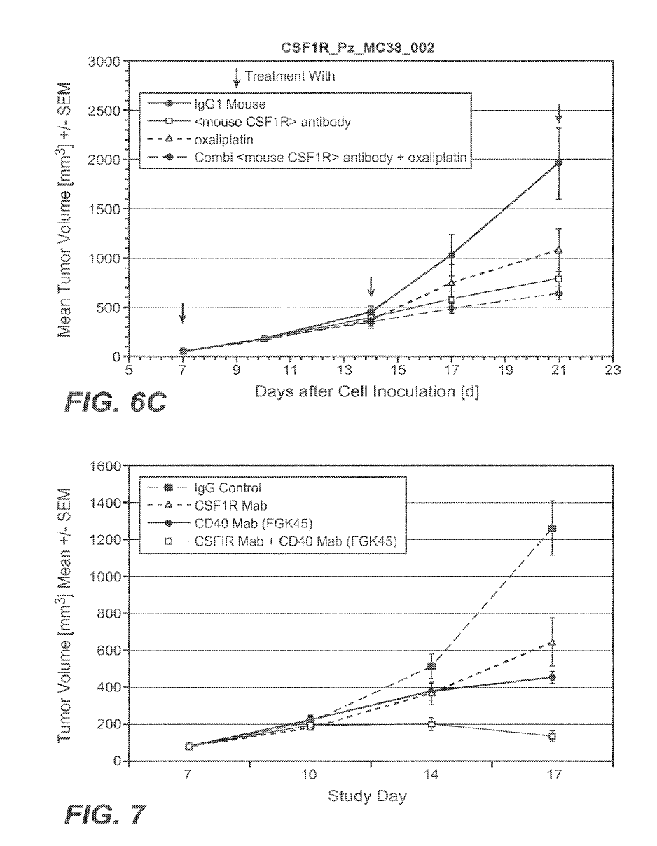

Exclusively M2 macrophage differentiation which is characterized by the expression of CD163, absence of CD80 and low MHC class II expression could be inhibited by addition of humanized anti-CSF-1R antibody hMab 2F11-e7. Furthermore, the M2 but not M1 macrophage survival is affected and could be analyzed by CellTiterGlo (CTG) analysis. Concentration dependent inhibition of the survival of macrophages by antibody treatment for 7 days is depicted in FIG. 5a. Expression of M1 and M2 macrophage markers assessed by flow cytometry is shown in FIG. 5b.

### Example 10

### Determination of the Binding Affinity of Anti-CSF-1R Antibodies to Human CSF-1R

### Instrument: BIACORE® A100

### Chip: CMS (Biacore BR-1006-68)

Coupling: amine coupling

### Buffer: PBS (Biacore BR-1006-72), pH 7.4, 35° C.

For affinity measurements 36 μg/ml anti mouse Fcγ antibodies (from goat, Jackson Immuno Research JIR115-005-071) have been coupled to the chip surface for capturing the antibodies against CSF-1R. Human CSF-1R Extracellular Domain (CSF-1R-ECD) (comprising the extracellular subdomains D1-D5) (SEQ ID NO: 64) (R&D-Systems 329-MR or subcloned pCMV-presS-HisAvitag-hCSF-1R-ECD) was added in various concentrations in solution. Association was measured by an CSF-1R-injection of 1.5 minutes at 35° C.; dissociation was measured by washing the chip surface with buffer for 10 minutes at 35° C. For calculation of kinetic parameters the Langmuir 1:1 model was used.

TABLE 9
Affinity data measured by SPR

| CSF-1R Mab | K_{D} (nM) | kₐ (1/Ms) | k_{d} (1/s) | t_{1/2}(min) |
|---|---|---|---|---|
| Mab 2F11 | 0.29 | 1.77E⁺⁰⁵ | 5.18E⁻⁰⁵ | 223 |
| Mab 2E10 | 0.2 | 1.52E⁺⁰⁵ | 2.97E⁻⁰⁵ | 389 |
| Mab 2H7 | 0.21 | 1.47E⁺⁰⁵ | 3.12E⁻⁰⁵ | 370 |
| Mab 1G10 | 0.36 | 1.75E⁺⁰⁵ | 6.28E⁻⁰⁵ | 184 |

In a separate biacore binding assay using the CSF-1R ECD (data not shown) some competition of the antibodies Mab 2F11 and Mab 2E10 with the antibody Ab SC-2-4A5 was shown. However Mab 2F11/Mab 2E10 do not bind to the human CSF-1R fragment delD4, whereas Ab SC-2-4A5 binds to this delD4 fragment (see Example 4 and FIG. 2a). Thus the binding region of Mab 2F11/Mab 2E10 is clearly distinct from the binding region of Ab SC-2-4A5, but probably located in a vicinity area. In such competition assay both antibodies Mab 2F11 and Mab 2E10 did not compete with Mab3291 from R&D-Systems (data not shown).

### Example 11

### Determination of the Binding of Anti-CSF-1R Antibodies to Human CSF-1R Fragment D1-D3

### Instrument: Biacore T100 (GE Healthcare)

Software: T100 Control, Version 1.1.11
   B3000 Evaluation, Version 4.01
   Scrubber, Version 2.0a

### Assayformat Chip:CMS-Chip

Antibodies against CSF-1R were captured via amine coupled capture molecules.

Using the single cycle kinetics five increasing concentrations of human CSF-1R fragment D1-D3 (SEQ ID NO: 66) were injected. Human CSF-1R fragment D1-D3 was subcloned into pCMV-presS-HisAvitag expression vector.

Anti CSF-1R SC 2-4A5 (Santa Cruz Biotechnology, US; Sherr, C. J. et al., Blood 73 (1989) 1786-1793) which inhibits the ligand-receptor interaction, and Mab 3291 (R&D-Systems) were used as reference controls.

Capture molecules: Anti mouse Fcγ antibodies (from goat, Jackson Immuno Research JIR115-005-071) for antibodies according to the invention and the R&D-Systems control Mab 3291 and Anti rat Fcγ antibodies (from goat, Jackson Immuno Research JIR112-005-071) for the reference control anti CSF-1R SC 2-4A5.

### Amine Coupling of Capture Molecules

Standard amine coupling according to the manufacturer's instructions: running buffer: HBS-N buffer, activation by mixture of EDC/NHS, aim for ligand density of 2000 RU; the capture-Abs were diluted in coupling buffer NaAc, pH 4.5, c=10 μg/mL; finally remaining activated carboxyl groups were blocked by injection of 1 M Ethanolamin.

### Kinetic Characterization of Human CSF-1R Fragments D1-D3 Binding to MAbs <CSF-1R> at 37° C.

Running buffer: PBS (Biacore BR-1006-72)
Capturing of Mabs <CSF-1R> on flow cells 2 to 4: Flow 20 μL/min, contact time 90 seconds, c(Abs<CSF-1R>)=50 nM, diluted with running buffer+1 mg/mL BSA;
Analyte sample:

Single Cycle Kinetics was measured at a flow rate of 30 μL/min by five consecutive injections of the analyte with concentrations, c=7.8, 31.25, 125 500 and 2000 nM, without regeneration. Each injection was 30 seconds long and followed by a dissociation phase of 120 Seconds for the first four injections, and finally 1200 seconds for the highest concentration (=last injection).

Final regeneration was performed after each cycle using 10 mM Glycin pH 1.5 (Biacore BR-1003-54), contact time 60 seconds, flow rate 30 μL/min

Kinetic parameters were calculated by using the usual double referencing (control reference: binding of analyte to capture molecule; Flow Cell: subdomain CSF-1R concentration “0” as Blank) and calculation with model ‘titration kinetics 1:1 binding with draft’.

TABLE 10
Affinity data for binding of human CSF-1R
fragment D1-D3 measured by SPR

| CSF-1R | Sub | | | | |
|---|---|---|---|---|---|
| Mab | domain | K_{D} (nM) | kₐ (1/Ms) | k_{d} (1/s) | tₘᵢₙ (min) |
| Mab 2F11 | D1-D3 | no binding | | | |
| Mab 2E10 | D1-D3 | no binding | | | |
| Mab 2H7 | D1-D3 | not | | | |
| | | determined | | | |
| Mab 1G10 | D1-D3 | no binding | | | |
| SC-2-4A5 | D1-D3 | no binding | | | |
| R&D-Systems | D1-D3 | 5.4 | 2.2E⁺⁵ | 1.2E⁻³ | 9.6 |
| 3291 | | | | | |

The antibodies Mab 2F11, Mab 2E10 and Mab 1G10 showed no binding to human CSF-1R fragment D1-D3.

Also reference control-Ab SC-2-4A5 did not bind to human CSF-1R fragment D1-D3.

The reference control Mab R&D-Systems 3291 showed binding to the human CSF-1R fragment D1-D3.

### Example 12

### CSF-1 Level Increase During CSF-1R Inhibition in Cynomolgus Monkey

Serum CSF-1 levels provide a pharmacodynamic marker of CSF-1R neutralizing activity of anti-human CSF-1R dimerization inhibitor hMab 2F11-e7. One male and one female cynomolgous monkey per dosage group (1 and 10 mg/kg) were intravenously administered anti-CSF1R antibody hMab 2F11-e7. Blood samples for analysis of CSF-1 levels were collected 1 week before treatment (pre-dose), 2, 24, 48, 72, 96, 168 hours post-dose and weekly for two additional weeks. CSF-1 levels were determined using a commercially available ELISA kit (Quantikine® human M-CSF) according to the manufacturer's instructions (R&D Systems, UK). Monkey CSF-1 level were determined by comparison with CSF-1 standard curve samples provided in the kit.

Administration of hMab 2F11-e7 induced a dramatic increase in CSF-1 by ˜1000-fold, which depending on the dose administered lasted for 48 hr (1 mg/kg) or 15 days (10 mg/kg). Hence, a dimerization inhibitor for CSF-1R offers the advantage to not directly compete with the dramatically upregulated ligand for binding to the receptor in contrast to a ligand displacing antibody.

### Example 13

### In Vivo Efficacy—Tumor Growth Inhibition of Anti-CSF-1R Antibodies in Breast Cancer BT20 Xenograft Tumor Cells in SCID Beige Mice

The human breast cancer cell line BT-20 expresses human CSF-1R but lacks CSF-1 expression (Sapi, E. et al Cancer Res 59 (1999) 5578-5585). Since the mouse derived CSF-1 fails to activate human CSF-1R on the tumor cells recombinant human CSF-1 (active 149 aa fragment of human CSF-1 (aa 33-181 of SEQ ID NO: 86) (Biomol, Hamburg, Germany) was supplemented via osmotic minipumps (ALZET, Cupertino, Calif.) providing a continuous CSF-1 infusion rate of 2 μg/day (Martin, T. A., Carcinogenesis 24 (2003) 1317-1323).

To directly compare the efficacy of an antibody interfering with dimerization of CSF-1R with a ligand displacing CSF-1R antibody we tested the chimeric anti-CSF-1R Mab 2F11 (antibody interfering with dimerization of CSF-1R) and 1.2.SM (ligand displacing CSF-1R antibody described in WO 2009/026303) in the BT-20 xenograft model.

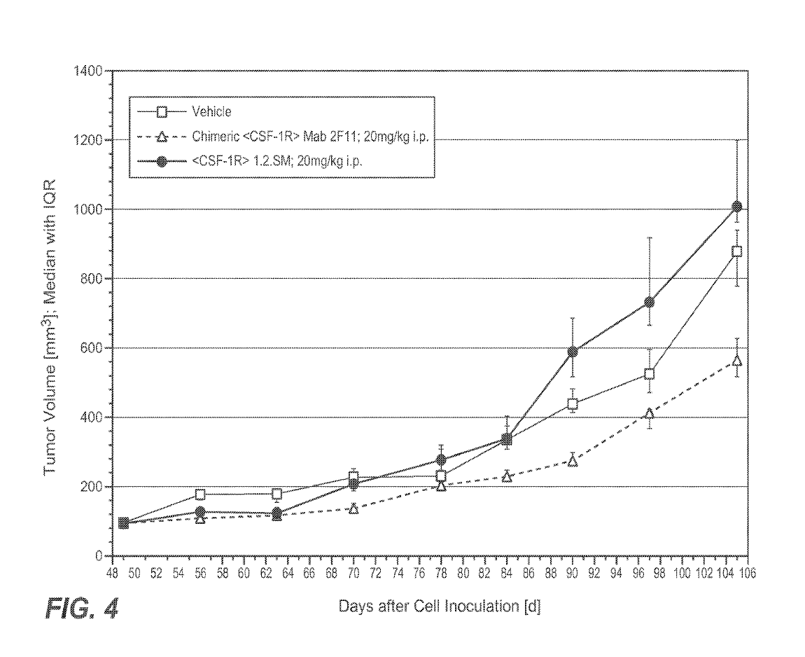

SCID beige mice (Charles River, Sulzfeld, Germany) were subcutaneously coinjected with 1×107 cells BT-20 cells (ATCC HTB-19) and 100 μl of Matrigel. Treatment of animals started at day of randomization at a mean tumor volume of 100 mm3. Mice are treated once weekly i.p. with the respective antibodies (see FIG. 4) in 20 mM Histidine, 140 mM NaCl pH 6.0 buffer. The tumor dimensions are measured by caliper beginning on the staging day and subsequently 2 times per week during the whole treatment period. Tumor volume is calculated according to NCI protocol (Tumor weight=1/2ab2, where “a” and “b” are the long and the short diameters of the tumor, respectively).

Tumor growth analysis is shown in FIG. 4. Inhibition of human CSF-1R on tumor cells with the chimeric anti-CSF-1R Mab 2F11 was statistically more efficacious in mediating tumor growth inhibition than anti-CSF-1R antibody 1.2.SM (CSF-1R antibody described in WO 2009/026303).

In a separate experiment 3 mg/kg i.v. docetaxel (Taxotere® Sanofi Aventis, UK) treatment was combined with anti-mouse CSF-1R antibody (30 mg/kg i.p/weekly). Docetaxel was administered 3 times weekly as 1 cycle followed by 3 weeks drug holiday. After 2 cycles of docetaxel treatment antibody monotherapy inhibited primary tumor growth (TGI: 83%, npTCR: 0.5, CI: 0.1-1.8) comparable to the 3 mg/kg docetaxol group (TGI: 75%, npTCR: 0.55, CI: 0.2-1.5). Combination of docetaxol and anti-CSF-1R antibody resulted in superior efficacy than the monotherapies (TGI: 94%, npTCR:0.3, CI: 0.1-0.8). At a later time point differences in TGI between combination and monotherapy groups were less pronounced due to the strong inhibition of each of the monotherapy. Nevertheless the analysis of median survival time revealed superiority of the combination (antibody 159d, docetaxel 154d, combination 180d).

### Example 14

Combination Treatment of an Anti-CSF-1R Antibodies Binding to the Domains D4 to D5 of the Extracellular Domain Human CSF-1R with Paclitaxel.

### 2.1 Primary Objectives

Part I (Arm A: humanized version of anti-CSF-1R Mab 2F11 (hMab 2F11-e7) single agent [SA] dose escalation; Arm B: humanized CSF-1R antibody Mab 2F11 (hMab 2F11-e7) dose escalation in combination [CD] with fixed dose of paclitaxel):
To evaluate the safety, tolerability and PK of humanized version of Mab 2F11 when administered alone and in combination with paclitaxel
To determine the maximum tolerated dose (MTD) and/or Optimal Biological Dose (OBD) of humanized Mab 2F11 when administered alone (MTD1/OBD1) and in combination with paclitaxel (MTD2/OBD2) by observing the dose-limiting toxicities (DLTs).

Part II (Expansion Cohorts/humanized Mab 2F11 single agent only): To extend safety assessment and investigate humanized Mab 2F11 clinical activity in patients with a tumor entity of particular interest based on observations in Part I of the study, all of whom are not amenable to standard treatment.

### 2.2 Secondary Objectives

### Part I (Dose Escalation/Arm A+B)

To explore the PK and PD effects of humanized Mab 2F11 alone and in combination with paclitaxel in the tumor and surrogate tissue
To assess the PD and biomarker effects of humanized Mab 2F11 alone and in combination with paclitaxel as measured by changes in 18F Fluoro-Deoxy-Glucose Positron Emission Tomography (FDG-PET) and Dynamic Contrast-Enhanced Ultrasound (DCE-US) (where available)
To identify the recommended Phase 2 dose (RP2D) and schedules for humanized CSF-1R antibody Mab 2F11 alone and in combination with paclitaxel
To explore preliminary clinical activity of humanized Mab 2F11 alone and in combination with paclitaxel, using Objective Response Rate (ORR), Clinical Benefit Rate (CBR), Progression-free survival (PFS), Duration of response.

### Part II (Expansion Cohorts/Arm A Only)

To further characterize the PK and PD effects of humanized Mab 2F11 in the tumor and surrogate tissue

### 2.3 Exploratory Objectives

Collected patient Specimens will be analysed to:
Retrospectively identify TAM dependent tumors
Explore possible response prediction markers in surrogate tissue like skin and blood
Study the association of biomarkers with efficacy and/or adverse events (AEs) associated with medicinal products; and/or
Develop biomarker or diagnostic assays;

### 3. Study Design

### 3.1 Overview of Study Design

This is an open-label, multicenter, Phase Ia/b dose escalation study designed to assess the safety, tolerability, PK and PD of every two weeks (Q2W) i.v. dosing of humanized Mab 2F11. humanized Mab 2F11 will be administered alone for patients with solid tumors (which are not amenable to standard treatment and in combination with paclitaxel in locally advanced and/or metastatic carcinoma which are not amenable to standard treatment.

### Part I—Dose Escalation

All patients enrolled in the dose escalation cohorts will be assessed for DLTs during a DLT assessment period of 28 days following the first administration of humanized Mab 2F11 in Cycle 1. Patients who discontinue for any reason other than DLT during the DLT assessment period will be replaced.

Humanized Mab 2F11 Monotherapy Administration Mode Humanized Mab 2F11 will be administered Q2W as i.v. infusion over 1.5 h, unless the patient experiences an infusion-related reaction (IRR) which would require slowing or temporary halting of the infusion. Treatment will be administered until disease progression, unacceptable toxicity, death or patient refusal, whichever occurs first.

Humanized Mab 2F11 and Paclitaxel Combination Administration Mode (Part I, Arm B Only)

Humanized Mab 2F11 will be administered every Q2W as i.v. infusion over 1.5 h, unless the patient experiences an IRR which would require slowing or temporary halting of the infusion. Treatment will be administered until disease progression, unacceptable toxicity, death or patient refusal, whichever occurs first. Paclitaxel, at a dose of 80 mg/m2 will be administered QW for up to 12 weeks in combination with humanized Mab 2F11. The paclitaxel infusion will be started as soon as the humanized Mab 2F11 infusion has ended and will be administered according to local prescribing information. If a patient experiences toxicity directly attributable to paclitaxel, he/she may stop treatment with paclitaxel but continue to receive humanized Mab 2F11.

### Part I of the Trial Definition of MTD1/OBD1 and MTD2/OBD2

The first 28 days following the first administration of humanized Mab 2F11 in Cycle 1 will be considered the treatment interval for determination of DLT to define MTD1 and MTD2.

The MTD is defined as the highest dose level(s) at which no more than 1 out of 6 patients experiences a DLT.

Safety data and any available PK/PD data will be collected on an ongoing basis and reviewed prior to each dose escalation decision for the next cohort.

### 3.1.1 Rationale for Study Design

In-house screening of tumor biopsy samples from different patients with different malignancies has shown significant heterogeneity in the density of infiltrating macrophages and co-incident CSF-1R expression (Please see the non-clinical pharmacology section of the IB). Target-mediated drug disposition (TMDD), i.e. distribution and elimination via binding to the pharmacological target, was also clearly evident in monkey and both tumor-bearing and non-tumor bearing mice. Since the pharmacokinetics of humanized Mab 2F11 is affected by its binding to the target, the quantification of the nonlinear PK can be used as a biomarker to approximate target saturation. In order to characterise to what extent baseline patient demographic factors (including tumor mass and TAM density) may influence the non-linear pharmacokinetics of humanized Mab 2F11, blood levels will be measured within the first few days following a single low (100 mg) ‘run-in’ dose (cycle 0) in all patients from cohort 2 onwards (i.e. 1 week prior to their cycle 1 dose which will be at least 200 mg or higher). At this low dose, nonlinear PK is expected and this will allow quantification of the TMDD in cancer patients. The value of the run-in dose is that it will provide an understanding whether, in the extension phase of the trial (or future studies), different doses may be more effective in the different extension arms (i.e. different malignancies) based on patient demographic and baseline factors (including tumor type, size and inflammatory status). Since CSF-1R blockade has been demonstrated to selectively inhibit TAMs, thus offering the potential to prevent or event reverse TAM-mediated chemo-resistance [10], a concurrent assessment of humanized Mab 2F11 given in combination with paclitaxel will be initiated. Paclitaxel was chosen as a commonly prescribed chemotherapy for these patient groups and is not expected to produce significant overlapping toxicity, since the most commonly reported toxicities with paclitaxel (myelosuppression, neurotoxicity and arthralgia or myalgia) have not been reported in toxicity studies for A fixed dose of paclitaxel, given QW for up to 12 weeks, will be investigated in combination with ascending doses of humanized Mab 2F11 for patients with advanced breast or ovarian cancer. Recent data have shown that in patients with PVNS and TGCT, over-expression of CSF-1 is detected and is in part mediated by a translocation involving the CSF-1R gene in 30-60% of cases. Further, presence of CSF-1R positive macrophages in several other human cancers (such as ovarian and breast carcinoma) has been shown to correlate not only with increased vascular density but also worse clinical outcome. In breast cancer the presence of a CSF-1 response gene signature predicts risk of recurrence and metastasis. On the basis of these findings and our preclinical models, it seems reasonable to test the hypothesis that blockade of tumor associated macrophages and their pro-tumor bioactivity with humanized Mab 2F11 alone or in combination with paclitaxel has the potential to show clinical activity in patients with certain types of solid tumors.

This study contains a number of blood draws for assessment of PK and PD parameters as well as mandatory fresh and archival tumor tissue collection These are important in enabling a full understanding of the PK properties, mechanism of action and potential for predictive response biomarkers.

### 3.1.4 Rationale for Biomarker Evaluation

Biomarkers have the potential to shape diagnostic strategies and influence therapeutic management. In the future, biomarkers may promote a personalized medicine approach, grouping patients by the molecular signatures of their tumors and of markers in the blood rather than by cancer type. We are concentrating our efforts in identifying predictive biomarkers, which provide information about the likely efficacy and safety of the therapy. To evaluate the PD and mechanistic effect/s of a drug on the tumor a tumor biopsy is often required.

### 3.1.4.1 Rationale for Fresh Pre- and On-Treatment Tumor Biopsy

TAM infiltration and differentiation is dependent on the respective tumor micro-milieu in primary and metastatic lesions. Furthermore the respective immune status and pre-treatment of the patient might influence the patient's tumor microenvironment. Therefore all patients will undergo a mandatory pre-treatment biopsy to define the TAM infiltration and CSF-1R expression levels at baseline but will not be used to determine patient eligibility for the trial. In addition, mandatory on-treatment biopsies will allow the assessment of the PD activity of humanized Mab 2F11 by comparing pre- and post-dose levels. Fine Needle Aspiration (FNA) will not be suitable to substitute for tumor biopsies, as macrophage sub-population distribution needs to be assessed in the tissue.

Archival tumor tissue cannot substitute for the fresh biopsies as macrophage infiltration and differentiation is micro-milieu dependent. The tumor micro-milieu may be variable in the primary tumor due to pre-treatment of the patient and as well be altered in metastatic lesions. However, if archival tumor tissue is available, samples will be used for exploratory retrospective correlation of data with fresh biopsies

### 3.1.4.2 Rationale for Wounded Skin Biopsies

The different phases of wound healing require many processes (e.g. neutrophil recruitment, macrophage infiltration, angiogenesis (Eming, S. A. et al., Frog. Histochem. Cytochem. 42 (2007) 115-170). Skin wounding assays have been used to obtain surrogate tissue to determine PD markers for e.g. anti-angiogenic therapies (Zhang, D. et al., Invest. New Drugs 25 (2006) 49-55; Lockhart, A. C. et al., Clin. Cancer Res. 9 (2003) 586-593). During wound healing macrophages play a substantial role and phenotypic changes of wound associated macrophages (WAM) account for the different roles in the phases of skin repair (e.g. early inflammatory phase=intense phagocytic activity; mid tissue remodelling phase: immunoregulatory state with overexpression of pro-angiogenic factors) (Adamson, R., Journal of Wound Care 18 (2009) 349-351; Rodero, M. P. et al., Int. J. Clin. Exp. Pathol. 25 (2010) 643-653; Brancato, S. K. and Albina, J. E., Wound Macrophages as Key Regulators of Repair, Origin, Phenotype, and Function. AJP (2011), Vol. 178, No. 1).

Indeed, the absence of macrophages resulted in delayed wound healing in genetically engineered mice (Rodero, M. P. et al., Int. J. Clin. Exp. Pathol. 25 (2010) 643-653). Preclinical experiments showed a significant (F4/80 positive) macrophage reduction in the skin of an aCSF-1R treated MDA-MB231 xenograft mouse model. However, species specific differences between mouse and human have been reported (Daley, J. M. et al., J. Leukoc. Biol. 87 (2009) 1-9). As WAMs and TAMs are originating from the same progenitor cells and share similar functions and phenotypes, serial pre-treatment and on-treatment (total of n=4) skin biopsies will be used to analyze the pharmacodynamics effects of humanized Mab 2F11 treatment on WAMs during the wound healing process. Correlation of the skin data with PD effects of humanized Mab 2F11 treatment on TAMs in fresh tumor biopsies can significantly increase knowledge on the molecular basis of how humanized Mab 2F11 works and how the tumor is responding.

In addition, the assessment of wounded skin tissue might potentially substitute for the on-treatment tumor biopsies in later trials and therefore serve as surrogate tissue to assess humanized Mab 2F11 efficacy.

### 3.1.4.3 Rationale for Whole Blood Samples to Measure PD Markers

These surrogate tissue specimens will be used for research purposes to identify biomarkers that are predictive of response to humanized Mab 2F11 treatment (in terms of dose, safety and tolerability) and will help to better understand the pathogenesis, course and outcome of cancer and related diseases. Analysis may include determination of circulating markers associated with the PD activity of humanized Mab 2F11 (e.g. assessment of cytokine levels, circulating immune cells and immune effector cell depletion). Preclinical experiments have shown that changes in e.g. circulating CSF-1, TRAP5b monocyte subpopulations and tissue macrophages are associated with the drug activity. In addition, GLP-Tox data from humanized Mab 2F11 treated cynomolgous monkeys revealed alterations in bone biomarkers of formation (osteocalcin, P1NP), osteoclast activity (TRAP5b) and parathyroid hormone which all correlated with reduced osteoclast numbers. Therefore, these exploratory PD markers and additional circulating immunostimulatory or immunoinhibitory factors will be assessed during the study.

### Tumor Response Criteria

Tumor response will be evaluated according to the RECIST 1.1 criteria In this study, tumor response will be measured using spiral CT scans (including a thoracic scan) or CT scan. X-rays and ultrasound are not acceptable for monitoring target lesions. For each subject, the same method of assessment and the same technique must be used to evaluate each lesion throughout the entire study. If more than one method is used, select the most accurate method according to RECIST when recording data.

Tumor response will be confirmed a minimum of 4 weeks after the initial response was noted, or at the next scheduled tumor assessment if it is to occur more than 4 weeks after the initial response.

An assessment of tumor growth kinetics will be made by comparing post-treatment scans with the last available pre-study scan, if available.

### Pharmacokinetic (PK)/Pharmacodynamic (PD) Assessments

Blood samples will be collected to evaluate the pharmacokinetics PK and/or PD as described in the table below

The total volume blood loss for PK assessments, until the end of Cycle 4, will be approximately 58 mL for Part I, Arm A and Part II and approximately 86 mL for Part I, Arm B. At each subsequent cycle a further 6 mL blood will be collected for PK assessments for each treatment group. The total volume blood loss for PD assessments until the end of Cycle 4 (8 weeks post treatment) will be approximately 161 mL. At each subsequent cycle further 9 ml blood samples (1×5 ml, 1×2 ml and 2×1 ml; see Table 2 for details) will be collected for PD assessments pre-dose.

### PK Assessments

Blood will be collected for analysis of concentrations for humanized Mab 2F11, humanized anti-human antibody (HAHA) to humanized Mab 2F11 and paclitaxel. In addition, a single blood sample will be taken at the time of an infusion-related reaction of significant magnitude and if the infusion is interrupted or the infusion rate is slowed at the discretion of the investigator.

Serum humanized Mab 2F11 and HAHA will be measured using validated assays. All serum samples collected for HAHA determination will also be analyzed for R05509554. All blood samples for PK assessment will be collected from an i.v. line different to that receiving the infusion. Samples intended for humanized Mab 2F11 exposure and HAHA analysis will be split into two separate aliquots, one each for humanized Mab 2F11 and HAHA determination Plasma paclitaxel concentrations will be measured using a validated liquid chromatography tandem mass spectrometry (LC/MS/MS) method.

### PD Assessments

Specimens for dynamic (non inherited) and genetic biomarker (inherited) discovery and validation will be collected from all subjects participating in the trial.

### Whole Blood Samples for PD and Biomarkers

Blood as source tissue will be collected to determine the PD effects of humanized Mab 2F11. All blood samples for PD assessment will be collected from an i.v. line different to that receiving the infusion. PD assessments of whole blood samples will include but are not limited to:
Immunophenotyping (monocyte/macrophage and lymphocyte subsets) using flow cytometry. For monocyte/macrophage subsets these markers include, but are not limited to, CD14, CD16, CD45, MHC class II and for lymphocytes CD3, CD4, CD8, CD16, CD19, CD45, CD56
The total volume blood loss for pharmacodynamic assessments of monocytes/macrophages and lymphocyte cell populations will be approximately 17×5 ml=85 mL for the first four cycles.
Three additional blood samples will be used for the preparation of serum to determine PD related changes of soluble markers. These markers include, but are not limited to:

### Cytokine Assessment A:

CSF-1, Trap5b, sCD163, IL-34

The total volume blood loss for PD Cytokine Assessments A will be approximately 25×2 ml=50 mL for the first four cycles.

### Cytokine Assessment B:

IFNγ, TNFα, IL-1β, IL-4, IL-6, IL-8, IL-10, IL-13, GM-CSF, VEGF, MCP-1, CCL18, CCL22, MIP-1, Galectin 3, IL1Ra, TGF alpha

The total volume blood loss for PD Cytokine Assessments B will be approximately 21×1 ml=21 mL for the first four cycles.

### Bone Biomarkers:

Bone biomarkers such as osteocalcin, P1NP and parathyroid hormone (PTH) will be assessed.

The total volume blood loss will be approximately 5×1 mL=5 mL for the first four cycles.

The largest amount of total volume blood loss per cycle for PD/biomarker assessments will be approximately 51 mL.

### Wound Healing Skin Tissue Biopsies

Surrogate wound healing skin tissue will be analyzed for exploratory PD biomarker analyses associated with wound healing process including but not limited to neutrophil recruitment, macrophage infiltration and angiogenesis (see also 3.1.5). Two skin paired samples will be taken after local anaesthesia from mirror areas of normal skin (preferably located in the back without hair follicles). They will be obtained by using a 2 and a 4 mm diameter punch biopsy device to obtain 2 overlapping samples, which would not require suturing.

The 2 mm biopsy will create the injury and the fully overlapping 4 mm biopsy 7 days later will collect the wound healing material.

The time interval chosen between 2 biopsies is considered to be adequate, based on the understanding of time-course of changes in relevant biomarkers (neutrophil recruitment, macrophage infiltration, angiogenesis) associated with wound healing process (Eming, S. A. et al., Frog. Histochem. Cytochem. 42 (2007) 115-170; Zhang, D. et al., Invest. New Drugs 25 (2006) 49-55; Lockhart, A. C. et al., Clin. Cancer Res. 9 (2003) 586-593).

All skin samples will undergo analysis for:
Hematoxylin & eosin staining (H&E)
Immunohistochemistry (IHC) markers will be analyzed for the following parameters: CSF-1R, CD68/CD163, CD68/MHC class II, CD31 (microvessel density) and Ki67.

The specimens will be formalin fixed and paraffin embedded and shipped to a central laboratory for analysis.

### Tumor Biopsies

### Fresh Tumor Biopsies

Fresh pre-treatment and on-treatment tumor biopsies will be collected to assess pharmacodynamics changes of TAM infiltration and additional tumor markers (see. 3.1.3).

The biopsies should be preferentially taken from the largest metastatic lesion, may be from the primary tumor, or if possible from both primary tumor and a metastatic site and should be biopsied at the tumor-stroma interface if possible.

Collection of tumor biopsies will be guided by ultrasound or CT scan using an 18 gauge needle to provide cores of at least 20 mm in length. At least 2, ideally 4 core biopsies will be obtained at each time point.

One half of the specimen will be formalin fixed and paraffin embedded The second half will be fresh frozen and collected for long term storage for retrospective exploratory analysis of biomarkers (see section 5.5.3.1.2).

Formalin-fixed, paraffin-embedded biopsy samples will be analyzed for:
Hematoxylin and eosin staining (H&E).
Immunohistochemistry (IHC) assessments include, but are not limited to the following markers: CSF-1R, CD68/CD163, CD68/MHC class II, CD31 (microvessel density), Ki67 and other exploratory markers.

### Imaging Modalities for Biomarkers

### DCE-Ultrasound

On the basis of preclinical results we expect that treatment with humanized Mab 2F11 may modulate the microvessel density and the vessel lumen in the tumor and hence the angiogenesis and the transcapillary transport of nutrients to the tumor. To monitor these endpoints, we propose to use DCE-Ultrasound as the choice of imaging modality, where possible.

### FDG-PET

FDG-PET can improve patient management by identifying responders early, before tumor size is reduced; non responders could discontinue futile therapy (Weber, W. A., J. Nucl. Med. 50 (2009) 1S-10S). Moreover, a reduction in the FDG-PET signal within days or weeks of initiating therapy (e.g., in breast (Avril, N. et al., J. Nucl. Med. 50 (2009) 55S-63S), ovarian (Schwarz, J. K. et al., J. Nucl. Med. 50 (2009) 64S-73S), and non-small cell lung (Zander, T. et al., J. Clin. Oncol. (2011) 1701-1708)) significantly correlates with prolonged survival and other clinical end points now used. humanized Mab 2F11 treatment-induced changes in tumor metabolism may be assessed with FUG-PET. In addition, humanized Mab 2F11 induced macrophage depletion may result in the decrease of SUVₘₐₓ in FDG-PET scans.

### Example 15

Inhibition of Tumor Growth Under Treatment with Anti-CSF-1R Monoclonal Antibody in Combination with Chemotherapy or Cancer Immunotherapy in Subcutaneous Syngeneic MC38 Colon Carcinoma Models

Cells of the murine colorectal adenocarcinoma cell line MC-38 (obtained from Beckman Research Institute of the City of Hope, Calif., USA) were cultured in Dulbecco's Modified Eagle Medium (DMEM, PAN Biotech) supplemented with 10% FCS and 2 mM L-glutamine at 37° C. in a water saturated atmosphere at 5% CO2. At the day of inoculation, MC38 tumor cells were harvested with PBS from culture flasks and transferred into culture medium, centrifuged, washed once and re-suspended in PBS. For injection of cells, the final titer was adjusted to 1×107 cells/ml. Subsequently 100 μl of this suspension (1×106 cells) were inoculated subcutaneously into 7-9 weeks old female C57BL/6N mice (obtained from Charles River, Sulzfeld, Germany) Treatment with control antibody (MOPC-21; Bio X Cell, West Lebanon), anti-murine CSF-1R mAb <mouse CSF1R> antibody at a weekly dose of 30 mg/kg i.p. alone or in combination with IL-2 (Proleukin, Novartis, 100 000 IU/animal i.p. twice daily), or FOLFIRI (5-Fluorouracil, Medac, 100 mg/kg, i.p., 1×/Leucovorin, Pfizer, 40 mg/kg, i.p., 1×/Irinotecan, HEXAL, 20 mg/kg, i.p., 1×) or Oxaliplatin (Eloxatin, Sanofi-Aventis 5 mg/kg, i.p. 1×) started after tumors were established and had reached an average size of 50 mm3. Tumor volume was measured twice a week and animal weights were monitored in parallel. In a separate study with comparable set-up, primary tumors from indicated treatment groups were excised, weighed and subjected to FACS analysis. Primary tumor material was collected between study day 20-25 as indicated. To obtain single cell suspensions amenable for flow cytometry analysis the tumors were minced by using the McIlwain tissue chopper. Subsequently, the tumor pieces were resuspended in RPMI media supplemented with collagenase I, dispase II and DNAse I, incubated at 37° C. and cell suspension were passed through a mash. CD45 positive cells were enriched by magnetic cell separation according to the manufacturer's instructions (Miltenyi). Briefly cells were labeled with anti-mouse CD45 conjugated with APC (BD, Cat. No 559864) and separated with anti APC microbeads. To analyse CD8+ T cells these CD45 positive cells were stained with 0.2 μg/ml DAPI (Roche, Cat. No10236276001 and PE conjugated CD8 antibody (eBioscience Cat. No. 12-0081-83) or PE conjugated CD4 antibody (eBioscience, Cat. No. 2-0041-83). Acquisition of data was performed with FACS Canto II and subsequently analysed with FlowJo software. Only viable cells (gated on DAPI-negative cells) were analysed to exclude cell debris and dead cells. Monotherapy with <mouse CSF1R> antibody inhibited primary tumor growth when compared to control antibody treatment (TGI: 61%, TCR: 0.39 CI: 0.15-0.68). Also IL2 monotherapy had an effect on MC38 primary tumor growth (TGI: 47%, TCR: 0.53 CI: 0.27-0.85). Addition of <mouse CSF1R> antibody to IL-2 therapy led to a superior anti-tumor efficacy compared to IL-2 treatment alone (TGI: 78%, TCR: 0.21 CI: 0.02-0.48) Treatment with the chemotherapeutic regimen FOLFIRI also significantly inhibited tumor growth (TGI: 66%, TCR: 0.34 CI: 0.11-0.61) and addition of <mouse CSF1R> antibody led to a further improved outcome (TGI: 77%, TCR: 0.23 CI: 0.001-0.48). Oxaliplatin also showed some but less pronounced efficacy on MC38 tumor growth (TGI: 46%, TCR: 0.54 CI: 0.29-0.86) that nevertheless could be enhanced by combination with the <mouse CSF1R> antibody (TGI: 69%, TCR: 0.31 CI: 0.07-0.59). When looking at the progression of individual tumors above a size of 700 mm3, the median time to progression of animals treated with the combination of <mouse CSF-1R> antibody with IL-2 was superior to combination with chemotherapies in this model (see table 11).

TABLE 11
Anti tumor Efficacy of <mouse CSF1R> antibody combinations
in the MC38 mouse CRC in vivo model

| | | | Median time to |
|---|---|---|---|
| | TGI | TCR | progression |
| Group | (day 21) | (day 21) | TV > 700 mm3 |
| Control (Mouse IgG1) | — | | 17 |
| <mouse CSF1R> | 61% | 0.39 | 21 |
| antibody | | | |
| Oxaliplatin | 46% | 0.54 | 21 |
| FOLFIRI | 66% | 0.34 | 22 |
| Proleukin | 47% | 0.53 | 21 |
| <mouse CSF1R> | 69% | 0.31 | 21 |
| antibody/Eloxatin | | | |
| <mouse CSF1R> | 77% | 0.23 | 27.5 |
| antibody/FOLFIRI | | | |
| <mouse CSF1R> | 78% | 0.22 | 30 |
| antibody/Proleukin | | | |

Flow cytometry analysis of tumors treated with <mouse CSF-1R antibody> revealed a 3-fold increase in the numbers of CD8+ T cells compared to Oxaliplatin monotherapy as well as a slight increase in CD4+ T cells. Tumors treated with the combination of CSF-1R neutralizing antibody and Oxaliplatin showed a comparable increase of T cells when treated with antibody alone. Similar results were obtained for the combination with FOLFIRI. Results are also shown in FIG. 5.

### Example 16

Inhibition of Tumor Growth Under Treatment with Anti-CSF-1R Monoclonal Antibody in Combination with Anti-CD40 Monoclonal Antibody in Subcutaneous Syngeneic MC38 Colon Carcinoma Model

Cells of the murine colorectal adenocarcinoma cell line MC-38 (obtained from Beckman Research Institute of the City of Hope, Calif., USA) were cultured in Dulbecco's Modified Eagle Medium (DMEM, PAN Biotech) supplemented with 10% FCS and 2 mM L-glutamine at 37° C. in a water saturated atmosphere at 5% CO2. At the day of inoculation, MC38 tumor cells were harvested with PBS from culture flasks and transferred into culture medium, centrifuged, washed once and re-suspended in PBS. For injection of cells, the final titer is adjusted to 1×107 cells/ml. Subsequently 100 μl of this suspension (1×106 cells) were inoculated subcutaneously into 6-10 weeks old female C57BL/6N mice. Groups of animals were treated with control antibodies (MOPC-21 (30 mg/kg i.p. once weekly) and 2A3 (100 μg i.p. once); Bio X Cell, West Lebanon), anti-murine CSF-1R mAb <mouse CSF1R> antibody (30 mg/kg i.p. once weekly) alone or in combination with anti-CD40 monoclonal antibody FGK45 (agonist CD40 rat anti-mouse IgG2a mAb FGK45 (S. P. Schoenberger, et al, Nature, 393, 480 (1998), available from BioXcell) CD40 (FGK45)) (100 μg, i.p., 1×). Treatment started after tumors were established and had reached an average size of 50 mm3 Tumor volume was measured twice a week and animal weights were monitored in parallel. Results are shown in FIG. 7. Combination of CSF1R mAb+CD40 mAb FGK45 shows improved anti-tumor efficacy over monotherapies in syngeneic MC38 mouse colon cancer model

### Example 17

Inhibition of Tumor Growth Under Treatment with Anti-CSF-1R Monoclonal Antibody in Combination with Anti-Ang2/VEGF Monoclonal Antibody and/or FOLFIRI in Subcutaneous Syngeneic MC38 Colon Carcinoma Model

Cells of the murine colorectal adenocarcinoma cell line MC-38 (obtained from Beckman Research Institute of the City of Hope, Calif., USA) are cultured in Dulbecco's Modified Eagle Medium (DMEM, PAN Biotech) supplemented with 10% FCS and 2 mM L-glutamine at 37° C. in a water saturated atmosphere at 5% CO2. At the day of inoculation, MC38 tumor cells are harvested with PBS from culture flasks and transferred into culture medium, centrifuged, washed once and re-suspended in PBS. For injection of cells, the final titer is adjusted to 1×107 cells/ml. Subsequently 100 μl of this suspension (1×106 cells) are inoculated subcutaneously into 7 weeks old female C57BL/6N mice. Treatment with control antibody (MOPC-21; Bio X Cell, West Lebanon), anti-murine CSF-1R mAb <mouse CSF1R> antibody at a weekly dose of 30 mg/kg i.p. alone or in combination with FOLFIRI (5-Fluorouracil, Medac, 100 mg/kg, i.p., 1×/Leucovorin, Pfizer, 40 mg/kg, i.p., 1×/Irinotecan, HEXAL, 20 mg/kg, i.p., 1×) or anti-Ang2/VEGF monoclonal antibody (the bispecific ANG-2-VEGF antibody XMab1 as described in WO2011/117329) (10 mg/kg, i.p., 1× weekly) starts after tumors are established and have reached an average size of 50 mm³. Triple combination treatment is performed as described in Table 13. Tumor volume is measured twice a week and animal weights are monitored in parallel. Monotherapy with <mouse CSF1R> antibody, anti-Ang2/VEGF antibody or FOLFIRI minimally inhibited primary tumor growth when compared to control antibody treatment (TGI: 28%, 35% or 11%, respectively). Combination of <mouse CSF1R> antibody with either anti-Ang2/VEGF antibody or FOLFIRI led to more pronounced and statistically significant anti-tumor efficacy compared to the control antibody (TGI: 64% or 67%) Triple combination treatment of <mouse CSF-1R> antibody with FOLFIRI followed by the treatment with the anti-Ang2/VEGF antibody 2 days or 9 days thereafter showed the best anti-tumor activity (TGI: 68% or 70%). Concurrent treatment of the 3 compounds or combination of the anti-Ang2/VEGF antibody with FOLFIRI followed by the treatment with of <mouse CSF-1R> antibody 9 days thereafter just yielded an anti-tumor activity of 61% or 56%, respectively. When looking at the progression of individual tumors above a size of 700 mm3, the median time to progression of animals treated with the combination of <mouse CSF-1R> antibody with FOLFIRI followed by the treatment with the anti-Ang2/VEGF antibody 2 days or 9 days thereafter was also superior to the median time to progression of all other treatments in this model (see table 12).

TABLE 13
Anti tumor Efficacy of <mouse CSF1R> antibody in
combination with anti-Ang2/VEGF monoclonal antibody
and/or FOLFIRI in the MC38 mouse CRC in vivo model

| | | | Median time to |
|---|---|---|---|
| | TGI | TCR | progression |
| Group | (day 20) | (day 20) | TV > 700 mm3 |
| Control (Mouse IgG1) | — | — | 20 |
| <mouse CSF1R> | 28% | 0.72 | 22 |
| antibody | | | |
| anti-Ang2/VEGF | 35% | 0.65 | 23 |
| antibody | | | |
| FOLFIRI | 11% | 0.84 | 20 |
| <mouse CSF1R> | 67% | 0.34 | 26 |
| antibody/FOLFIRI | | | |
| anti-Ang2/VEGF | 43% | 0.52 | 24 |
| antibody/FOLFIRI | | | |
| <mouse CSF1R> | 64% | 0.35 | 27 |
| antibody/anti- | | | |
| Ang2/VEGF antibody | | | |
| <mouse CSF1R> | 61% | 0.39 | 26 |
| antibody/FOLFIRI/anti- | | | |
| Ang2/VEGF antibody; | | | |
| concurrent treatment | | | |
| <mouse CSF1R> | 68 | 0.27 | 28 |
| antibody (day | | | |
| 7)/FOLFIRI/anti- | | | |
| Ang2/VEGF antibody | | | |
| (day 9) | | | |
| <mouse CSF1R> | 70% | 0.22 | 28 |
| antibody (day | | | |
| 7)//FOLFIRI/anti- | | | |
| Ang2/VEGF antibody | | | |
| (day 16) | | | |
| <mouse CSF1R> | 56 | 0.43 | 26 |
| antibody (day | | | |
| 16)/FOLFIRI/anti- | | | |
| Ang2/VEGF antibody | | | |
| (day 7) | | | |

1. A method of inhibiting
a) proliferation of CSF-1R ligand-dependent and/or CSF-1R ligand-independent CSF-1R expressing tumor cells;
b) proliferation of tumors with CSF-1 ligand-dependent and/or CSF-1 ligand-independent CSF-1R expressing macrophage infiltrate;
c) cell survival (in CSF-1R ligand-dependent and/or CSF-1R ligand-independent) CSF-1R expressing monocytes and macrophages;
d) cell differentiation (in CSF-1R ligand-dependent and/or CSF-1R ligand-independent) CSF-1R expressing monocytes into macrophages; or
e) a combination thereof
the method comprising administering to a patient an anti-CSF-1R antibody that specifically binds to the (dimerization) domains D4 to D5 (SEQ ID No: 85) of the extracellular domain of human CSF-1R in combination with a chemotherapeutic agent, radiation, cancerimmunotherapy, and combinations thereof

2. A method of treating a patient having a CSF-1R expressing tumor or having a tumor with CSF-1R expressing macrophage infiltrate, wherein the tumor is characterized by an increase of CSF-1R ligand the method comprising
administering a therapy comprising an effective amount of an anti-CSF-1R antibody that specifically binds to the domains D4 to D5 (SEQ ID No: 85) of the extracellular domain of human CSF-1R,
and a chemotherapeutic agent, radiation, cancer immunotherapy, and combinations thereof.

3. The method according to claim 1, wherein the chemotherapeutic agent is selected from taxanes (paclitaxel (Taxol), docetaxel (Taxotere), modified paclitaxel (Abraxane and Opaxio)), doxorubicin, modified doxorubicin (Caelyx or Doxil)), sunitinib (Sutent), sorafenib (Nexavar), and other multikinase inhibitors, oxaliplatin, cisplatin, carboplatin, etoposide, gemcitabine, and vinblastine.

4. The method according to claim 1, wherein the cancer immunotherapy is selected from:
a) T cell engaging agents selected from agonistic antibodies which bind to human OX40, TO GITR, TO CD27, OR TO 4-1BB, and T-cell bispecific antibodies (e.g. T cell-engaging BiTE™ antibodies CD3-CD19, CD3-EpCam, CD3-EGFR), IL-2 (Proleukin), Interferon (IFN) alpha, antagonizing antibodies which bind to human CTLA-4, to PD-1, to PD-L1, to TIM-3, to BTLA, to VISTA, to LAG-3, or to CD25,
b) targeting immunosuppression: antibodies or small molecules targeting STAT3 or NFkB signaling, blocking IL-6, IL-17, IL-23, TNFa function,
c) cancer vaccines/enhance dendritic cell function: oncolytic virus secreting GM-CSF (OncoVex), an agonistic CD40 antibody, Toll-like receptor (TLR) ligands, TLR agonists, recombinant fusion protein encoding MAGE-A3, PROSTVAC; or
d) adoptive cell transfer: GVAX (prostate cancer cell line expressing GM-CSF), dendritic cell vaccine, adoptive T cell therapy, adoptive CAR T cell therapy.

5. The method according to claim 4, wherein the cancer immunotherapy is an agonistic CD40 antibody.

6. The method according to claim 1, wherein the chemotherapeutic agent is selected from taxanes (docetaxel or paclitaxel or a modified paclitaxel (Abraxane or Opaxio)), doxorubicin, capecitabine. bevacizumab, and combinations thereof and the patient has been diagnosed with breast cancer.

7. The method according to claim 1, wherein the chemotherapeutic agent is selected from carboplatin, oxaliplatin, cisplatin, paclitaxel, doxorubicin (or modified doxorubicin (Caelyx or Doxil)), topotecan (Hycamtin), and combinations thereof and further wherein the patient has been diagnosed with ovarian cancer.

8. The method according to claim 1, wherein the chemotherapeutic agent is selected from multi-kinase inhibitor (sunitinib (Sutent), sorafenib (Nexavar) or motesanib diphosphate (AMG 706), doxorubicin, and combinations thereof and further wherein the patient has been diagnosed with renal cancer.

9. The method according to claim 1, wherein the chemotherapeutic agent is selected from oxaliplatin, cisplatin, radiation, and combinations thereof and the patient has been diagnosed with squamous cell carcinoma.

10. The method according to claim 1, wherein the chemotherapeutic agent is selected from taxol, carboplatin, and combinations thereof and the patient has been diagnosed with lung cancer.

11. The method according to claim 1, wherein the antibody does not bind to human CSF-1R fragment delD4 (SEQ ID NO: 65).

12. The method according to claim 1, wherein the antibody binds to human CSF-1R fragment delD4 (SEQ ID NO: 65) and to human CSF-1R Extracellular Domain (SEQ ID NO: 64) with a ratio of 1:50 or lower.

13. The method according to claim 1, wherein the antibody comprises
a) a heavy chain variable domain comprising SEQ ID NO:7 and the light chain variable domain comprising SEQ ID NO:8,
b) a heavy chain variable domain comprising SEQ ID NO:15 and the light chain variable domain comprising SEQ ID NO:16;
c) a heavy chain variable domain comprising SEQ ID NO:75 and the light chain variable domain comprising SEQ ID NO:76;
d) a heavy chain variable domain comprising SEQ ID NO:83 and the light chain variable domain comprising SEQ ID NO:84;
or a humanized version thereof.

14. The method according to claim 1, wherein the antibody comprises
a) a heavy chain variable domain comprising SEQ ID NO:23 and the light chain variable domain comprising SEQ ID NO:24, or
b) a heavy chain variable domain comprising SEQ ID NO:31 and the light chain variable domain comprising SEQ ID NO:32, or
c) a heavy chain variable domain comprising SEQ ID NO:39 and the light chain variable domain comprising SEQ ID NO:40, or
d) a heavy chain variable domain comprising SEQ ID NO:47 and the light chain variable domain comprising SEQ ID NO:48, or
e) a heavy chain variable domain comprising SEQ ID NO:55 and the light chain variable domain comprising SEQ ID NO:56.

15. The method according to claim 1, wherein the antibody comprises
a) a heavy chain variable domain comprising a CDR3 region of SEQ ID NO: 1, a CDR2 region of SEQ ID NO: 2, and a CDR1 region of SEQ ID NO:3, and a light chain variable domain comprising a CDR3 region of SEQ ID NO: 4, a CDR2 region of SEQ ID NO:5, and a CDR1 region of SEQ ID NO:6, or
b) a heavy chain variable domain comprising a CDR3 region of SEQ ID NO: 9, a CDR2 region of SEQ ID NO: 10, and a CDR1 region of SEQ ID NO: 11, and a light chain variable domain comprising a CDR3 region of SEQ ID NO:12, a CDR2 region of SEQ ID NO: 13, and a CDR1 region of SEQ ID NO: 14, or
c) a heavy chain variable domain comprising a CDR3 region of SEQ ID NO: 17, a CDR2 region of SEQ ID NO: 18, and a CDR1 region of SEQ ID NO:19, and a light chain variable domain comprising a CDR3 region of SEQ ID NO: 20, a CDR2 region of SEQ ID NO:21, and a CDR1 region of SEQ ID NO:22, or
d) a heavy chain variable domain comprising a CDR3 region of SEQ ID NO: 25, a CDR2 region of SEQ ID NO: 26, and a CDR1 region of SEQ ID NO: 27, and a light chain variable domain comprising a CDR3 region of SEQ ID NO:28, a CDR2 region of SEQ ID NO: 29, and a CDR1 region of SEQ ID NO: 30, or
e) a heavy chain variable domain comprising a CDR3 region of SEQ ID NO: 33, a CDR2 region of SEQ ID NO: 34, and a CDR1 region of SEQ ID NO: 35, and a light chain variable domain comprising a CDR3 region of SEQ ID NO:36, a CDR2 region of SEQ ID NO: 37, and a CDR1 region of SEQ ID NO: 38, or
f) a heavy chain variable domain comprising a CDR3 region of SEQ ID NO:41, a CDR2 region of SEQ ID NO: 42, and a CDR1 region of SEQ ID NO:43, and a light chain variable domain comprising a CDR3 region of SEQ ID NO: 44, a CDR2 region of SEQ ID NO:45, and a CDR1 region of SEQ ID NO:46, or
g) a heavy chain variable domain comprising a CDR3 region of SEQ ID NO: 49, a CDR2 region of SEQ ID NO: 50, and a CDR1 region of SEQ ID NO: 51, and a light chain variable domain comprising a CDR3 region of SEQ ID NO:52, a CDR2 region of SEQ ID NO: 53, and a CDR1 region of SEQ ID NO: 54; or
h) a heavy chain variable domain comprising a CDR3 region of SEQ ID NO:69, a CDR2 region of SEQ ID NO: 70, and a CDR1 region of SEQ ID NO:71, and a light chain variable domain comprising a CDR3 region of SEQ ID NO: 72, a CDR2 region of SEQ ID NO:73, and a CDR1 region of SEQ ID NO:74, or
i) a heavy chain variable domain comprising a CDR3 region of SEQ ID NO: 77, a CDR2 region of SEQ ID NO: 78, and a CDR1 region of SEQ ID NO: 79, and a light chain variable domain comprising a CDR3 region of SEQ ID NO:80, a CDR2 region of SEQ ID NO: 81, and a CDR1 region of SEQ ID NO: 82.

16. The method to claim 1, wherein said antibody is a human IgG1 or a human IgG4.

17. A method for treating a patient having a CSF-1R expressing tumor or having a tumor with CSF-1R expressing macrophage infiltrate, wherein the tumor is characterized by an increase of CSF-1R ligand, the method comprising administering an antibody that specifically binds to human CSF-1R and a cancer immunotherapy, wherein the cancer immunotherapy is selected from the group consisting of:
a) T cell engaging agents selected from the group consisting of agonistic antibodies which bind to human OX40, to GITR, to CD27, or to 4-1BB, and T-cell bispecific antibodies (e.g. T cell-engaging BiTE™ antibodies CD3-CD19, CD3-EpCam, CD3-EGFR), IL-2 (Proleukin), Interferon (IFN) alpha, antagonizing antibodies which bind to human CTLA-4 (e.g. ipilimumab), to PD-1, to PD-L1, to TIM-3, to BTLA, to VISTA, to LAG-3, or to CD25;
b) targeting immunosuppression: antibodies or small molecules targeting STAT3 or NFkB signaling, blocking IL-6, IL-17, IL-23, TNFa function;
c) cancer vaccines/enhance dendritic cell function: OncoVex (oncolytic virus secreting GM-CSF), an agonistic CD40 antibody, Toll-like receptor (TLR) ligands, TLR agonists, recombinant fusion protein encoding MAGE-A3, PROSTVAC; and
d) adoptive cell transfer: GVAX (prostate cancer cell line expressing GM-CSF), dendritic cell vaccine, adoptive T cell therapy, adoptive CAR T cell therapy.

18. The method according to claim 17 wherein the cancer immunotherapy is selected from the group consisting of:
cancer vaccines/enhance dendritic cell function: OncoVex (oncolytic virus secreting GM-CSF), an agonistic CD40 antibody, Toll-like receptor (TLR) ligands, TLR agonists, recombinant fusion protein encoding MAGE-A3, and PROSTVAC.

19. The method according to claim 17, wherein the cancer immunotherapy is an agonistic CD40 antibody.

20. A method for determining whether a subject having a cancer is a candidate for an anti-CSF-1R antibody-based cancer treatment regimen, the method comprising:
ex vivo or in vitro determining in vitro the level of one or more of the following markers: CSF-1R, CD68/CD163, CD68/MHC class II, CD31 (microvessel density), and Ki67 and other markers like e.g. immuninfiltrates;
in a sample of the subject, wherein the sample is selected from the group consisting of tissue, blood, serum, plasma, tumor cells and circulating tumor cells; and
wherein a change in the level of one or more of CSF-1R, CD68/CD163, CD68/MHC class II, CD31 (microvessel density) and Ki67 and other markers like e.g. immuninfiltrates (e.g. T cells (e.g. CD4- and/or CD8-T cells), as compared with to the corresponding level in an individual not suffering from cancer, is indicative that the subject is a candidate for the anti-CSF-1 R antibody-based cancer treatment regimen.

21. The method of claim 20, wherein the antibody used in said regimen is an antibody comprising
a) a heavy chain variable domain comprising a CDR3 region of SEQ ID NO: 1, a CDR2 region of SEQ ID NO: 2, and a CDR1 region of SEQ ID NO:3, and a light chain variable domain comprising a CDR3 region of SEQ ID NO: 4, a CDR2 region of SEQ ID NO:5, and a CDR1 region of SEQ ID NO:6, or
b) a heavy chain variable domain comprising a CDR3 region of SEQ ID NO: 9, a CDR2 region of SEQ ID NO: 10, and a CDR1 region of SEQ ID NO: 11, and a light chain variable domain comprising a CDR3 region of SEQ ID NO: 12, a CDR2 region of SEQ ID NO: 13, and a CDR1 region of SEQ ID NO: 14, or
c) a heavy chain variable domain comprising a CDR3 region of SEQ ID NO: 17, a CDR2 region of SEQ ID NO: 18, and a CDR1 region of SEQ ID NO:19, and a light chain variable domain comprising a CDR3 region of SEQ ID NO: 20, a CDR2 region of SEQ ID NO:21, and a CDR1 region of SEQ ID NO:22, or
d) a heavy chain variable domain comprising a CDR3 region of SEQ ID NO: 25, a CDR2 region of SEQ ID NO: 26, and a CDR1 region of SEQ ID NO: 27, and a light chain variable domain comprising a CDR3 region of SEQ ID NO:28, a CDR2 region of SEQ ID NO: 29, and a CDR1 region of SEQ ID NO: 30, or
e) a heavy chain variable domain comprising a CDR3 region of SEQ ID NO: 33, a CDR2 region of SEQ ID NO: 34, and a CDR1 region of SEQ ID NO: 35, and a light chain variable domain comprising a CDR3 region of SEQ ID NO:36, a CDR2 region of SEQ ID NO: 37, and a CDR1 region of SEQ ID NO: 38, or
f) a heavy chain variable domain comprising a CDR3 region of SEQ ID NO:41, a CDR2 region of SEQ ID NO: 42, and a CDR1 region of SEQ ID NO:43, and a light chain variable domain comprising a CDR3 region of SEQ ID NO: 44, a CDR2 region of SEQ ID NO:45, and a CDR1 region of SEQ ID NO:46, or
g) a heavy chain variable domain comprising a CDR3 region of SEQ ID NO: 49, a CDR2 region of SEQ ID NO: 50, and a CDR1 region of SEQ ID NO: 51, and a light chain variable domain comprising a CDR3 region of SEQ ID NO:52, a CDR2 region of SEQ ID NO: 53, and a CDR1 region of SEQ ID NO: 54; or
h) a heavy chain variable domain comprising a CDR3 region of SEQ ID NO:69, a CDR2 region of SEQ ID NO: 70, and a CDR1 region of SEQ ID NO:71, and a light chain variable domain comprising a CDR3 region of SEQ ID NO: 72, a CDR2 region of SEQ ID NO:73, and a CDR1 region of SEQ ID NO:74, or
i) a heavy chain variable domain comprising a CDR3 region of SEQ ID NO: 77, a CDR2 region of SEQ ID NO: 78, and a CDR1 region of SEQ ID NO: 79, and a light chain variable domain comprising a CDR3 region of SEQ ID NO:80, a CDR2 region of SEQ ID NO: 81, and a CDR1 region of SEQ ID NO: 82.

22. The method of claim 20, wherein in this method the change in the level of CSF-1R, CD68/CD163, CD68/MHC class II, CD31 (microvessel density) and Ki67 and other markers like e.g. immuninfiltrates (e.g. T cells (e.g. CD4- and/or CD8-T cells), as compared to the level in an individual not suffering from cancer is an increase in the level of one or more of these markers.

23. A method for determining whether a subject having a cancer is a candidate for a therapy comprising an anti-CSF-1R antibody, the method comprising:
ex vivo or in vitro determining in vitro the level of one or more of the following markers:
CSF-1, Trap5b, sCD163, IL-34;
in a sample of the subject, wherein the sample is selected from the group consisting of tissue, blood, serum, plasma, tumor cells and circulating tumor cells; and
wherein a change in the level of one or more of CSF-1, Trap5b, sCD163, IL-34, as compared with to the corresponding level in an individual not suffering from cancer, is indicative that the subject is a candidate for the therapy.

24. The method of claim 23, wherein the antibody used in said regimen is an antibody comprising
a) a heavy chain variable domain comprising a CDR3 region of SEQ ID NO: 1, a CDR2 region of SEQ ID NO: 2, and a CDR1 region of SEQ ID NO:3, and a light chain variable domain comprising a CDR3 region of SEQ ID NO: 4, a CDR2 region of SEQ ID NO:5, and a CDR1 region of SEQ ID NO:6, or
b) a heavy chain variable domain comprising a CDR3 region of SEQ ID NO: 9, a CDR2 region of SEQ ID NO: 10, and a CDR1 region of SEQ ID NO: 11, and a light chain variable domain comprising a CDR3 region of SEQ ID NO: 12, a CDR2 region of SEQ ID NO: 13, and a CDR1 region of SEQ ID NO: 14, or
c) a heavy chain variable domain comprising a CDR3 region of SEQ ID NO: 17, a CDR2 region of SEQ ID NO: 18, and a CDR1 region of SEQ ID NO:19, and a light chain variable domain comprising a CDR3 region of SEQ ID NO: 20, a CDR2 region of SEQ ID NO:21, and a CDR1 region of SEQ ID NO:22, or
d) a heavy chain variable domain comprising a CDR3 region of SEQ ID NO: 25, a CDR2 region of SEQ ID NO: 26, and a CDR1 region of SEQ ID NO: 27, and a light chain variable domain comprising a CDR3 region of SEQ ID NO:28, a CDR2 region of SEQ ID NO: 29, and a CDR1 region of SEQ ID NO: 30, or
e) a heavy chain variable domain comprising a CDR3 region of SEQ ID NO: 33, a CDR2 region of SEQ ID NO: 34, and a CDR1 region of SEQ ID NO: 35, and a light chain variable domain comprising a CDR3 region of SEQ ID NO:36, a CDR2 region of SEQ ID NO: 37, and a CDR1 region of SEQ ID NO: 38, or
f) a heavy chain variable domain comprising a CDR3 region of SEQ ID NO:41, a CDR2 region of SEQ ID NO: 42, and a CDR1 region of SEQ ID NO:43, and a light chain variable domain comprising a CDR3 region of SEQ ID NO: 44, a CDR2 region of SEQ ID NO:45, and a CDR1 region of SEQ ID NO:46, or
g) a heavy chain variable domain comprising a CDR3 region of SEQ ID NO: 49, a CDR2 region of SEQ ID NO: 50, and a CDR1 region of SEQ ID NO: 51, and a light chain variable domain comprising a CDR3 region of SEQ ID NO:52, a CDR2 region of SEQ ID NO: 53, and a CDR1 region of SEQ ID NO: 54; or
h) a heavy chain variable domain comprising a CDR3 region of SEQ ID NO:69, a CDR2 region of SEQ ID NO: 70, and a CDR1 region of SEQ ID NO:71, and a light chain variable domain comprising a CDR3 region of SEQ ID NO: 72, a CDR2 region of SEQ ID NO:73, and a CDR1 region of SEQ ID NO:74, or
i) a heavy chain variable domain comprising a CDR3 region of SEQ ID NO: 77, a CDR2 region of SEQ ID NO: 78, and a CDR1 region of SEQ ID NO: 79, and a light chain variable domain comprising a CDR3 region of SEQ ID NO:80, a CDR2 region of SEQ ID NO: 81, and a CDR1 region of SEQ ID NO: 82.

25. The method of claim 23, wherein in this method the change in the level of CSF-1, Trap5b, sCD163, IL-34, as compared to the level in an individual not suffering from cancer is an increase in the level of one or more of these markers.

26. The method of claim 23, wherein in this method ex vivo or in vitro the level and change of the level of sCD163 is determined.

27. A method for determining whether a subject having a cancer is a candidate for an anti-CSF-1R antibody-based cancer treatment regimen, the method comprising:
ex vivo or in vitro determining in vitro the level of one or more of the following markers:
IFNγ, TNFα, IL-1β, IL-4, IL-6, IL-8, IL-10, IL-13, GM-CSF, VEGF, MCP-1, CCL18, CCL22, MIP-1, Galectin 3, IL1Ra, TGF alpha;
in a sample of the subject, wherein the sample is selected from the group consisting of tissue, blood, serum, plasma, tumor cells and circulating tumor cells; and
wherein a change in the level of one or more of IFNγ, TNFα, IL-1β, IL-4, IL-6, IL-8, IL-10, IL-13, GM-CSF, VEGF, MCP-1, CCL18, CCL22, MIP-1, Galectin 3, IL1Ra, TGF alpha, as compared with to the corresponding level in an individual not suffering from cancer, is indicative that the subject is a candidate for the anti-CSF-1 R antibody-based cancer treatment regimen.

28. The method of claim 27, wherein the antibody used in said regimen is an is an antibody comprising
a) a heavy chain variable domain comprising a CDR3 region of SEQ ID NO: 1, a CDR2 region of SEQ ID NO: 2, and a CDR1 region of SEQ ID NO:3, and a light chain variable domain comprising a CDR3 region of SEQ ID NO: 4, a CDR2 region of SEQ ID NO:5, and a CDR1 region of SEQ ID NO:6, or
b) a heavy chain variable domain comprising a CDR3 region of SEQ ID NO: 9, a CDR2 region of SEQ ID NO: 10, and a CDR1 region of SEQ ID NO: 11, and a light chain variable domain comprising a CDR3 region of SEQ ID NO: 12, a CDR2 region of SEQ ID NO: 13, and a CDR1 region of SEQ ID NO: 14, or
c) a heavy chain variable domain comprising a CDR3 region of SEQ ID NO: 17, a CDR2 region of SEQ ID NO: 18, and a CDR1 region of SEQ ID NO:19, and a light chain variable domain comprising a CDR3 region of SEQ ID NO: 20, a CDR2 region of SEQ ID NO:21, and a CDR1 region of SEQ ID NO:22, or
d) a heavy chain variable domain comprising a CDR3 region of SEQ ID NO: 25, a CDR2 region of SEQ ID NO: 26, and a CDR1 region of SEQ ID NO: 27, and a light chain variable domain comprising a CDR3 region of SEQ ID NO:28, a CDR2 region of SEQ ID NO: 29, and a CDR1 region of SEQ ID NO: 30, or
e) a heavy chain variable domain comprising a CDR3 region of SEQ ID NO: 33, a CDR2 region of SEQ ID NO: 34, and a CDR1 region of SEQ ID NO: 35, and a light chain variable domain comprising a CDR3 region of SEQ ID NO:36, a CDR2 region of SEQ ID NO: 37, and a CDR1 region of SEQ ID NO: 38, or
f) a heavy chain variable domain comprising a CDR3 region of SEQ ID NO:41, a CDR2 region of SEQ ID NO: 42, and a CDR1 region of SEQ ID NO:43, and a light chain variable domain comprising a CDR3 region of SEQ ID NO: 44, a CDR2 region of SEQ ID NO:45, and a CDR1 region of SEQ ID NO:46, or
g) a heavy chain variable domain comprising a CDR3 region of SEQ ID NO: 49, a CDR2 region of SEQ ID NO: 50, and a CDR1 region of SEQ ID NO: 51, and a light chain variable domain comprising a CDR3 region of SEQ ID NO:52, a CDR2 region of SEQ ID NO: 53, and a CDR1 region of SEQ ID NO: 54; or
h) a heavy chain variable domain comprising a CDR3 region of SEQ ID NO:69, a CDR2 region of SEQ ID NO: 70, and a CDR1 region of SEQ ID NO:71, and a light chain variable domain comprising a CDR3 region of SEQ ID NO: 72, a CDR2 region of SEQ ID NO:73, and a CDR1 region of SEQ ID NO:74, or
i) a heavy chain variable domain comprising a CDR3 region of SEQ ID NO: 77, a CDR2 region of SEQ ID NO: 78, and a CDR1 region of SEQ ID NO: 79, and a light chain variable domain comprising a CDR3 region of SEQ ID NO:80, a CDR2 region of SEQ ID NO: 81, and a CDR1 region of SEQ ID NO: 82.

29. The method of claim 27 wherein in this method the change in the level of IFNγ, TNFα, IL-1β, IL-4, IL-6, IL-8, IL-10, IL-13, GM-CSF, VEGF, MCP-1, CCL18, CCL22, MIP-1, Galectin 3, IL1Ra, TGF alpha, as compared to the level in an individual not suffering from cancer is an increase in the level of one or more of these markers.

30. A method of treating cancer, the method comprising administering therapy comprising an anti-CSF-1R antibody and a bispecific ANG-2-VEGF antibody.

31. A method of treating cancer, the method comprising administering therapy comprising an anti-CSF-1R antibody and an agonistic CD40 antibody.

32. The method according to claim 31,
i) wherein the anti-CSF-1R antibody comprises (a) a heavy chain variable domain amino acid sequence of SEQ ID NO:39 and (b) a light chain variable domain amino acid sequence of SEQ ID NO:40; and
ii) wherein the agonistic CD40 antibody comprises (a) a heavy chain variable domain amino acid sequence of SEQ ID NO: 88 and (b) a light chain variable domain amino acid sequence of SEQ ID NO: 89.

33. The method according to claim 31, wherein the anti-CSF-1R antibody comprises (a) a heavy chain variable domain amino acid sequence of SEQ ID NO:39 and (b) a light chain variable domain amino acid sequence of SEQ ID NO:40; and wherein the agonistic CD40 antibody is dacetuzumab.

34. The method according to claim 31,
i) wherein the anti-CSF-1R antibody comprises (a) a heavy chain variable domain amino acid sequence of SEQ ID NO:39 and (b) a light chain variable domain amino acid sequence of SEQ ID NO:40; and
ii) wherein the agonistic CD40 antibody comprises (a) a heavy chain variable domain amino acid sequence of SEQ ID NO: 90 and (b) a light chain variable domain amino acid sequence of SEQ ID NO: 91.
